SEQUENCE LISTING (123 sequences)

SEQ ID NO: 1 - DNA, 25 bp, synthetic construct
  misc_feature 1..25: note = Artificial
  source 1..25: mol_type = other DNA
tttaccgggaattgtcgaattaggc

SEQ ID NO: 2 - DNA, 16 bp, synthetic construct
  misc_feature 1..16: note = Artificial
  source 1..16: mol_type = other DNA
gtgatctgaccgccgg

SEQ ID NO: 3 - DNA, 24 bp, synthetic construct
  misc_feature 1..24: note = Artificial
  source 1..24: mol_type = other DNA
tttgccttggattttccacttgca

SEQ ID NO: 4 - DNA, 23 bp, synthetic construct
  misc_feature 1..23: note = Artificial
  source 1..23: mol_type = other DNA
gagttggattcgtggccgcgcgg

SEQ ID NO: 5 - DNA, 23 bp, synthetic construct
  misc_feature 1..23: note = Artificial
  source 1..23: mol_type = other DNA
tggtgggagatccatctgcgagg

SEQ ID NO: 6 - DNA, 25 bp, synthetic construct
  misc_feature 1..25: note = Artificial
  source 1..25: mol_type = other DNA
tttctcatactcgttggctcgttgc

SEQ ID NO: 7 - DNA, 20 bp, synthetic construct
  misc_feature 1..20: note = Artificial
  source 1..20: mol_type = other DNA
cccatcctccagtgctacgg

SEQ ID NO: 8 - DNA, 28 bp, synthetic construct
  misc_feature 1..28: note = Artificial
  source 1..28: mol_type = other DNA
tttaggacctctagaggctacttctgcc

SEQ ID NO: 9 - DNA, 27 bp, synthetic construct
  misc_feature 1..27: note = Artificial
  source 1..27: mol_type = other DNA
tttgataatagctatgaagattttgtg

SEQ ID NO: 10 - DNA, 30 bp, synthetic construct
  misc_feature 1..30: note = Artificial
  source 1..30: mol_type = other DNA
gccgaccctttggccacgtggcaatatata

SEQ ID NO: 11 - DNA, 30 bp, synthetic construct
  misc_feature 1..30: note = Artificial
  source 1..30: mol_type = other DNA
gccgacgccacgtggcaagccgcctatata

SEQ ID NO: 12 - DNA, 20 bp, synthetic construct
  misc_feature 1..20: note = Artificial
  source 1..20: mol_type = other DNA
tgtctcgccgccgcgggaaa

SEQ ID NO: 13 - DNA, 36 bp, synthetic construct
  misc_feature 1..36: note = Artificial
  source 1..36: mol_type = other DNA
tgtctcgccgccgcgggaaaagccgacgcgggaaaa

SEQ ID NO: 14 - DNA, 36 bp, synthetic construct; identical to SEQ ID NO: 13
  misc_feature 1..36: note = Artificial
  source 1..36: mol_type = other DNA

SEQ ID NO: 15 - DNA, 39 bp, synthetic construct
  misc_feature 1..39: note = Artificial
  source 1..39: mol_type = other DNA
gctcaagccgacgcgggaaaggccgacgccgccgccgac

SEQ ID NO: 16 - DNA, 30 bp, synthetic construct
  misc_feature 1..30: note = Artificial
  source 1..30: mol_type = other DNA
cctttgtatatagccgacgccgacgccgcc

SEQ ID NO: 17 - DNA, 24 bp, synthetic construct
  misc_feature 1..24: note = Artificial
  source 1..24: mol_type = other DNA
cctttgtatatagccgacgccgac

SEQ ID NO: 18 - DNA, 30 bp, synthetic construct; identical to SEQ ID NO: 16
  misc_feature 1..30: note = Artificial
  source 1..30: mol_type = other DNA

SEQ ID NO: 19 - DNA, 36 bp, synthetic construct
  misc_feature 1..36: note = Artificial
  source 1..36: mol_type = other DNA
gcatgccgaccctttggccacgtggcaatatatatt

SEQ ID NO: 20 - DNA, 36 bp, synthetic construct
  misc_feature 1..36: note = Artificial
  source 1..36: mol_type = other DNA
atcgaatatatattgccacgtggccaaagggtcggc

SEQ ID NO: 21 - DNA, 130 bp, synthetic construct
  misc_feature 1..130: note = Artificial
  source 1..130: mol_type = other DNA
cgagagagccgtctccctcttcgcttctcctctcccccccgtgatctgacgccgacgccacgtggcaagccgcctatatacgccggcgacgaccccctcccaccacccgccgccgccgccgcgcccccgc

SEQ ID NO: 22 - DNA, 42 bp, synthetic construct
  misc_feature 1..42: note = Artificial
  source 1..42: mol_type = other DNA
acttaatgtctcgccgccgcgggaaaagccgacgcgggaaaa

SEQ ID NO: 23 - DNA, 42 bp, synthetic construct
  misc_feature 1..42: note = Artificial
  source 1..42: mol_type = other DNA
aagtttttcccgcgtcggcttttcccgcggcggcgagacatt

SEQ ID NO: 24 - DNA, 136 bp, synthetic construct
  misc_feature 1..136: note = Artificial
  source 1..136: mol_type = other DNA
cggcggcgtcgtcgtcttcctcctcttccccccgagttggattcgtggcctgtctcgccgccgcgggaaaagccgacgcgggaaaacggcggatgggaggggaggagggaatggcggcggggaggaagaagcgggt

SEQ ID NO: 25 - DNA, 136 bp, synthetic construct
  misc_feature 1..136: note = Artificial
  source 1..136: mol_type = other DNA
gatacaggtagagagaggtgagaaggcagtggtgggagatccatcttgtctcgccgccgcgggaaaagccgacgcgggaaaagcgaggtgcctccatggctgagaaggagggcaacctcgacgccgtcctcaagga

SEQ ID NO: 26 - DNA, 39 bp, synthetic construct; identical to SEQ ID NO: 15
  misc_feature 1..39: note = Artificial
  source 1..39: mol_type = other DNA

SEQ ID NO: 27 - DNA, 39 bp, synthetic construct
  misc_feature 1..39: note = Artificial
  source 1..39: mol_type = other DNA
gagcgtcggcggcggcgtcggcctttcccgcgtcggctt

SEQ ID NO: 28 - DNA, 124 bp, synthetic construct
  misc_feature 1..124: note = Artificial
  source 1..124: mol_type = other DNA
acctgccactcgcccactcctcctcctcccccatcctccagtgcctttgtatatagccgacgccgacgccgccctacggtgtcacgcagccatggcgctccgcacgctggcctcgaggaaaacc

SEQ ID NO: 29 - DNA, 36 bp, synthetic construct
  misc_feature 1..36: note = Artificial
  source 1..36: mol_type = other DNA
ctgcaacctttgtatatagccgacgccgacgccgcc

SEQ ID NO: 30 - DNA, 36 bp, synthetic construct
  misc_feature 1..36: note = Artificial
  source 1..36: mol_type = other DNA
gcagggcggcgtcggcgtcggctatatacaaaggtt

SEQ ID NO: 31 - DNA, 36 bp, synthetic construct
  misc_feature 1..36: note = Artificial
  source 1..36: mol_type = other DNA
cctttgtatatagccgacgccgacgccgccgggcga

SEQ ID NO: 32 - DNA, 36 bp, synthetic construct
  misc_feature 1..36: note = Artificial
  source 1..36: mol_type = other DNA
ccggcggcgtcggcgtcggctatatacaaaggtcgc

SEQ ID NO: 33 - DNA, 36 bp, synthetic construct
  misc_feature 1..36: note = Artificial
  source 1..36: mol_type = other DNA
atgcaatatatattgccacgtggccaaagggtcggc

SEQ ID NO: 34 - DNA, 34 bp, synthetic construct
  misc_feature 1..34: note = Artificial
  source 1..34: mol_type = other DNA
aaaagccgacgccacgtggcaagccgcctatata

SEQ ID NO: 35 - DNA, 34 bp, synthetic construct
  misc_feature 1..34: note = Artificial
  source 1..34: mol_type = other DNA
tttttatataggcggcttgccacgtggcgtcggc

SEQ ID NO: 36 - DNA, 42 bp, synthetic construct
  misc_feature 1..42: note = Artificial
  source 1..42: mol_type = other DNA
cactaatgtctcgccgccgcgggaaaagccgacgcgggaaaa

SEQ ID NO: 37 - DNA, 42 bp, synthetic construct
  misc_feature 1..42: note = Artificial
  source 1..42: mol_type = other DNA
agtgttttcccgcgtcggcttttcccgcggcggcgagacatt

SEQ ID NO: 38 - DNA, 40 bp, synthetic construct
  misc_feature 1..40: note = Artificial
  source 1..40: mol_type = other DNA
gtattgtctcgccgccgcgggaaaagccgacgcgggaaaa

SEQ ID NO: 39 - DNA, 40 bp, synthetic construct
  misc_feature 1..40: note = Artificial
  source 1..40: mol_type = other DNA
atacttttcccgcgtcggcttttcccgcggcggcgagaca

SEQ ID NO: 40 - DNA, 40 bp, synthetic construct
  misc_feature 1..40: note = Artificial
  source 1..40: mol_type = other DNA
ttattgtctcgccgccgcgggaaaagccgacgcgggaaaa

SEQ ID NO: 41 - DNA, 40 bp, synthetic construct
  misc_feature 1..40: note = Artificial
  source 1..40: mol_type = other DNA
ataattttcccgcgtcggcttttcccgcggcggcgagaca

SEQ ID NO: 42 - DNA, 39 bp, synthetic construct; identical to SEQ ID NO: 15
  misc_feature 1..39: note = Artificial
  source 1..39: mol_type = other DNA

SEQ ID NO: 43 - DNA, 39 bp, synthetic construct; identical to SEQ ID NO: 27
  misc_feature 1..39: note = Artificial
  source 1..39: mol_type = other DNA

SEQ ID NO: 44 - DNA, 34 bp, synthetic construct
  misc_feature 1..34: note = Artificial
  source 1..34: mol_type = other DNA
acgcggcggcgtcggcgtcggctatatacaaagg

SEQ ID NO: 45 - DNA, 34 bp, synthetic construct
  misc_feature 1..34: note = Artificial
  source 1..34: mol_type = other DNA
gcgtcctttgtatatagccgacgccgacgccgcc

SEQ ID NO: 46 - DNA, 36 bp, synthetic construct; identical to SEQ ID NO: 29
  misc_feature 1..36: note = Artificial
  source 1..36: mol_type = other DNA

SEQ ID NO: 47 - DNA, 36 bp, synthetic construct; identical to SEQ ID NO: 30
  misc_feature 1..36: note = Artificial
  source 1..36: mol_type = other DNA

SEQ ID NO: 48 - DNA, 36 bp, synthetic construct
  misc_feature 1..36: note = Artificial
  source 1..36: mol_type = other DNA
cgtgcctttgtatatagccgacgccgacgccgccgg

SEQ ID NO: 49 - DNA, 40 bp, synthetic construct
  misc_feature 1..40: note = Artificial
  source 1..40: mol_type = other DNA
cacgccggcggcgtcggcgtcggctatatacaaaggtcgc

SEQ ID NO: 50 - DNA, 25 bp, synthetic construct
  misc_feature 1..25: note = Artificial
  source 1..25: mol_type = other DNA
tgcaattggagccatcttttctgcg

SEQ ID NO: 51 - DNA, 24 bp, synthetic construct
  misc_feature 1..24: note = Artificial
  source 1..24: mol_type = other DNA
aatgcctgcggatagtggttaccc

SEQ ID NO: 52 - DNA, 26 bp, synthetic construct
  misc_feature 1..26: note = Artificial
  source 1..26: mol_type = other DNA
tgttacattccctcaggtgcttcgtg

SEQ ID NO: 53 - DNA, 26 bp, synthetic construct
  misc_feature 1..26: note = Artificial
  source 1..26: mol_type = other DNA
tgtcaccagcaacctttcgaacatca

SEQ ID NO: 54 - DNA, 25 bp, synthetic construct
  misc_feature 1..25: note = Artificial
  source 1..25: mol_type = other DNA
tggcaattggaaaagaaacagggcg

SEQ ID NO: 55 - DNA, 25 bp, synthetic construct
  misc_feature 1..25: note = Artificial
  source 1..25: mol_type = other DNA
tgggaatgtagggctatccactggt

SEQ ID NO: 56 - DNA, 25 bp, synthetic construct
  misc_feature 1..25: note = Artificial
  source 1..25: mol_type = other DNA
ccaaaatcctcatcaatggcccagc

SEQ ID NO: 57 - DNA, 23 bp, synthetic construct
  misc_feature 1..23: note = Artificial
  source 1..23: mol_type = other DNA
actgggccacactacaatcctgc

SEQ ID NO: 58 - DNA, 25 bp, synthetic construct
  misc_feature 1..25: note = Artificial
  source 1..25: mol_type = other DNA
actacaactcacaggatgcagcagc

SEQ ID NO: 59 - DNA, 40 bp, synthetic construct
  misc_feature 1..40: note = Artificial
  source 1..40: mol_type = other DNA
atagcacaatgccgaccctttggccacgtggcaatatata

SEQ ID NO: 60 - DNA, 40 bp, synthetic construct
  misc_feature 1..40: note = Artificial
  source 1..40: mol_type = other DNA
ctattatatattgccacgtggccaaagggtcggcattgtg

SEQ ID NO: 61 - DNA, 40 bp, synthetic construct
  misc_feature 1..40: note = Artificial
  source 1..40: mol_type = other DNA
tcggcacaatgccgacgccacgtggcaagccgcctatata

SEQ ID NO: 62 - DNA, 40 bp, synthetic construct
  misc_feature 1..40: note = Artificial
  source 1..40: mol_type = other DNA
ccgatatataggcggcttgccacgtggcgtcggcattgtg

SEQ ID NO: 63 - DNA, 70 bp, synthetic construct
  misc_feature 1..70: note = Artificial
  source 1..70: mol_type = other DNA
ctcttgacgcacaatcccacttgtctctatatagccgccgcgggaaaagccgacgcgggaaaatatataa

SEQ ID NO: 64 - DNA, 65 bp, synthetic construct
  misc_feature 1..65: note = Artificial
  source 1..65: mol_type = other DNA
agagttatatattttcccgcgtcggcttttcccgcggcggctatatagagacaagtgggattgtg

SEQ ID NO: 65 - DNA, 73 bp, synthetic construct
  misc_feature 1..73: note = Artificial
  source 1..73: mol_type = other DNA
gtaactgacgcacaatcccacttgtctctatatagccgccgcgggaaaagccgacgcgggaaaatatataagc

SEQ ID NO: 66 - DNA, 73 bp, synthetic construct
  misc_feature 1..73: note = Artificial
  source 1..73: mol_type = other DNA
ttacgcttatatattttcccgcgtcggcttttcccgcggcggctatatagagacaagtgggattgtgcgtcag

SEQ ID NO: 67 - DNA, 64 bp, synthetic construct
  misc_feature 1..64: note = Artificial
  source 1..64: mol_type = other DNA
catccacaatcccactacctcgtgtctcgccgccgcgggaaaagccgacgcgggaaaatatata

SEQ ID NO: 68 - DNA, 64 bp, synthetic construct
  misc_feature 1..64: note = Artificial
  source 1..64: mol_type = other DNA
gatgtatatattttcccgcgtcggcttttcccgcggcggcgagacacgaggtagtgggattgtg

SEQ ID NO: 69 - DNA, 55 bp, synthetic construct
  misc_feature 1..55: note = Artificial
  source 1..55: mol_type = other DNA
acaccacaatacctcgtgtctcgccgacgcgggaaaggccgacgccgccgccgac

SEQ ID NO: 70 - DNA, 55 bp, synthetic construct
  misc_feature 1..55: note = Artificial
  source 1..55: mol_type = other DNA
gtgtgtcggcggcggcgtcggcctttcccgcgtcggcgagacacgaggtattgtg

SEQ ID NO: 71 - DNA, 58 bp, synthetic construct
  misc_feature 1..58: note = Artificial
  source 1..58: mol_type = other DNA
atgacacaatcccactcctttgtatatagccgacgccgacgccgccacctcgtgtctc

SEQ ID NO: 72 - DNA, 58 bp, synthetic construct
  misc_feature 1..58: note = Artificial
  source 1..58: mol_type = other DNA
tcatgagacacgaggtggcggcgtcggcgtcggctatatacaaaggagtgggattgtg

SEQ ID NO: 73 - DNA, 58 bp, synthetic construct
  misc_feature 1..58: note = Artificial
  source 1..58: mol_type = other DNA
tctccacaatcccactcctttgtatatagccgacgccgacgccgccacctcgtgtctc

SEQ ID NO: 74 - DNA, 58 bp, synthetic construct
  misc_feature 1..58: note = Artificial
  source 1..58: mol_type = other DNA
gagagagacacgaggtggcggcgtcggcgtcggctatatacaaaggagtgggattgtg

SEQ ID NO: 75 - DNA, 58 bp, synthetic construct
  misc_feature 1..58: note = Artificial
  source 1..58: mol_type = other DNA
acgccacaatcccactcctttgtatatagccgacgccgacgccgccacctcgtgtctc

SEQ ID NO: 76 - DNA, 58 bp, synthetic construct
  misc_feature 1..58: note = Artificial
  source 1..58: mol_type = other DNA
gcgtgagacacgaggtggcggcgtcggcgtcggctatatacaaaggagtgggattgtg

SEQ ID NO: 77 - DNA, 70 bp, synthetic construct
  misc_feature 1..70: note = Artificial
  source 1..70: mol_type = other DNA
tttccctcgtgtctcatgacgcacaatcccactcctttgtatatagccgacgccgacgccgccgtatata

SEQ ID NO: 78 - DNA, 70 bp, synthetic construct
  misc_feature 1..70: note = Artificial
  source 1..70: mol_type = other DNA
gaaatatatacggcggcgtcggcgtcggctatatacaaaggagtgggattgtgcgtcatgagacacgagg

SEQ ID NO: 79 - DNA, 10 bp, synthetic construct
  misc_feature 1..10: note = Artificial
  source 1..10: mol_type = other DNA
gccacgtggc

SEQ ID NO: 80 - DNA, 11 bp, synthetic construct
  misc_feature 1..11: note = Artificial
  source 1..11: mol_type = other DNA
taagagccgcc

SEQ ID NO: 81 - DNA, 11 bp, synthetic construct
  misc_feature 1..11: note = Artificial
  source 1..11: mol_type = other DNA
cctcgtgtctc

SEQ ID NO: 82 - DNA, 36 bp, synthetic construct
  misc_feature 1..36: note = Artificial
  source 1..36: mol_type = other DNA
cacaatgccgaccctttggccacgtggcaatatata

SEQ ID NO: 83 - DNA, 36 bp, synthetic construct
  misc_feature 1..36: note = Artificial
  source 1..36: mol_type = other DNA
cacaatgccgacgccacgtggcaagccgcctatata

SEQ ID NO: 84 - DNA, 67 bp, synthetic construct
  misc_feature 1..67: note = Artificial
  source 1..67: mol_type = other DNA
ctgacgcacaatcccacttgtctctatatagccgccgcgggaaaagccgacgcgggaaaatatataa

SEQ ID NO: 85 - DNA, 67 bp, synthetic construct; identical to SEQ ID NO: 84
  misc_feature 1..67: note = Artificial
  source 1..67: mol_type = other DNA

SEQ ID NO: 86 - DNA, 60 bp, synthetic construct
  misc_feature 1..60: note = Artificial
  source 1..60: mol_type = other DNA
cacaatcccactacctcgtgtctcgccgccgcgggaaaagccgacgcgggaaaatatata

SEQ ID NO: 87 - DNA, 51 bp, synthetic construct
  misc_feature 1..51: note = Artificial
  source 1..51: mol_type = other DNA
cacaatacctcgtgtctcgccgacgcgggaaaggccgacgccgccgccgac

SEQ ID NO: 88 - DNA, 54 bp, synthetic construct
  misc_feature 1..54: note = Artificial
  source 1..54: mol_type = other DNA
cacaatcccactcctttgtatatagccgacgccgacgccgccacctcgtgtctc

SEQ ID NO: 89 - DNA, 54 bp, synthetic construct; identical to SEQ ID NO: 88
  misc_feature 1..54: note = Artificial
  source 1..54: mol_type = other DNA

SEQ ID NO: 90 - DNA, 54 bp, synthetic construct; identical to SEQ ID NO: 88
  misc_feature 1..54: note = Artificial
  source 1..54: mol_type = other DNA

SEQ ID NO: 91 - DNA, 66 bp, synthetic construct
  misc_feature 1..66: note = Artificial
  source 1..66: mol_type = other DNA
cctcgtgtctcatgacgcacaatcccactcctttgtatatagccgacgccgacgccgccgtatata

SEQ ID NO: 92 - protein, 231 aa, Oryza sativa
  source 1..231: note = subsp. japonica
MALRTLASRKTLAAAALPLAAAAAARGVTTVALPDLPYDYGALEPAISGEIMRLHHQKHHATYVANYNKALEQLDAAVAKGDAPAIVHLQSAIKFNGGGHVNHSIFWNNLKPISEGGGDPPHAKLGWAIDEDFGSFEALVKKMSAEGAALQGSGWVWLALDKEAKKLSVETTANQDPLVTKGANLVPLLGIDVWEHAYYLQYKNVRPDYLSNIWKVMNWKYAGEVYENATA

SEQ ID NO: 93 - protein, 164 aa, Oryza sativa
  source 1..164: note = subsp. japonica
MAGKAGGLKGVALIGGAGGNSAVAGALHFFQDPSTGYTEVRGRVTGLAPGLHGFHIHSFGDTTNGCNSTGPHFNPHNKSHGAPSDDERHVGDLGNIVANKDGVADIFIKDLQISLSGPHSILGRAVVVHADSDDLGRGGHELSKTTGNAGARIGCGIIGLRSAV

SEQ ID NO: 94 - protein, 60 aa, Oryza sativa
  source 1..60: note = subsp. japonica
MVKAVVVLGSSEIVKGTIHFVQEGDGPTTVTGSVSGLKPGLHGFHIHALGDTTNGCMSTG

SEQ ID NO: 95 - protein, 1148 aa, Oryza sativa
  source 1..1148: note = subsp. japonica
MDNPEAEPDDAVLFVGVSLVLGIASRHLLRGTRVPYTVALLVLGVALGSLEFGTKHGMGKLGAGIRIWANINPDLLLAVFLPALLFESSFSMEIHQIKKCMAQMVLLAGPGVLISTFFLGSALKLTFPYNWNWKTSLLLGGLLSATDPVAVVALLKELGASKKLSTIIEGESLMNDGTAIVVYQLFYRMVLGRTFDAGSIIKFLSEVSLGAVALGLAFGIASVLWLGFIFNDTIIEIALTLAVSYIAFFTAQDALEVSGVLTVMTLGMFYAAFAKTAFKGDSQQSLHHFWEMVAYIANTLIFILSGVVIADGVLENNVHFERHGASWGFLLLLYVFVQISRILVVVILYPLLRHFGYGLDLKEATILVWAGLRGAVALSLSLSVKRASDAVQTHLKPVDGTMFVFFTGGIVFLTLIFNGSTTQFLLHLLGMDRLAATKLRILNYTKYEMLNKALEAFGDLRDDEELGPPADWVTVKKYITCLNDLDDEPVHPHAVSDRNDRMHTMNLRDIRVRLLNGVQAAYWGMLEEGRITQTTANILMRSVDEAMDLVPTQELCDWKGLRSNVHFPNYYRFLQMSRLPRRLITYFTVERLESGCYICAAFLRAHRIARRQLHDFLGDSEVARIVIDESNAEGEEARKFLEDVRVTFPQVLRVLKTRQVTYSVLTHLSEYIQNLQKTGLLEEKEMAHLDDALQTDLKKFKRNPPLVKMPRVSDLLNTHPLVGALPAAMRDPLLSSTKETVKGHGTILYREGSRPTGIWLVSIGVVKWTSQRLSSRHSLDPILSHGSTLGLYEVLIGKPYICDMITDSVVHCFFIEAEKIEQLRQSDPSIEIFLWQESALVVARLLLPMMFEKMATHELRVLITERSTMNIYIKGEEIELEQNFIGILLEGFLKTKNQTLITPPGLLLPPNADLNLFGLESSAINRIDYCYTAPSYQVEARARILFVEIGRPEIEADLQRSASLISQTLELPRTQSKEHSGLLSWPESFRKSRGAQNGASLTEIRDHPASFSARALQLSMYGSMINDMKSGQGQGQRRQRHRHTKASSNKAHSSSYPRVPSRSSNTQRPLLSVQSEGANMTTARQAAAAGASLPPEPEEAGRRRRRQRKAIEEDEDNSSDESAGEEVIVRVDSPSMLTFRQPSSAADR

SEQ ID NO: 96 - protein, 1142 aa, Oryza sativa
  source 1..1142: note = subsp. japonica
MDNPEAEPDDAVLFVGVSLVLGIASRHLLRGTRVPYTVALLVLGVALGSLEFGTKHGMGKLGAGIRIWANINPDLLLAVFLPALLFESSFSMEIHQIKKCMAQMVLLAGPGVLISTFFLGSALKLTFPYNWNWKTSLLLGGLLSATDPVAVVALLKELGASKKLSTIIEGESLMNDGTAIVVYQLFYRMVLGRTFDAGSIIKFLSEVSLGAVALGLAFGIASVLWLGFIFNDTIIEIALTLAVSYIAFFTAQDALEVSGVLTVMTLGMFYAAFAKTAFKGDSQQSLHHFWEMVAYIANTLIFILSGVVIADGVLENNVHFERHGASWGFLLLLYVFVQISRILVVVILYPLLRHFGYGLDLKEATILVWAGLRGAVALSLSLSVKRASDAVQTHLKPVDGTMFVFFTGGIVFLTLIFNGSTTQFLLHLLGMDRLAATKLRILNYTKYEMLNKALEAFGDLRDDEELGPPADWVTVKKYITCLNGLDDEPVHPHAVSDRNDRMHTMNLRDIRVRLLNGVQAAYWGMLEEGRITQTTANILMRSVDEAMDLVPTQELCDWKGLRSNVHFPNYYRFLQMSRLPRRLITYFTVERLESGCYICAAFLRAHRIARRQLHDFLGDSEVARIVIDESNAEGEEARKFLEDVRVTFPQVLRVLKTRQVTYSVLTHLSEYIQNLQKTGLLEEKEMAHLDDALQTDLKKFKRNPPLVKMPRVSDLLNTHPLVGALPAAMRDPLLSSTKETVKGHGTILYREGSRPTGIWLVSIGVVKWTSQRLSSRHSLDPILSHGSTLGLYEVLIGKPYICDMITDSVVHCFFIEAEKIEQLRQSDPSIEIFLWQESALVVARLLLPMMFEKMATHELRVLITERSTMNIYIKGEEIELEQNFIGILLEGFLKTKNQTLITPPGLLLPPNADLNLFGLESSAINRIDYCYTAPSYQVEARARILFVEIGRPEIEADLQRSASLISQTLELPRTQSKEHSETIQQLSGAQNGASLTEIRDHPASFSARALQLSMYGSMINDMKSGQGQGQRRQRHRHTKASSNKAHSSSYPRVPSSSSNTQRPLLSVQSEGANMTTARQAAAAGASLPPEPEEAGRRRRRQRKAIEEDEDNSSDESAGEEVIVRVDSPSMLTFRQPSSAADR

SEQ ID NO: 97 - protein, 535 aa, Oryza sativa
  source 1..535: note = subsp. japonica
MGMEVAAARLGALYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIGAIFSATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKITWRQQVVIWWAGLMRGAVSIALAYNKFTRSGHTQLHGNAIMITSTITVVLFSTMVFGMMTKPLIRLLLPASGHPVTSEPSSPKSLHSPLLTSMQGSDLESTTNIVRPSSLRMLLTKPTHTVHYYWRKFDDALMRPMFGGRGFVPFSPGSPTEQSHGGR

SEQ ID NO: 98 - protein, 535 aa, Oryza sativa
  source 1..535: note = subsp. japonica
MGMEVAAARLGPLYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIRAIFPATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKRTWRQQVVIWWAGLMRGAVSIALAYNKFTRSGHTQLHGNAIMITSTITVVLWSTMVFGMMTKPLIRLLLPASGHPVTSEPSSPKSLHSPLLTRMQGSDLESTTNIVRPSSLRMLLTKPTHTVHYYWRKFDDALMRPMFGGRGFVPFSPGSPTEQSHGGR

SEQ ID NO: 99 - protein, 530 aa, Oryza sativa
  source 1..530: note = subsp. japonica
MTSIYHDFIHNKLQSFGRIGRYFVNFVVLAHRFIALHIHPFWIQLSYFLLISILGSVLLMFLKPSNPEFRPGYIDMLFLSTSALTLSSLITIEMEVLSSSQIVVITLLMLLGGEVFVSFLGLMLRLNHKHNPEFSGDKVSSVPIELDTINSASTVISCEELQLEAAIPEVPSSTIKDLKRSKRLRWFLGFVVFSYFVVIHVAGFLLVLWYISRVSSAKAPLKKKGINIALFSFSVTVSSFANVGLVPTNENMAIFSKNPGLLLLFIGQILAGNTLYPLFLRLLIWFLGKVTKLRELKLMIKNPEELQYDYLLPKLPTAFLASTVIGLMASLVTLFGAVDWNSSVFDGLSSYQKIINALFMAVNARHSGENSIDCSLIAPAVLVLFIILMYLPPSTTFALSNGDEKTANKKAKRKLGLVVQNLAFSQLACISVFVIVAFITERSRLRNDPLNFSALNMIFEIISAYGNVGLSTGYSCSRLQKLHPGSICQDKPYSLSGWWSDEGKLLLVFVMLYGRLKAFTKGTGEYWRLW

SEQ ID NO: 100 - protein, 300 aa, Oryza sativa
  source 1..300: note = subsp. japonica
MGGEEGMAAGRKKRVGRYEVGRTIGQGTFAKVKFAVDADTGAAVAMKVLDKDTILNHRMLHQIKREISIMKIVRHPNIVRLNEVLAGKTKIYIILELITGGELFDKIARQGKLRENEARKYFQQLIDAINYCHSKGVYHRDLKPENLLLDSRGNLKVSDFGLSTLAQKGVGLLHTTCGTPNYVAPEVLSNNGYDGSAADVWSCGVILYVLMAGYLPFEEDDLPTLYDKITAGQFSCPYWFSPGATSLIHRILDPNPKTRITIEQIREDTWFKKTYVAIKRGEDENVDLDDVQAVFDNIED

SEQ ID NO: 101 - protein, 956 aa, Oryza sativa
  source 1..956: note = subsp. japonica
MAEDKGGLDAVLKESVDLENIPIEEVFQNLKCCRQGLTSEEAQLRLQLFGPNKLEEKEESKFLKFLGFMWNPLSWVMEAAAIMAIALANGGGKPPDWQDFVGIITLLLINSTISFIEENNAGNAAAALMARLAPKAKVLRNGSWTEEEAAILVPGDIISIKLGDIIPADARLLEGDPLKIDQSALTGESLPATKGPGDGVYSGSTVKQGEIEAVVIATGVHTFFGKAAHLVDSTNQVGHFQKVLTAIGNFCICSIAVGMFVEIIVMYPIQHRPYRPGIDNLLVLLIGGIPIAMPTVLSVTMAIGSHRLSQQGAITKRMTAIEEMAGMDVLCSDKTGTLTLNKLTVDKNLIEIFERGVTQDQVILMAARASRTENQDAIDTAIVGMLADPKEARAGIQEVHFLPFNPTDKRTALTYIDSDGKMYRVSKGAPEQILNLAHNKTQIERRVHAVIDKFAERGLRSLAVAYQEVPDGRKESPGGPWRFVALLPLFDPPRHDSAETIRRALNLGVNVKMITGDQLAIGKETGRRLGMGTNMYPSSALLGQNKDESVAALPVDDLIEKADGFAGVFPEHKYEIVKRLQARKHICGMTGDGVNDAPALKKADIGIAVADATDAARSASDIVLTEPGLSVIISAVLTSRAIFQRMKNYTIYAVSITIRIVFGFMLLALIWEFDFPPFMVLIIAILNDGTIMTISKDLVKPSPLPDSWKLAEIFTTGVVLGGYLAMMTVIFFWAAYKTNFFPRIFHVESLEKTAQDDYQKLASAVYLQVSTISQALIFVTRSRSWSFIERPGFLLVFAFFVAQLIATLIAVYANWAFTSIKGIGWGWAGIVWLYNLVFYFPLDIIKFLIRYALSGKAWDLVIEQRIAFTRKKDFGKEERELKWAHAHRTLHGLQPPDAKPFPEKTGYSELNQMAEEAKRRAEIARLRELHTLKGHVESVVKLKGLDIDTIHQSYTV

SEQ ID NO: 102 - protein, 620 aa, Oryza sativa
  source 1..620: note = subsp. japonica
MSYDRVTTFEDSEKESEYGYVRKVSGPVVVADGMGGAAMYELVRVGNDNLIGEIIRLEGDSATIQVYEETAGLMVNDPVLRTRKPLSVELGPGILGNIFDGIQRPLKTIAIKSGDVYIPRGVSVPALDKDQLWDFEPKKLGVGDAITGGDLYATVFENTLMKHHVALPPGSMGKISYIAPAGQYSLQDTVLELEFQGIKKQFTMLQTWPVRSPRPVSSKLAADTPLLTGQRVLDALFPSVLGGTCAIPGAFGCGKTVISQALSKYSNSEAVVYVGCGERGNEMAEVLMDFPQLTMTLPDGREESVMKRTTLVANTSNMPVAAREASIYTGITIAEYFRDMGYNVSMMADSTSRWAEALREISGRLAEMPADSGYPAYLAARLASFYERAGKVKCLGSPDRTGSVTIVGAVSPPGGDFSDPVTSATLSIVQVFWGLDKKLAQRKHFPSVNWLISYSKYSKALESFYEKFDQDFIDIRTKAREVLQREDDLNEIVQLVGKDALAESDKITLETAKLLREDYLAQNAFTPYDKFCPFYKSVWMMRNIIHFNTLANQAVERAANADGQKITYSVIKHRMGDLFYRLVSQKFEDPAEGEDVLVAKFQKLYDDLTTGFRNLEDEAR

SEQ ID NO: 103 - protein, 488 aa, Oryza sativa
MGLVKDGAADLEEGTLEIGMEYRTVSGVAGPLVILDKVKGPKYQEIVNIRLGDGTTRRGQVLEVDGEKAVVQVFEGTSGIDNKYTTVQFTGEVLKTPVSLDMLGRIFNGSGKPIDNGPPILPEAYLDISGSSINPSERTYPEEMIQTGISTIDVMNSIARGQKIPLFSAAGLPHNEIAAQICRQAGLVKSLEKGKHAEGGEDDNFAIVFAAMGVNMETAQFFKRDFEENGSMERVTLFLNLANDPTIERIITPRIALTTAEYLAYECGKHVLVILTDMSSYADALREVSAAREEVPGRRGYPGYMYTDLATIYERAGRIEGRSGSITQIPILTMPNDDITHPTPDLTGYITEGQIYIDRQLHNRQIYPPINVLPSLSRLMKSAIGEGMTRRDHSDVSNQLYANYAIGKDVQAMKAVVGEEALSSEDLLYLEFLDKFERKFVTQGAYDTRNIFQSLDLAWTLLRIFPRELLHRIPAKTLDQYYSRDATH

SEQ ID NO: 104 - protein, 328 aa, Leymus chinensis
MAASLQAAATLMPAKIGGRASSARPSSHVARAFGVDAGARITCSLQSDIREVASKCAEAAKMAGFALATSALLVSGASAEGAPKRLTFDEIQSKTYMEVKGTGTANQCPTIDGGVDSFPFKAGKYEMKKFCLEPTSFTVKAEGIQKNEPPAFQKTKLMTRLTYTLDEMEGPLEVGADGTLKFEEKDGIDYAAVTVQLPGGERVPFLFTVKQLVATGKPESFSGPFLVPSYRGSSFLDPKGRGGSTGYDNAVALPAGGRGDEEELAKENVKNASSSTGNITLSVTKSKPETGEVIGVFERVQPSDTDLGAKAPKDVKIQGVWYAQLESN

SEQ ID NO: 105 - protein, 142 aa, Oryza sativa
  source 1..142: note = subsp. japonica
VVNASSSEASSDEKNVTRRRLALLGAGALATGLLKSSSAYAEEVPKNYKSYVDSKDGYSYLYPADWRDFDFLGHDSAFKDRNVALQCVRVGFIPTTKTDIRDLGPMDEAIFNLVNNVYAAPNQIPTVYDMQERTVHGKNYWT

SEQ ID NO: 106 - protein, 217 aa, Oryza sativa
  source 1..217: note = subsp. indica
MAQAMASMTGLSQGVQLPAGPRRAGGRSRLAVVRADAAAADVQTGRRAVLGLVATGIAGGALAQAALAEAAKPIKLGPPPPPSGGLPGTLNSDQARDTDLPLRERFYLQPLPPAEAAARAKESAQDIINLKPLIEKKQWPFVRDDLRLRASYLRYDLKTVINSKPKDEKKGLKDLTGKLFATIDGLDHAAKIKSPEEAEKYYTLTKSALGDVLAKLG

SEQ ID NO: 107 - protein, 505 aa, Oryza sativa
  source 1..505: note = subsp. japonica
MEGAGRDGNPLGGYRIGKTLGIGSFGKVKIAEHILTGHKVAIKILNRRKIKSMEMEEKVKREIKILRLFMHPHIIRLYEVIDTPADIYVVMEYVKSGELFDYIVEKGRLQEEEARRFFQQIISGVEYCHRNMVVHRDLKPENLLLDSKCNVKIADFGLSNVMRDGHFLKTSCGSPNYAAPEVISGKLYAGPEVDVWSCGVILYALLCGTLPFDDENIPNLFKKIKGGIYTLPSHLSPLARDLIPRMLVVDPMKRITIREIREHQWFTVGLPRYLAVPPPDTAQQVKKLDDETLNDVINMGFDKNQLIESLHKRLQNEATVAYYLLLDNRLRTTSGYLGAEFHESMESSLAQVTPAETPNSATDHRQHGHMESPGFGLRHHFAADRKWALGLQSRAHPREIITEVLKALQELNVCWKKIGHYNMKCRWSPSFPSHESMMHNNHGFGAESAIIETDDSEKSTHTVKFEIQLYKTRDEKYLLDLQRVSGPQLLFLDLCSAFLTQLRVL

SEQ ID NO: 108 - protein, 505 aa, Oryza sativa; identical to SEQ ID NO: 107
  source 1..505: note = subsp. japonica

SEQ ID NO: 109 - protein, 503 aa, Oryza sativa
MEGAGSDGNPLGGYRIGKTLGIGSFGKVKIAEHILTGHKVAIKILNRRKSMEMEEKVKREIKILRLFMHPHIIRLYEVIDTPADIYVVMEYVKSGELFDYDVEKGRLQEEEARRFFQQIISGVEYCHRNMVVRRDLKPENLLLDSKCNVKIADFGLSNVMRDGHFLKTSCGSPNYAAPEVISGKLYAGPEVDVWSCGVILYALLCGTLPFDDENIPNLFKNIKGGIYTLPSHLSPLGRDLIPRMLVVDPMKRITIREIREHQWFTVGLPRYLAVPPPDTAQQVKKLDDETQNDVINMGFDKNQLIESLHKRLQNEATVAYYLLLDNRLRTTSGYLGAEFHESMVSSLAQVTPAETPNSATDHRQHGHMESPGFGLRHHFAADRKWALGLQSRAHPREIITEVLKALQELNVCWKKIGHHNMKCRWSPSFPSHESMMHNNHGFGAASAMIETDDSEKSTHTVKFEIQLYKTRDEKYFLDLQRLSGPQLLFLDLCSAFLTQLRVL

SEQ ID NO: 110 - protein, 505 aa, Oryza sativa; identical to SEQ ID NO: 107
  source 1..505: note = subsp. japonica

SEQ ID NO: 111 - DNA, 36 bp, synthetic construct; identical to SEQ ID NO: 19
  misc_feature 1..36: note = Artifical
  source 1..36: mol_type = other DNA

SEQ ID NO: 112 - protein, 497 aa, Arabidopsis thaliana
MAMLGFYVTFIFFLVCLFTYFFLQKKPQGQPILKNWPFLRMLPGMLHQIPRIYDWTVEVLEATNLTFYFKGPWLSGTDMLFTADPRNIHHILSSNFGNYPKGPEFKKIFDVLGEGILTVDFELWEEMRKSNHALFHNQDFIELSVSSNKSKLKEGLVPFLDNAAQKNIIIELQDVFQRFMFDTSSILMTGYDPMSLSIEMLEVEFGEAADIGEEAIYYRHFKPVILWRLQNWIGIGLERKMRTALATVNRMFAKIISSRRKEEISRAKTEPYSKDALTYYMNVDTSKYKLLKPNKDKFIRDVIFSLVLAGRDTTSSVLTWFFWLLSKHPQVMAKLRHEINTKFDNEDLEKLVYLHAALSESMRLYPPLPFNHKSPAKPDVLPSGHKVDANSKIVICIYALGRMRSVWGEDALDFKPERWISDNGGLRHEPSYKFMAFNSGPRTCLGKNLALLQMKMVALEIIRNYDFKVIEGHKVEPIPSILLRMKHGLKVTVTKKI

SEQ ID NO: 113 - protein, 1084 aa, Oryza sativa
MAGNEWINGYLEAILDSGGAAGGGGGGGGGGGGGGGGGGGGGGGGVDPRSPAAGAASPRGPHMNFNPTHYFVEEVVKGVDESDLHRTWIKVVATRNARERSTRLENMCWRIWHLARKKKQLELEGILRISARRKEQEQVRRETSEDLAEDLFEGEKADTVGELAQQDTPMKKKFQRNFSELTVSWSDENKEKKLYIVLISLHGLVRGDNMELGRDSDTGGQVKYVVELARALAMMPGVYRVDLFTRQVSSPEVDWSYGEPTEMLTSGSTDGEGSGESAGAYIVRIPCGPRDKYLRKEALWPYLQEFVDGALAHILNMSKALGEQVSNGKLVLPYVIHGHYADAGDVAALLSGALNVPMVLTGHSLGRNKLEQIMKQGRMSKEEIDSTYKIMRRIEGEELALDAAELVITSTRQEIDEQWGLYDGFDVKLEKVLRARARRGVSCHGRFMPRMVVIPPGMDFSSVVVPEDTSDGDDGKDFEIASPRSLPPIWAEVMRFLTNPHKPMILALSRPDPKKNITTLVKAFGECRPLRELANLILIMGNRDDIDEMSAGNASVLTTVLKLIDKYDLYGSVAFPKHHKQSDVPEIYRLTGKMKGVFINPALVEPFGLTLIEAAAHGLPIVATKNGGPVDIKNALNNGLLVDPHDQHAIADALLKLVADKNLWQECRKNGLRNIQLYSWPEHCRTYLTRIAGCRIRNPRWLMDTPADAAAEEEEALEDSLMDVQDLSLRLSIDGERGSSMNDAPSSDPQDSVQRIMNKIKRSSPADTDGAKIPAEAAATATSGAMNKYPLLRRRRRLFVIAVDCYGDDGSASKRMLQVIQEVFRAVRSDSQMSRISGFALSTAMPLPETLKLLQLGKIPPTDFDALICGSGSEVYYPSTAQCVDAGGRLRPDQDYLLHINHRWSHDGAKQTIAKLAHDGSGTNVEPDVESCNPHCVSFFIKDPNKVRTIDEMRERVRMRGLRCHLMYCRNATRLQVVPLLASRSQALRYLFVRWGLSVGNMYLIVGEHGDTDHEEMLSGLHKTVIIRGVTEKGSEQLVRSSGSYQREDVVPSESPLIAFTKGDLKADEIMRALKEVTKAASGM

SEQ ID NO: 114 - protein, 716 aa, Oryza sativa
MASVDPSRSFVRDVKRVIIKVGTAVVSRQDGRLALGRVGALCEQVKELNSLGYEVILVTSGAVGVGRQRLRYRKLVNSSFADLQKPQMELDGKACAAVGQSGLMALYDMLFNQLDVSSSQLLVTDSDFENPKFREQLTETVESLLDLKVIPIFNENDAISTRKAPYEDSSGIFWDNDSLAGLLALELKADLLILLSDVDGLYSGPPSEPSSKIIHTYIKEKHQQEITFGDKSRVGRGGMTAKVKAAVLASNSGTPVVITSGFENRSILKVLHGEKIGTLFHKNANLWESSKDVSTREMAVAARDCSRHLQNLSSEERKKILLDVADALEANEDLIRSENEADVAAAQVAGYEKPLVARLTIKPGKIASLAKSIRTLANMEDPINQILKKTEVADDLVLEKTSCPLGVLLIVFESRPDALVQIASLAIRSGNGLLLKGGKEAIRSNTILHKVITDAIPRNVGEKLIGLVTTRDEIADLLKLDDVIDLVIPRGSNKLVSQIKASTKIPVLGHADGICHVYIDKSADMDMAKHIVMDAKIDYPAACNAMETLLVHKDLMKSPGLDDILVALKTEGVNIYGGPIAHKALGFPKAVSFHHEYSSMACTVEFVDDVQSAIDHIHRYGSAHTDCIVTTDDKVAETFLRRVDSAAVFHNASTRFSDGARFGLGAEVGISTGRIHARGPVGVEGLLTTRWILRGRGQVVNGDKDVVYTHKSLPLQ

SEQ ID NO: 115 - DNA, 25 bp, synthetic construct
  misc_feature 1..25: note = Artifical
  source 1..25: mol_type = other DNA
tttaccgggaattgtcgaattaggc

SEQ ID NO: 116 - DNA, 25 bp, synthetic construct
  misc_feature 1..25: note = Artificial
  source 1..25: mol_type = other DNA
tttaaaattcgctggaggaaaaaaa

SEQ ID NO: 117 - DNA, 24 bp, synthetic construct
  misc_feature 1..24: note = Artificial
  source 1..24: mol_type = other DNA
tttgccttggattttccacttgca

SEQ ID NO: 118 - DNA, 25 bp, synthetic construct
  misc_feature 1..25: note = Artificial
  source 1..25: mol_type = other DNA
tttgggggggcttcgctgcctgaat

SEQ ID NO: 119 - DNA, 25 bp, synthetic construct
  misc_feature 1..25: note = Artificial
  source 1..25: mol_type = other DNA
tttgcaagcaagatttcctttgctt

SEQ ID NO: 120 - DNA, 25 bp, synthetic construct
  misc_feature 1..25: note = Artificial
  source 1..25: mol_type = other DNA
tttctcatactcgttggctcgttgc

SEQ ID NO: 121 - DNA, 25 bp, synthetic construct
  misc_feature 1..25: note = Artificial
  source 1..25: mol_type = other DNA
tttatgcggcatctgtctcgtgaac

SEQ ID NO: 122 - DNA, 25 bp, synthetic construct
  misc_feature 1..25: note = Artificial
  source 1..25: mol_type = other DNA
tttaggacctctagaggctacttct

SEQ ID NO: 123 - DNA, 25 bp, synthetic construct
  misc_feature 1..25: note = Artificial
  source 1..25: mol_type = other DNA
tttggtggactactcgatctctgcg

(19) United States
(12) Patent Application Publication
(10) Pub. No.: US 2024/0114848 A1
(43) Pub. Date: Apr. 11, 2024

(54) DEVICES FOR GROWING PLANTS IN THE SEA

(71) Applicant: ALORA INNOVATIONS INC., San Francisco, CA (US)

(72) Inventors: Luke YOUNG, Kitchener (CA); Rory HORNBY, Kitchener (CA); Alex MORRISON, Waterloo (CA)

(21) Appl. No.: 18/045,768

(22) Filed: Oct. 11, 2022

(51) Int. Cl. A01G 9/02; A01G 22/22

(52) U.S. Cl. CPC A01G 9/028; A01G 22/22; A01H 1/1225

(57) ABSTRACT

Disclosed are a plant propagation pod, and an associated support structure, for growing a plant in the sea. The present invention also relates to associated assemblies and methods.

### SEQUENCE LISTING

The instant application contains a Sequence Listing which has been submitted electronically in XML format and is hereby incorporated by reference in its entirety. Said XML copy, created on May 15, 2023, is named 51606-003001_Sequence_Listing_5_15_23 and is 163,192 bytes in size.

### FIELD OF THE INVENTION

The present invention relates generally to a plant propagation pod, and associated support structure, for growing a plant in the sea. The present invention also relates to associated assemblies and methods.

### BACKGROUND

With increasing global population, in combination with climate change, ensuring global food security is more of a challenge than ever. As well as there being limits on arable land that can be used in an environmentally sustainable manner, rising sea levels and an increased frequency of flooding and associated natural disasters have led to an increased rate of salt water contamination in coastal areas. Soil salinity is one of the most severe problems in agriculture aside from drought.

Approximately 20% of the world's cultivated land and nearly half of irrigated land are affected by salinity, which has become a serious threat to agricultural production limiting plant growth and productivity worldwide. Absorption of excessive salt from saline soils inhibits both root and shoot growth, reduces reproductive activity and affects viability of plants. As a result, salinity is one of the major constraints in geographic range of crop cultivation globally, and, where it does not preclude growth of certain crops, nonetheless substantially affects crop productivity. Additionally, salt accumulation as a result of excessive irrigation, improper drainage, or use of reclaimed water places existing agricultural areas at risk, especially as climate change increases irrigation needs in arid/semiarid regions.

The ability to grow plants on water, rather than on land, could go some way to resolving these problems. Many plants, such as rice, which is normally grown in flooded paddy fields, appear well suited to such a solution. However, pressures on freshwater bodies, in terms of cleanliness and area available, limit the ability to cultivate meaningful quantities of plants in this environment.

The oceans and their associated seas cover around 70% of the planet, and the ability to grow meaningful quantities of arable crops in them would be extremely beneficial. However, the salinity of the oceans and other saltwater or brackish bodies of water presents a major challenge to this. None of the major food crops can survive at even close to this level of salinity. Natural genetic variation in food crops provides limited opportunity for enhancement of salinity tolerance, and even relatively saline resistant crops such as rye and barley have threshold salinity values well below that of saline water sources such as seawater. The limited repertoire of naturally saline tolerant plants also limits the applicability of crossbreeding strategies, as the plant species to be crossbred must generally be in the same genus or closely related genera. The use of genetic modification has therefore been proposed as a method of developing arable plants that may be able to grow, and thrive, in a saltwater environment.

However, even if this can be achieved, oceans and seas still prevent major challenged to crop cultivation. Marine structures are continuously exposed to wave action, which can damage and wear them out, while continued exposure to water presents corrosion and biofouling issues that also reduce the lifespan of such structures. Additionally, access to crops for planting, maintenance and harvesting is a greater challenge on water than on land and needs to be made as simple as possible.

Devices and methods of overcoming some of these challenges are therefore desirable.

### SUMMARY OF THE INVENTION

The present invention solves these and other problems by providing a plant propagation pod and associated support structure, along with associated assemblies and methods.

In particular, the present invention provides a plant propagation pod for germinating a seed and structurally supporting a subsequent plant. The pod comprises a three-dimensional mesh configured to structurally support the roots of a plant. The mesh comprises an upper surface, a lower surface and at least one side surface.

The invention further provides a support structure for growing a plant in the sea, comprising at least one pod connection portion configured to connect to a plant propagation pod. The support structure is configured to float and protrude from the surface of water.

The invention further provides an assembly for growing crops in the sea, comprising a plurality of support structures. Each support structure is connected to and in direct contact with at least one other support structure of the plurality of support structures.

The invention further provides a method, comprising floating a support structure or an assembly of support structures on a body of water, wherein the support structure protrudes from the surface of the water.

### BRIEF DESCRIPTION OF THE DRAWINGS

Specific embodiments of the present invention are described in greater detail below in conjunction with the accompanying drawings in which:

FIG. 1 is a perspective view of an exemplary support structure with four connected plant propagation pods.

FIG. 2 is a perspective view of an exemplary plant propagation pod.

FIG. 3 is a plan view of the pod of FIG. 2.

FIG. 4 is a perspective view of the support structure of FIG. 1 with no connected plant propagation pods.

FIG. 5 is a plan view of the support structure of FIG. 4.

FIG. 6 is an exploded view of the support structure of FIG. 4 showing structural components which can be assembled to form the support structure.

FIG. 7 is a perspective view of a central beam which may be used to construct the support structure of FIG. 4.

FIG. 8 is a perspective view of a side segment which may be used to construct the support structure of FIG. 4.

FIG. 9 is a perspective view of an assembly of the support structures of FIG. 4.

FIG. 10 shows the mechanism of Cpf1 (Cas12a), including the location of cut sites and genetic inserts. Inserts contain corresponding sequences to the overhangs plus the desired promoter-based sequence to direct expression of the chosen gene.

FIG. 11 shows callus induction rates of the Rice varieties TH 3-5, Truong Giang, MHC2 and Ha Phat 3 with different concentrations of 2,4-D and BAP. The concentrations in each treatment are summarised in Table 10.

FIG. 12A, FIG. 12B, and FIG. 12C show an analysis of Java long, Se Zic, Agostano, Hunan and Dichroa rice varieties. FIG. 12A shows percentage of seed from the Java long, Se Zic, Agostano, Hunan and Dichroa Rice varieties that germinated. FIG. 12B shows the callus induction rates of the rice varieties with different concentrations of 2,4-D and BAP. The concentrations in each treatment are summarised in Table 10. FIG. 12C shows the shoot development of the rice varieties with the same treatments. The order of the bars from left to right are: Jaca long, Se Zic, Agostano, Hunan and Dichroa repeated, with a space between each set.

FIG. 13 shows callus induction rates of the rice variety Hayayuki with different concentrations of 2,4-D and BAP. The concentrations in each treatment are summarised in Table 11.

FIG. 14A and FIG. 14B show a qRT-PCR analysis of engineered plants according to the invention. FIG. 14A shows the expression of four genes of interest in the leaf, while FIG. 14B shows the expression of four genes of interest in the root. WT denotes a wild type plant. Genotypes 1, 6, 9 and 10 are four different engineered plants according to the invention and the genes of interest are NHX1, SOS1, HKT1 and SODA1. The order of the bars from left to right are: NHX1; SOS1, HKT1 and SODA1 repeated, with a space between each set.

FIG. 15 shows sequences added to the 5′ and 3′ ends of the DNA inserts. The promoter sequences within the 5′ inserts are underlined.

FIG. 16 shows a distribution of hormones throughout the life cycle of a plant. Presence of a particular hormone is indicated by the coloured boxes.

FIG. 17 shows an example of a yield vs salinity (ECe) plot.

FIG. 18A, FIG. 18B, and FIG. 18C show bar graphs showing the gene expression of OsSOS1, OsSOS2, OsAHA3, OsVHA-A, OsNHX1, OsHKT1, OsSODA1 and OsSOD2 in engineered plants according to the invention. The gene expression is relative to both the reference gene, Actin-3, and to the wild-type plants. A positive value indicates an upregulation of the expression, a negative value indicates a down regulation of the gene expression.

FIG. 18A and FIG. 18B show the normalised gene expression grouped by the engineered plants. In FIG. 18B the data have been grouped by biological sample, whereas the data in FIG. 18A have not. FIG. 18C shows the normalised gene expression grouped by the genes of interest. The engineered plants with their corresponding genes of interest are summarized in Table 6.

FIG. 18A and FIG. 18B the order of the bars from left to right are: Plant A—Os-VHA-A, SOS1, SOS2, OsAHA3, HKT1, SODA1, OsSOD2, space, Plant B—NHX1, Os-VHA-A, SOS2, OsAHA3, HKT1, SODA1, OsSOD2, space, Plant C—SOS1, SOS2, OsAHA3, HKT1, SODA1, OsSOD2, space, Plant D—NHX1, Os-VHA-A, SOS2, OsAHA3, HKT1, SODA1, OsSOD2, space, Plant E1—Os-VHA-A, SOS1, SOS2, OsAHA3, HKT1, SODA1, OsSOD2, space, Plant F Os-VHA-A, SOS2, OsAHA3, HKT1, SODA1, OsSOD2, space, Plant G—Os-VHA-A, SOS2, OsAHA3, HKT1, SODA1, OsSOD2, space, Plant I—SOS1, SOS2, OsAHA3, HKT1, SODA1, OsSOD2, space Plant 1—Os-VHA-A, SOS1, SOS2, OsAHA3, HKT1, SODA1, OsSOD2, space, Plant 9—Os-VHA-A, SOS2, OsAHA3, HKT1, SODA1, OsSOD2, space, Wild type—NHX1; OsVHA-A; SOS1; SOD2; OsAHA3; HKT1; SODA1 and OsSOD2. In FIG. 18C the order of the bars from left to right are: Plant A, Plant B, Plant C, Plant D, Plant E1, Plant F, Plant G, Plant I, Plant 1, Plant 9 and Wild type repeated.

FIG. 19A, FIG. 19B, FIG. 19C, FIG. 19D, FIG. 19E, FIG. 19F, FIG. 19G, FIG. 19H, FIG. 19I, and FIG. 19J show scatter plots mapping the normalized gene expression of engineered plants according to the invention against wild type plants (VVT). The settings on the plot include a fold-change threshold of 2.00 with each sample containing two replicates. FIG. 19A is the scatter plot for engineered plant A; FIG. 19B is the scatter plot for engineered plant B; FIG. 19C is the scatter plot for engineered plant C; FIG. 19D is the scatter plot for engineered plant D; FIG. 19E is the scatter plot for engineered plant E1; FIG. 19F is the scatter plot for engineered plant F; FIG. 19G is the scatter plot for engineered plant I; FIG. 19H is the scatter plot for engineered plant G; FIG. 19I is the scatter plot for engineered plant 1; FIG. 19J is the scatter plot for engineered plant 9. The engineered plants with their corresponding genes of interest are summarized in Table 6.

FIG. 20 shows images of wild type (VVT) and engineered rice plants grown in saline media. The engineered plants with their corresponding genes of interest are summarized in Table 6.

FIG. 21A, FIG. 21B, and FIG. 21C show images of wild type (VVT) and engineered calli grown in saline media. The genes of interest in the engineered calli are OsNHX1, OsVHA-A, OsSOS1 & OsSOS2.

FIG. 22 shows a bar graph showing the gene expression of OsSOS1, OsSOS2, OsAHA3, OsVHA-A, OsNHX1, OsHKT1, OsSODA1 and OsSOD2 in engineered calli according to the invention. The gene expression is relative to both the reference gene, Actin-3, and to the wild-type plants. A positive value indicates an upregulation of the expression, a negative value indicates a down regulation of the gene expression. The genes of interest in the engineered calli are OsNHX1, OsVHA-A, OsSOS1 & OsSOS2. The order of the bars from left to right are: SOS1; SOD2; HKT1; NHX1; OsAHA3; OsVHA-A and SODA1 repeated, with a space between each set.

FIG. 23 shows a bar graph showing the gene expression of OsSOS1, OsSOS2, OsAHA3, OsVHA-A, OsNHX1, OsHKT1, OsSODA1 and OsSOD2 in wild-type calli when grown in medium with 0, 2, 4, 6, 8, 10, 12.5 & 15 g/L of sodium chloride. The gene expression is relative to both the reference gene, Actin-3, and to the wild-type plants. A positive value indicates an upregulation of the expression, a negative value indicates a down regulation of the gene expression. The order of the bars from left to right are: HKT1; NHX1; OsAHA3; OsVHA-A; SODA1 SOS1 and SOD2; repeated, with a space between each set.

FIG. 24A, FIG. 24B, FIG. 24C, FIG. 24D, FIG. 24E, FIG. 24F, FIG. 24G, FIG. 24H, FIG. 24I, FIG. 24J, FIG. 24K, FIG. 24L, FIG. 24M, FIG. 24N and FIG. 24O show scatter plots mapping the normalized gene expression of engineered calli according to the invention against wild type plants (VVT). The settings on the plot include a fold-change threshold of 2.00 with each sample containing two replicates. The genes of interest in the engineered calli are OsNHX1, OsVHA-A, OsSOS1 & OsSOS2.

FIG. 25A, FIG. 25B, FIG. 25C, FIG. 25D, and FIG. 25E show images of three engineered rice plants that are producing seeds. The engineered plants comprise the following genes of interest, OsNHX1, OsVHA-A, OsSOS1, OsSOS2, OsAHA3, OsHKT1, OsSODA1, and either OsSODCC1 or OsSOD2, wherein each gene of interest is operatively linked to an enhancer element. FIG. 25A shows a large plant on the left hand side of the image is a engineered plant according to the invention which is a Troung Giang rice variety, while the two plants on the right hand side are engineered plants according to the invention that are from the Hayayuki rice variety; FIG. 25B shows a closer image of the two engineered rice plants of the Hayayuki rice variety; FIG. 25C is an image showing the panicles and seeds of an engineered rice plant of the Hayayuki rice variety; FIG. 25D is an image of the engineered rice plant of the Troung Giang rice variety;

FIG. 25E is an image showing the panicles and seeds of an engineered rice plant of the Troung Giang rice variety.

FIG. 26 shows a bar graph showing the gene expression of OsSOS1, OsSOS2, OsAHA3, OsVHA-A, OsNHX1, OsHKT1, OsSODA1, OsSODCC1 and OsSOD2 in seedlings 13 and 14. Seedlings 13 and 14 were grown from seed harvested from an engineered rice plant of the Hayayuki rice variety. These seedlings were grown from seeds that were obtained from engineered plants according to the invention normalized relative to a wild-type rice plant.

FIG. 27 shows scatter plots mapping the normalized gene expression of seedlings 13 and 14 normalized relative to a wild-type rice plant. The bars represent a four-fold increase in gene expression.

### DETAILED DESCRIPTION

Throughout this specification and the claims which follow, the word “comprise” and variations such as “comprises” and “comprising”, will be understood to mean that the following integer, step, group of integers or group of steps is included but that other integers, steps, groups of integers or groups of steps are not excluded. By contrast, the word “consist” and variations such as “consists” and “consisting of”, will be understood to mean that the following integer, step, group of integers or group of steps is included and that other integers, steps, groups of integers or groups of steps are excluded.

Throughout this specification and the claims which follow, where method steps are described these steps may be carried out in any order, unless it is apparent from the context that one particular method step must be carried out prior to another method step, or unless the chronological order of the method steps is explicitly stated.

The term “removably” will be understood to mean capable of being removed in normal use, and does not encompass physically breaking or causing physical damage to allow the removal to occur. For example, using adhesive to connect one component to a second component and then ripping the first component from the second component while physically breaking the glue does not constitute a removable connection.

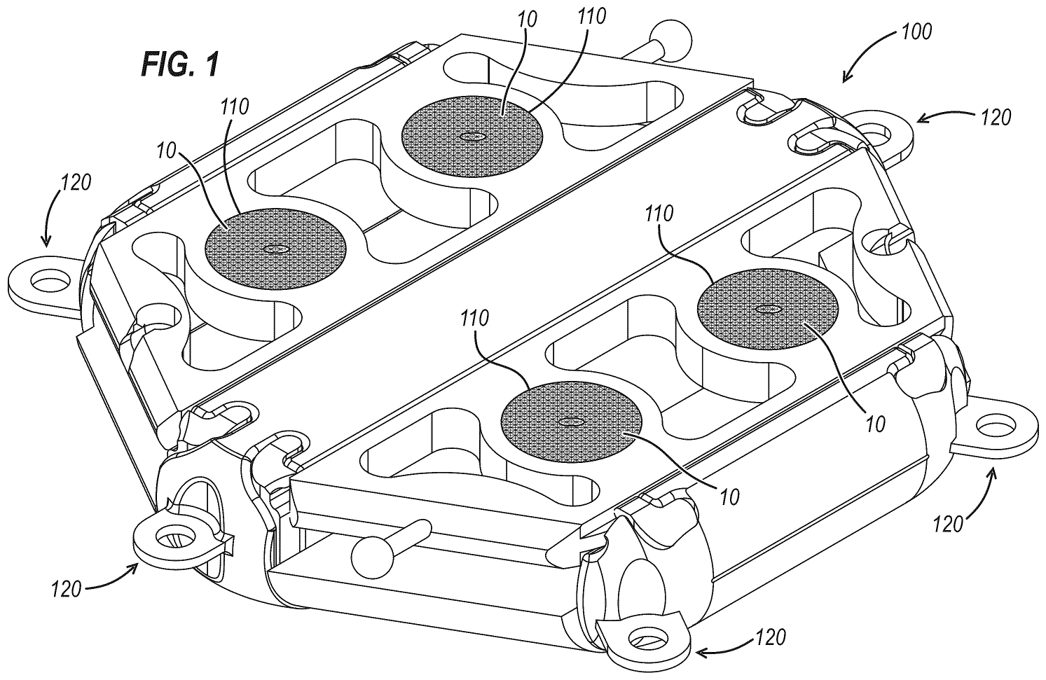

FIG. 1 shows an exemplary support structure 100 for growing a plant in the sea with one or more associated plant propagation pods, such as propagation pod 10. Each plant propagation pod 10 may be connected to a respective pod connection portion 110. In this case, four plant propagation pods 10 are shown, but support structure 100 more comprise more or fewer pod connection portions 110 and associated pods 10. For instance, support structure may comprise one or more, 2 or more, 3 or more, 4 or more 5 or more, 6 or more, 7 or more, 8 or more, 9 or more, 10 or more, 15 or more, 20 or more, 25 or more or 30 or more pod connection portions 110.

Each plant propagation pod 10 may be removably connected to its associated pod connection portion 110, which may allow a plant that is growing in the pod 10 to be germinated, cultivated or harvested elsewhere, away from the structure 100. This allows, for example, a seed to be germinated in pod 10 on land, where conditions may be more favourable given their vulnerability at this stage of life. The plant and pod 10 may be subsequently connected to structure 100 and the plant allowed to grow at sea. Pod 10 may subsequently be removed from structure 100 and taken to land to allow the plant to be harvested. Harvesting pod 10 on land may be easier, and may also facilitate pod 10 being cleaned and re-used.

Alternatively, each pod 10 may be fixedly connected to support structure 100. For example, each pod 10 may be integrally formed with support structure 100, each pod 10 may be an integral part of support structure 100.

Pod

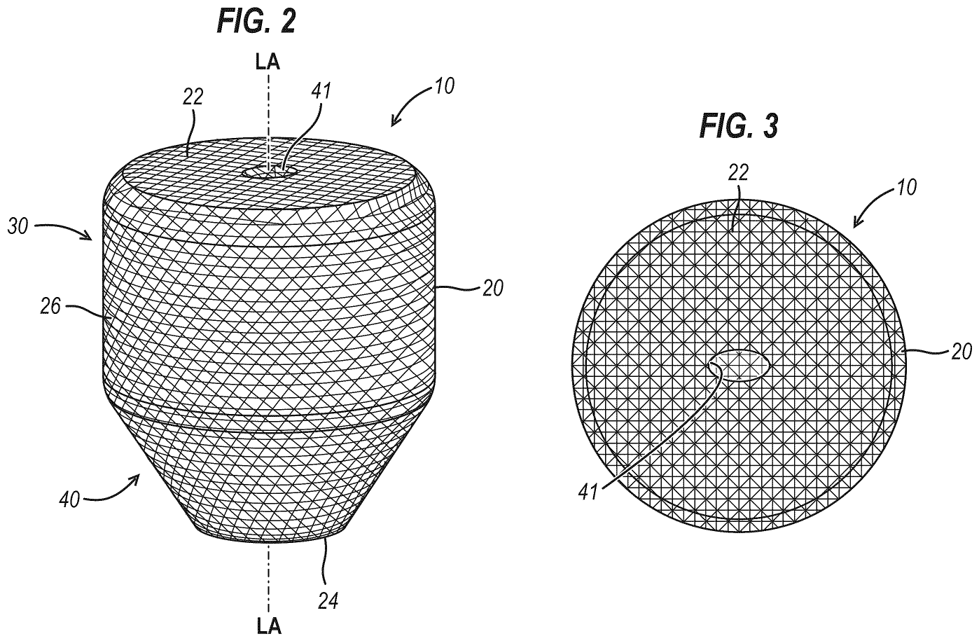

The exemplary plant propagation pod 10 for germinating a seed and structurally supporting a subsequent plant will now be described in greater detail, as shown in FIG. 2. Pod 10, as illustrated, comprises a three-dimensional mesh 20 configured to structurally support the roots of a plant. Mesh 20 comprises an upper surface 22, a lower surface 24 and at least one side surface 26. However, other configurations of pod 10 are possible and some or all of these surfaces may be omitted. In such configurations, reference to the upper, lower and side surfaces below may instead be replaced by reference to an uppermost point or edge, a lowermost point or edge and a side point or edge of the mesh, for example.

Upper surface 22 and lower surface 24 may be planar, and upper surface 22 and lower surface 24 may additionally or alternatively be parallel. Pod 10 may have a longitudinal axis LA-LA that extends through the upper and lower surfaces. Pod 10 may be symmetrical about the longitudinal axis.

Pod 10 comprises a plurality of outer surfaces, and as illustrated by the exemplary pod 10, upper surface 22 and lower surface 24 form one of the plurality of outer surfaces. As illustrated by the exemplary pod 10, at least one side surface 26 also forms one of the plurality of outer surfaces. This arrangement allows the stalk and leaves of the plant to grow and extend above mesh 20 and better access sunlight for photosynthesis, while allowing the roots of the plant to extend below mesh 20 and access the nutrients that are provided by the water in which the plant is growing. Utilising one or more surfaces of mesh 20 as one or more outer surfaces of pod 10 improves air and water flow through the pod by wave or wind action, which improves nutrient flow and keeps pod 10 clear. Furthermore, the quantity of air-water interface within pod 10 is improved, which is beneficial to root and plant growth.

Pod 10 may comprise an upper portion 30 and a lower portion 40 connected to upper portion 30. The at least one side surface 26 may form at least one side wall of upper portion 30. Upper portion 30 may be prismatic. For example, upper portion 30 may be cylindrical, with the at least one side wall 26 forming the side wall of cylindrical upper portion 30. The use of a prismatic upper portion 30 may facilitate the connection of pod 10 with support structure 100. Lower portion 40 may taper towards the longitudinal axis LA-LA of pod 10 as it extends away from upper portion 30. Lower portion 40 may have the shape of a truncated cone or a truncated pyramid. As well as providing further support to the roots of a plant, the shape of lower portion 40 of mesh 20 may assist in connecting pod 10 with pod connection portion 110 of support structure 100.

The size and shape of pod 10 may be configured to match pod connection portion 110 to facilitate connection of pod 10 to pod connection portion 110. As shown in FIG. 2, pod 10 has a circular cross section (i.e upper portion 30 has a cylindrical shape), but other cross-sectional shapes are possible, and may include, for example a square, a rectangle, a triangle or a hexagon.

The mesh is configured to structurally support the roots of a plant, and anchors the plant to the pod to allow it to grow and thrive. In particular, the roots of a plant can embed within the mesh to structurally support the plant. Mesh 20 acts similarly to soil, or another growth medium, in allowing the plant to anchor itself securely while allowing the roots to access nutrients. However, beneficially, as mesh 20 is an integral part of pod 10 it will not be washed away or dispersed by the wave action in the marine environment, and will allow the roots of the plant to be fed by seawater to allow the plant to grow and thrive. In particular, the roots of the plant may extend fully through mesh 20, and through structure 100 when pod 10 is connected to the structure, allowing the ends of the roots of the plant to extend into the sea itself.

As also shown in FIG. 2, mesh 20 occupies an overall mesh volume, and comprises both mesh material and void space between the mesh material. A volume fraction of the volume of mesh material to overall mesh volume can be defined by dividing the volume of mesh material by the overall mesh volume. The volume fraction of mesh material should be sufficiently high that mesh 20 is structurally strong enough to support the roots of a plant, while sufficiently low that the roots of the plant still have room to grow through mesh 20 to allow the roots to anchor to it and reach the water below. To achieve this, the volume fraction of mesh material to total mesh volume is between 10% and 50%, optionally 20% and 40%, further optionally between 25 and 35%. To further assist with this, mesh 20 may additionally or alternatively have a lattice cell with a minimum dimension or maximum dimension of 1 mm or more to 5 mm or less, optionally 2 mm or more to 4 mm or less, further optionally between 2.5 mm or more to 3.5 mm or less. Mesh 20 may have a tetrahedral lattice, a gyroid lattice, a square lattice, an octet lattice, an isotruss lattice, a Voronoi mesh, a fluorite mesh, Delaunay mesh, or a simple cubic lattice,

Mesh 20 may occupy at least 10%, at least 20%, at least 30%, at least 40%, at least 50%, at least 60%, at least 70%, at least 80%, at least 90% or 100% of the volume of pod 10. Pod 10 may consist of mesh 20. A greater mesh volume provided the roots of the plant with more attachment points to anchor to, leading to a more secure plant and reduce the risk that it is damaged. A greater mesh volume also increases the volume of water and air that is retained by the mesh, and the quantity of air water interface within pod 10. Air-water interface is beneficial to root and plant growth, and so this further benefits the plant.

The height of mesh 20 is sufficient to allow the roots of the plant to securely embed within the pod such that the plant is structurally supported. If the height of the mesh is too low, then it is harder for the roots of the plant to embed deeply enough to resist the turning moment generated by wind, gravity, and/or wave action which would tend to cause the plant to fall over. Mesh 20 may therefore comprise a plurality of lattice cells, or layers of lattice cells, along longitudinal axis LA-LA or height of pod 10. Stated another way, longitudinal axis LA-LA extends through a plurality of lattice cells as it extends between upper surface 22 and lower surface 24. Stated another way, pod 10 comprises a plurality of vertically stacked lattice cells along the height direction of pod 10. Longitudinal axis LA-LA may extend through at least 2, at least 3, at least 4, at least 5, at least 6, at least 7, at least 8, at least 9, at least 10, at least 15, at least 20, at least 25, at least 30, at least 40 or at least 50 lattice cells of pod 10 as it extends between upper surface 22 and lower surface 24. Stated another way, pod 10 may comprise at least 2, at least 3, at least 4, at least 5, at least 6, at least 7, at least 8, at least 9, at least 10, at least 15, at least 20, at least 25, at least 30, at least 40 or at least 50 vertically stacked lattice cells along the height direction of pod 10.

Additionally or alternatively, the width or length of pod 10 divided by the height of pod 10 may be 0.1 or more to 10 or less, optionally 0.2 or more to 5 or less, optionally 0.25 or more to 4 or less, further optionally 0.33 or more to 3 or less, further optionally 0.5 or more to 2 or less, further optionally 0.66 or more to 1.5 or less. Similarly, this may ensure that the vertical dimension of pod 10 is sufficiently high that it can support the plant. The height of pod 10 may be 2 cm or more and 10 cm or less, optionally 3 cm or more and 8 cm or less, further optionally 4 cm or more and 6 cm or less. The width or length of pod 10 may be 2 cm or more and 20 cm or less, optionally 2.5 cm or more to 10 cm or less, optionally 3 cm or more and 8 cm or less, further optionally 4 cm or more and 6 cm or less. Different pod heights, lengths and widths can be selected depending on the type of plant that is growing in the pod.

A hole 41 may be defined in mesh 20, and may as illustrated be defined in upper surface 22 of mesh 20. Hole 41 may be a recess. Hole 41 may be in the centre of upper surface 22, and longitudinal axis LA-LA may pass through hole 41. Hole 41 may extend a distance of a plurality of lattice cells into mesh 20. However, hole 41 may extend no more than 5%, no more than 10%, no more than 15%, no more than 20%, no more than 25% or no more than 30% of the distance from upper surface 22 to lower surface 24. Hole 41 provides a space for a seed to germinate inside pod 10, and is in proximity to upper surface 22 of pod 10 so that the hypocotyl and cotyledons of the resulting seedling can quickly move past upper surface 22 to enable improved photosynthesis, while the roots of the seedling can still embed firmly in mesh 20.

In alternative embodiments, pod 10 may comprise a wall (not shown). The wall may have an outer surface and an inner surface. The inner surface may define an internal space. Mesh 20 may occupy at least a portion of the internal space and may be connected to the inner surface of the wall. Mesh 20 may extend from the inner wall, and may extend from and be in physical contact with inner wall around a full perimeter of the inner surface of the wall. Mesh 20 may be integrally formed with the inner surface of the wall.

Pod 10 may be inflexible (i.e. not flexible) or rigid, to protect it from deforming in the harsh marine environment and improve the support that it provides to a plant which is propagated in it. In other words, pod 10 may not be deformable in use. If pod 10 does deform, it may resiliently deform under loading, or may resiliently deform when initially loaded. In other words, if pod 10 is deformed under loading, pod 10 will return to the shape that it had prior to the loading once the loading is stopped. Pod 10 may be inflexible, rigid, or resiliently deform under loading even if it is not connected to another structure, such as structure 100. This, again, improves the ability of pod 10 to support the plant and its roots and ensure that the plant is protected. In addition or alternatively to this, mesh 20 may be inflexible or rigid, or may resiliently deform under loading.

Pod 10 may comprise a synthetic material. Synthetic materials tend to be more corrosion resistant and resistant to organic degradation or biofouling than natural materials. In particular, pod 10 may comprise plastic, such as HDPE, ABS, biodegradable plastic, and/or plant derived plastic. The plastic may be rigid or may resiliently deform under loading. Plastic is particularly resistant to the corrosive effects, such as rusting, of a marine environment. Certain plastics, such as HDPE, have low rates of water absorbance which assists in maintaining their structural performance even after long duration exposure to water, salt water or sea water. Additionally or alternatively, mesh 20 may comprise or consist of a synthetic material or a plastic, such as HDPE, ABS, biodegradable plastic, and/or plant derived plastic. The plastic may be rigid or may resiliently deform under loading.

Pod 10 may comprise connection means (not shown) to allow pod 10 to connect to a floating structure, such as structure 100 and the pod connection portions 110 or structure 100. The connection means may comprise a screw thread, a ridge or a protrusion. For example, a screw thread may be applied around at side surface 26 of pod 10. Additionally or alternatively, a ridge or protrusion may be applied to side surface 26 of pod 10. The ridge or protrusion may form an interference fit with pod connection portion 110 (which may be a pod receiving hole as explained below) when pod 10 is connected to pod connection portion 110.

Pod 10 may be manufactured by molding, such as injection molding. Alternatively, pod 10 may be 3D printed by powder bed fusion or resin printed. Alternatively pod 10 may be foamed.

### Support Structure

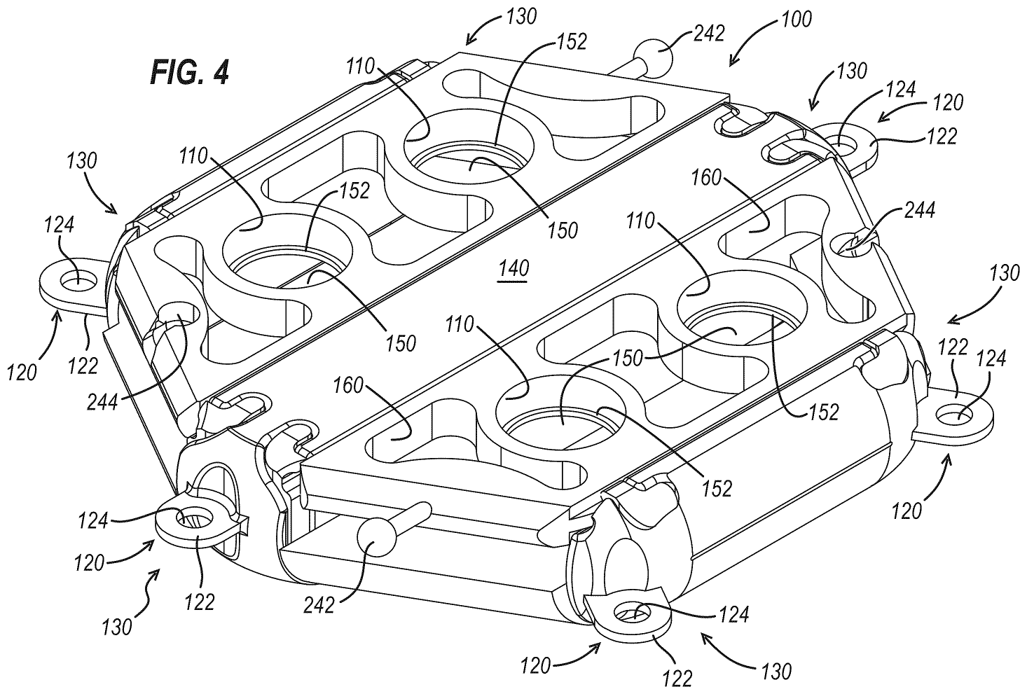

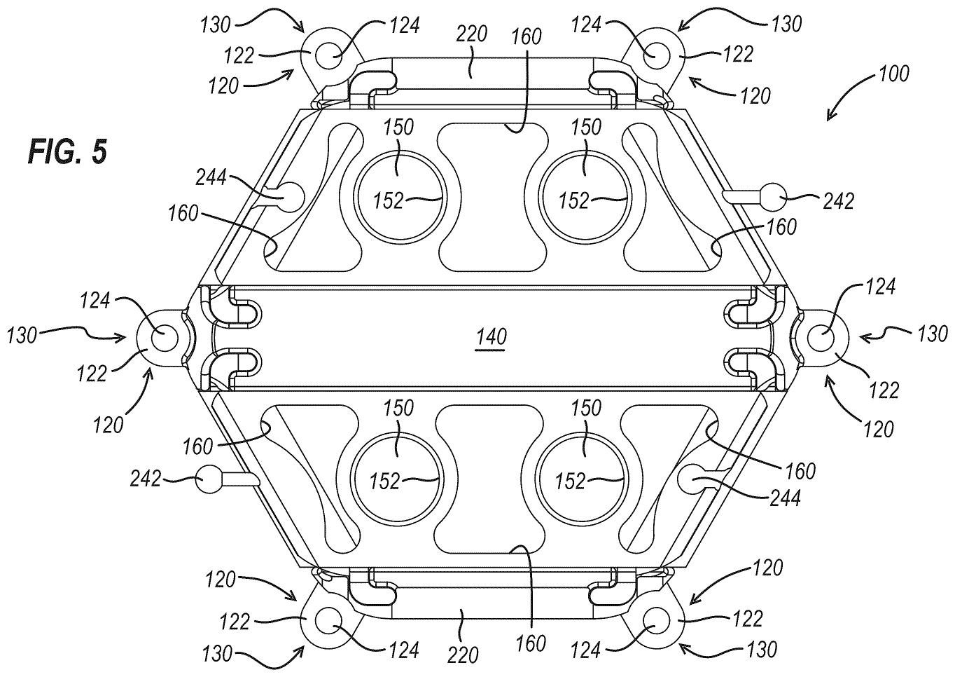

Further views of support structure 100, without any pods 10 connected to any of the pod connection portions 10, are shown in FIG. 4 and FIG. 5. As with pod 10, support structure 100 may comprise a synthetic material, due to their resistance to corrosion, organic degradation and biofouling. In particular, support structure 100 may comprise plastic, such as HDPE, ABS, biodegradable plastic, and/or plant derived plastic. The plastic may be rigid or may resiliently deform under loading. Support structure 100 may furthermore consist essentially of or consist of such materials. At least one component of support structure 100 may be hollow, and may comprise rigid, inflexible or resiliently deformable walls enclosing the internal hollow space. Using at least one hollow component improves the buoyancy of the support structure and allows it to support heavier plants with a smaller volume as a result, while also being straightforward to manufacture using manufacturing techniques such as rotation or blow molding.

Support structure 100 comprises at least one structure connection portion 120, which is configured to enable support structure 100 to be connected to a second support structure, which may also be a support structure 100. The at least one structure connection portion 120 allows an assembly of structures to be created, which allows a modular farm of connected structures which can flex relative to each other to be created, and in which plants can be grown in propagation pods 10. Such a modular assembly is advantageous, as it allows the assembly to adapt to varying water levels caused by wave action across the full extent of the assembly. Plant development is optimized when the roots are submerged without having the rest of the plant submerged, and therefore it is beneficial to maintain a specific water level as best as possible. When an assembly is rigid, the water level varies greatly as the structure increases in lateral surface area, therefore limiting potential size of the assembly. Consequently, having an assembly of support structures that is flexible is desirable.

In particular, support structure 100 may comprise a plurality of corners 130, and a support structure connection portion 120 may be provided at each corner 130. Alternatively, a support structure connection portion 120 may be provided between each pair of corners 130. Stated another way, each corner 130 may form two pairs of corners (one pair with each adjacent corner) around the perimeter of support structure 100, and there may be a support structure connection portion 120 located between each such pair of corners.

The at least one support structure connection portion 120 may comprise a flange 122 which may comprise a hole 124. Hole 124 may receive a pin or an alternative fastener, which may allow support structure 100 to be removable or permanently connected to another support structure 100. Alternative forms of connection portion are, however, possible.

Support structure 100 may comprise a top surface 140. When support structure 100 floats on a stationary fluid, top surface 140 may be parallel to the surface of the fluid. Top surface 140 may comprise the at least one pod connection portion 110. Specifically, the at least one pod connection portion 110 may be a pod receiving hole 150, and each pod connection portion 110 may be a pod receiving hole 150. Each pod receiving hole 150 extends through the top surface and is a through hole. Each pod receiving hole 150 is configured to receive a pod 10. The perimeter of one or more pod receiving holes 150 may be configured to match or substantially match an outer perimeter of pod 10, so as to securely receive pod 10. The perimeter of one or more pod receiving holes 150 may comprise a key or cut-out portion (not shown) that does not match the outer perimeter of pod 10, so as to allow the inserted pod 10 to be gripped and removed from pod receiving hole 150.

Each pod receiving hole 150 may comprise a ledge 152. Ledge 152 is configured to support the pod 10 that may be received in hole 150, so that it remains seated in hole 150. Upper surface 22 of each pod 10 may be level with top surface 140 when a respective pod is received in its respective pod receiving hole 150. Mesh 20 of each pod 10 may protrude beyond pod receiving hole 150 in the direction away from top surface 140. In other words, mesh 20 of each pod 10 may protrude below its respective hole 150, which may improve access to water for the roots of the plant support by the pod by supporting them further into the water on which support structure 100 is floating.

Support structure 100 may further comprise one or more holes 160 that pass fully through the support structure. One or more holes 160 may pass through top surface 140. The combined area of the plurality of holes may be at least 5%, at least 10%, at least 15%, at least 20%, at least 25%, at least 30%, at least 35%, at least 40%, at least 45%, at least 50%, at least 55% or at least 60% of the area enclosed by the perimeter of the support structure. One or more holes 160 may reduce the amount of light pollution generated by structure 100. In other words, holes 160 ensure that support structure 100 blocks less light, which is beneficial for marine life that may grow below support structure 100.

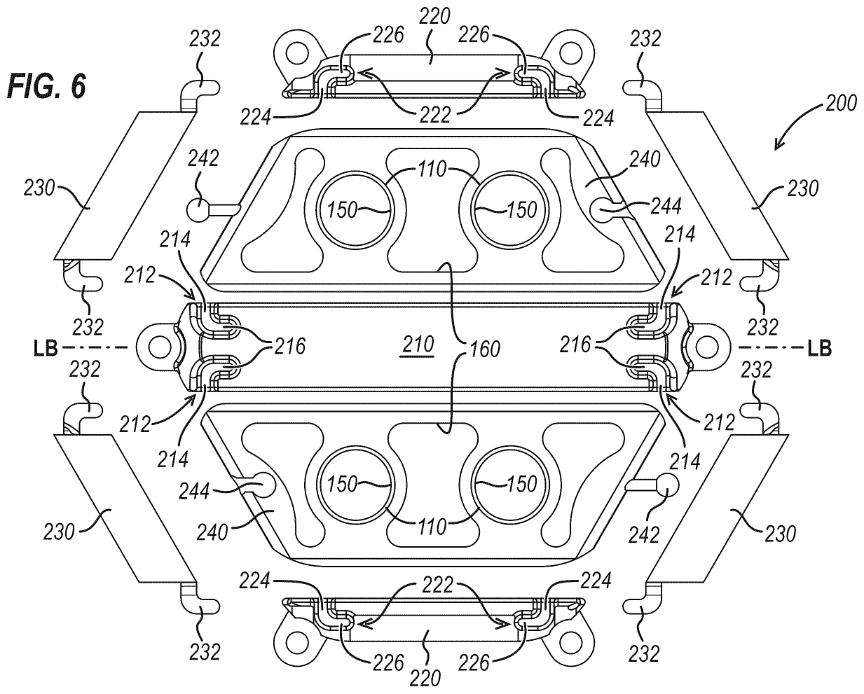

FIG. 6 shows an exploded view of a plurality of structural components 200 that can be assembled to form support structure 100. More specifically, it may be possible to securely assemble the plurality of structural components 200 to form support structure 100 without fasteners. In other words, it may be possible to assemble the plurality of structural components 200 to form support structure 100, such that support structure 100 is structurally integral and will not fall apart in use, without the use of fasteners. One or more, and possibly all, of the components of the plurality of structural components may be hollow. The components of the plurality of structural components 200 can be slotted together to securely form the support structure 100. In other arrangements, however, fasteners may be used.

As illustrated in FIG. 6, the plurality of structural components 200 may comprise nine components, which may comprise centre beam 210, two side segments 220, four brace segments 230 and two trays 240, although other arrangements are possible. In particular, in some arrangements, which are not illustrated, support structure 100, or the body of support structure 100, may be made from one single integral component.

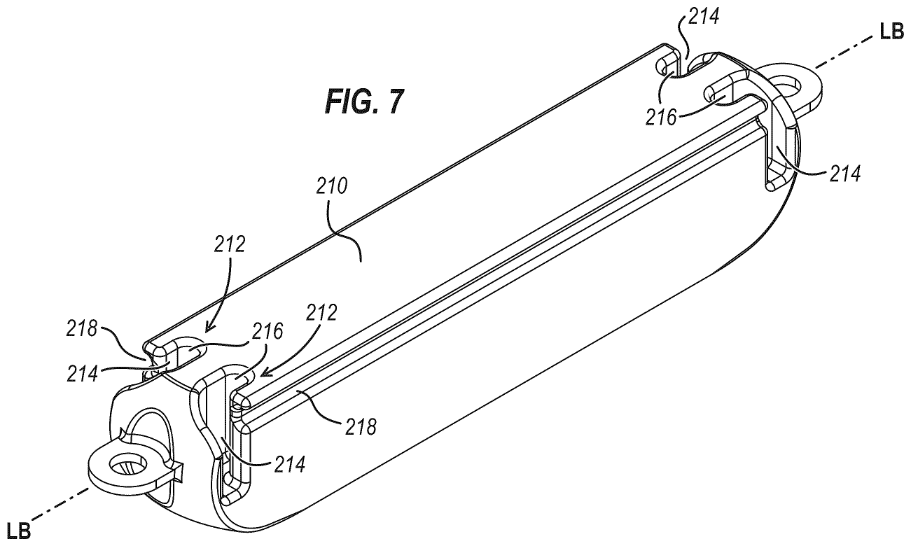

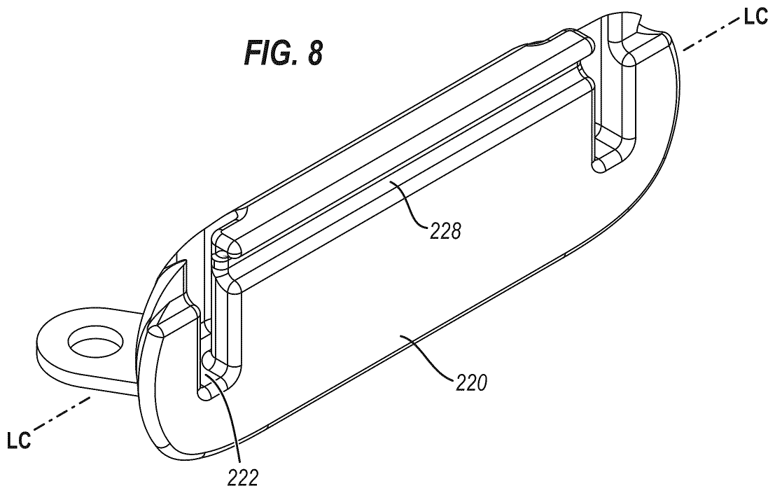

As shown by FIG. 6 and FIG. 7, centre beam 210 comprises four brace slots 212 and two tray slots 218 (one not shown). Each brace slot 212 may comprise a slot entrance 214 and a slot end 216, with slot end 216 projecting in a different direction to slot entrance 214. In other words, slot end 216 is angled with respect to slot entrance 214. Centre beam 210 may define a longitudinal axis LB-LB, and the slot entrance 214 of each brace slot 212 may project in a different direction to axis LB-LB. Additionally, each tray slot 218 may extend parallel to longitudinal axis LB-LB. As shown by FIG. 6 and FIG. 8, each side portion 220 comprises comprises two brace slots 222 and one tray slots 228. Each brace slot 222 may comprise a slot entrance 224 and a slot end 226, with slot end 226 projecting in a different direction to slot entrance 224. In other words, slot end 226 is angled with respect to slot entrance 224. Each side portion 220 may define a longitudinal axis LC-LC, and the slot entrance 224 of each brace slot 222 may project in a different direction to axis LC-LC. Additionally, each tray slot 228 may extend parallel to longitudinal axis LC-LC.

As shown in FIG. 6, each brace segment 230 may comprise one or more protrusions 232 configured to slidably connect with brace slots 212 and 222, with protrusion 232 configured to simultaneously fit into both of a respective slot entrance 214 or 224 and a respective slot end 216 or 226. Due to the angle between a respective slot entrance 214 or 224 and respective slot end 216 or 226, protrusion 232 cannot slide out of a respective slot 212 or 222 along the axis of the slot, and each brace segment 230 must therefore be lifted out of a connected side segment 220 or centre beam 210 to disconnect these components. The protrusions 232 may slidably connect with slots 212 and 222 with an interference fit, but other arrangements are possible.

As shown by FIG. 6, FIG. 7, and FIG. 8, each tray 240 slidably connects with its respective tray slots 218 and 228. As each tray slot 218 and 228 extends in a different direction to the direction in which brace segments 230 can be disconnected from centre beam 210 and side segments 220, the insertion of a tray 240 prevents the disassembly of respective brace segments 230 from the centre beam 210 and its respective side segment 220. Additionally, as shown, each tray slot 218 or 228 may extend across slot opening 214 or 224, which may further block respective protrusions 232 of the brace segments 230 from being removed from their respective slots 212 and 222. Each tray 240 therefore holds or locks the plurality of structural components 200 together.

While the above assembly arrangement has been described with centre beam 210, two side segments 220, four brace segments 230, other arrangements are possible. For example, centre beam 210, two brace segments 230 and one tray 240 can be omitted, such that each side segment 220 connects to the two remaining brace segments 230 in a quadrilateral arrangement, with the tray 240 holding these together. Additionally or alternatively, centre beam 210 and/or side segments 220 may comprise the protrusions, with respective slots formed instead on brace segments 230.

As also shown by FIG. 6, each tray 240 may comprise one or more of the pod connection portions 110. As tray is easily removable from support structure 100, this may facilitate the easy removal or insertion of connected pods 10, simplifying the addition of previously germinated plants to the structure 100 and the removal of these plants for maintenance, feeding or harvesting. As such, each tray 240 may have a loose fit with tray slots 218 and 228, rather than an interference fit, to facilitate easy insertion and removal of each tray 240. Tray 240 may also comprise tray male connection 242 and tray female connection 244. This allows tray 240 to connect direction to a tray of an adjacent support structure 100 as explained in more detail below.

Support structure 100 may have the shape of a square, a triangle, an equilateral triangle, a hexagon, a regularly hexagon or a rectangle. In particular, support structure may have a shape that allows a number of support structures of the same shape to tessellate. This advantageously allows assemblies of support structures to be created without gaps, as describes in greater detail below.

Support structure 100 may have a maximum dimension parallel to top surface 140 of 10 cm or more and 150 cm or less, optionally 20 cm or more and 100 cm or less, optionally 25 cm or more and 50 cm or less, optionally 30 cm or more and 40 cm or less. Advantageously, dimensions such as these are comparable to regular wave sizes in the sea and other bodies of water. These dimensions therefore allow support structure 100 to be as large as possible, maximising plant growth, while preventing structure 100 regularly straddling more than one wave, causing support structure 100 and its plants to be buffeted and possible damaged.

For a support structure 100 comprising 2 or more pod connection portions 110 (such as pod connection holes 150), pod connection portions 110 may be arranged such that there is a minimum distance between a pod connection portion 110 and a second pod connection portion 110. This arrangement may ensure that the plant of each pod 10 has sufficient room to grow, and does not have to compete with neighbouring plants and reduce overall yield. In particular, pod connection portions 110 may be arranged so that each pod connection portion 110 is no closer than the minimum distance to any other pod connection portion 110. The minimum pod connection portion may be 0 cm or more and 30 cm or less, optionally 5 cm or more and 25 cm or less, optionally 10 cm or more and 20 cm or less, optionally 12.5 cm or more and 17.5 cm or less.

Support structure 100 and/or components of the plurality of structural components 200 may be manufactured by molding, such as injection molding, rotation molding or blow molding. Alternatively, support structure 100 and/or components of the plurality of structural components 200 may be 3D printed by powder bed fusion or resin printed.

The present invention also relates to a kit of parts for support structure 100, and a method of assembly support structure 100, which may avoid the use of fasteners as discussed above, for example.

Assembly

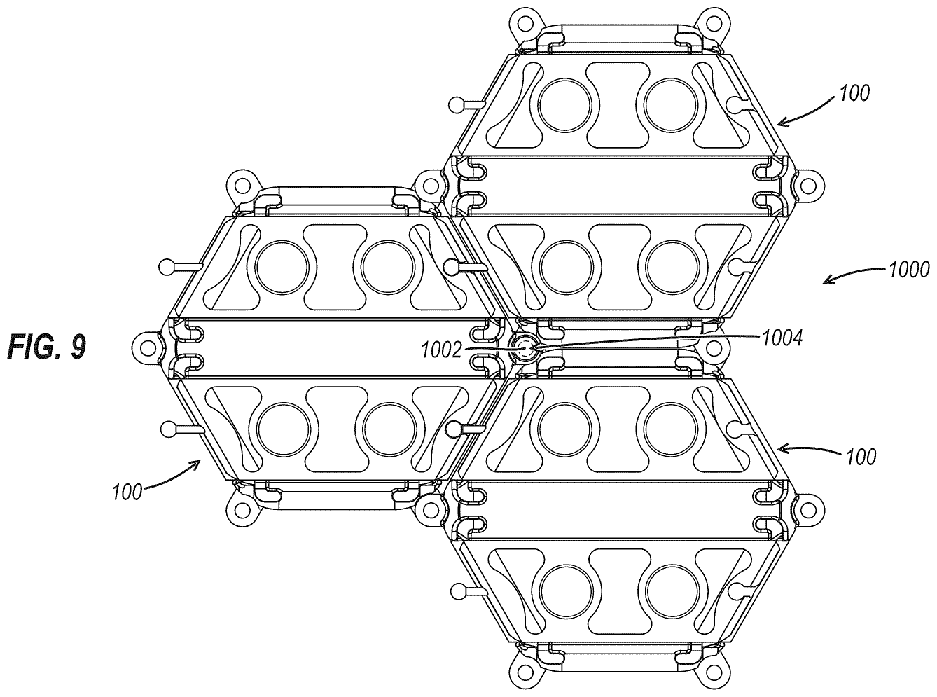

FIG. 9 depicts an exemplary assembly of structures 1000. The assembly comprises two or more of support structure 100 connected to and in direct contact with at least one other support structure 100 of the assembly 1000. FIG. 9 shows three of support structure 100 connected together, with a pin 1002 connecting the holes 124 in a flange 122 of a structure connection portion 120 of each of the three structures 100 together. A cap (not shown) may be permanently or removably connected to pin 1002, and pin 1002 may comprise a screw and the cap may comprise a nut. More generally, assembly 100 may comprise three or more support structures 100, and at least three of the support structures 100 in assembly 1000 may be connected to and in direct contact with the other two of the three support structures 100. The support structures 100 may be permanently or removably connected together. As shown in FIG. 9, such an arrangement of support structures 100 may tessellate together. Each structure 100 in the assembly may tessellate together, and each such support structure 100 may have the same shape. Further pins 1002 may be provided at other structure connection portions 120 of the structures 100 to further connect the structures 100 together.

The connections of the structures 100 together via the structure connection portions 120 may permit a support structure 100 to flex relative to a neighbouring structure 100 in the assembly 1000. Specifically, the connections in assembly 1000 may allow a support structure 100 to angularly flex relative to an adjacent structure, which allows the assembly 100 to advantageously flex with undulating surface of a body of water caused by wave action. Additionally, the connection in assembly 1000 may allow a support structure 100 to simultaneously flex relative to each of its adjacent structures 100, even if it is in contact with more than one adjacent structure. This may be achieved, for example, by ensuring that pin 1002 is relatively loose in each slot 124 of the respective structure connection portions 120 of the structures 100. Alternatively, pin 1002 may have a tight fit or an interference fit in each slot 124, with flex between adjacent support structures 100 being provided for by flexing or resiliently deformation of one or more of the support structures 100. Each of the three support structures 100 may be capable, for example. of flexing at an angle of 5 degrees or more and 20 degrees or less relative to the other two support structures 100 of the three support structures 100, optionally an angle of 7.5 degrees or more and 10 degrees or less, which may allow the assembly 1000 to flex and conform to common curvatures of waves on the sea and other bodies of water.

As shown in FIG. 9, the given support structures 100 of an assembly 1000 may comprise a central vertex, and each support structure may comprise a structure connection portion 120 at the central vertex 1004. The connection portions 120 at the central vertex 1004 may all be joined together, such as by pin 1002.

Each support structure 100 may comprise at least two vertices, such as corners 130, with each support structure 100 in which it is in contact, with each support structure 100 comprising a connection portion 120 at each of the at least two vertices. The connection portions 120 of the adjacent structures 100 at the respective at least two vertices may be joined together as described above, for example by a pin 1002.

Larger assemblies 1000 of support structures 100 may be created. For instance, assemblies of 5, 10, 20, 30, 50, 100 or more support structures 100 may be created by connecting a plurality of support structures 100 together. Each such assembly 1000 may comprise at least one arrangement of three support structures as shown in FIG. 9, for example.

As shown in FIG. 9, a tray 240 of a support structure 100 may be connected to an adjacent tray 240 of an adjacent support structure 100 through a tray male connection 242 and a tray female connection 244. As tray slots 218 and 228 of the two adjacent support structure 100 align as shown, it is possible for multiple trays to be inserted or removed in a single movement, further simplifying the addition of previously germinated plants to the structure 100 and the removal of these plants of maintenance, feeding or harvesting.

The present invention further relates to a kit of parts for an assembly 1000, and a method of constructing an assembly 1000 which comprises connecting a plurality of support structures 100 together such that each support structure 100 is connected to and in direct contact with at least one other support structure 100 of the plurality of support structures 100.

Method of Growing a Plant

The present invention also relates to a method comprising floating a support structure 100 or an assembly of support structures 1000 on a body of water, wherein the support structure protrudes from the surface of the water. The body of water may be salt water, such as the sea or the ocean. The salinity of the body of water may be greater than 2, 5, 8, 12, 18, 25 30 or 35 parts per thousand.

The method may further comprise growing at least one plant in at least one pod 10 of the support structure 100 or assembly 1000. The method may comprise growing a plurality of plants in pods 10 of the support structure 100 or assembly 1000, or a plant in each of the pods 10 of the support structure 100 or assembly 1000.

The method may further comprise germinating at least one plant in the at least one pod 10 or the pods 10, such as in the hole 41 of the respective pods 10. Germinating the plant in pod 10 may comprise germinating the plant in the at least pod on land, prior to floating structure 100 or assembly 1000 on the body of water. The method may further comprise connecting the pod 10 to a respective pod connection portion 110 after germination has occurred, optionally after the floating of the support structure 100 or assembly 1000.

The method may further comprise harvesting the plant. Harvesting the plant may comprise removing the at least one plant from support structure 100 or assembly 1000 while the support structure 100 or assembly 1000 continues to float on the body of water, such as by removing a tray 240 with a pod 10 connected to the pod connection portion 110 of the tray 240. Harvesting the plant may alternatively comprise removing support structure 100 or assembly 1000 from the body of water, and removing the at least one plant from support structure 100 or assembly 1000 while support structure 100 or assembly 1000 is not in the body of water. Removing the at least one plant from support structure 100 may comprise removing the respective pod 10 from the respective pod connection portion 110.

Plants

As noted above, pod 10, support structure 100 and assembly 1000 may be used to grow plants in the sea or another body of water. Pod 10, support structure 100 and assembly 1000 may therefore comprise one or more plants, with the roots of each plant embedded within a mesh 20 of a pod 10 so that pod 10 into which the roots are embedded can structurally support the plant. Each pod 10 in a support structure 100 or assembly 1000 may comprise a plant, and each pod may comprise a single plant (i.e. one plant per pod).

The plant may be a genetically engineering salt tolerant plant. In combination with pod 10, structure 100 and assembly 1000, this advantageously allows crops that are normally grown on land, such as rice or corn, to be grown in the sea. Engineering such a plant effectively creates limitless water with which to irrigate crops, solve problems of lack of arable land and salt pollution of such land.

The plants may be transgenic plants and/or may include genes of interest operatively linked to an enhancer element. The transgenes, and/or genes of interest operatively linked to an enhancer element, result in improved salt tolerance by affecting multiple mechanisms as disclosed herein.

An engineered plant may comprising at least two genes of interest, wherein the genes of interest comprise a gene that encodes a protein that controls the intracellular ion concentration and a gene that encodes an antioxidant, wherein the gene that encodes a protein that controls the intracellular ion concentration is operatively linked to an enhancer element and the gene that encodes an antioxidant is operatively linked to an enhancer element.

An engineered plant may comprise at least two genes of interest, wherein the genes of interest comprise a gene that encodes a plasma membrane protein that controls the intracellular ion concentration and a gene that encodes a tonoplast protein that controls the intracellular ion concentration, wherein the gene that encodes a plasma membrane protein that controls the intracellular ion concentration is operatively linked to an enhancer element and the a gene that encodes a tonoplast protein that controls the intracellular ion concentration is operatively linked to an enhancer element

An engineered plant may comprise at least three genes of interest, wherein each gene of interest is operatively linked to an enhancer element, wherein the engineered plant has increased salt tolerance compared to a plant of a same species without said genome modifications.

An engineered plant may comprise at least three genes of interest wherein the genes of interest comprise a gene that encodes a plasma membrane protein that controls the intracellular ion concentration, a gene that encodes a tonoplast protein that controls the intracellular ion concentration and a gene that encodes an antioxidant, and wherein the gene that encodes a plasma membrane protein that controls the intracellular ion concentration is operatively linked to an enhancer element, the gene that encodes a tonoplast protein that controls the intracellular ion concentration is operatively linked to an enhancer element and the gene that encodes an antioxidant is operatively linked to an enhancer element.

An engineered rice plant may comprise at least eight genes of interest, wherein the at least eight genes of interest encode OsSOS1, OsSOS2, OsAHA3, OsVHA A, OsNHX1, OsHKT1, OsSODA1 and OsSOD2 wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; and wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.

An engineered rice plant may comprise at least eight genes of interest wherein the at least eight genes of interest encode OsSOS1, OsSOS2, OsAHA3, OsVHA A, OsNHX1, OsHKT1, OsSODA1 and OsSODCC1;
wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSODCC1 gene comprises a polynucleotide sequence that encodes a polypeptide comprising the amino acid sequence of SEQ ID NO: 94; and wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.

An engineered rice plant may comprise at least eight genes of interest, wherein the eight genes of interest encode OsSOS1, OsSOS2, OsAHA3, OsVHA A, OsNHX1, OsHKT1, OsSODA1 and OsSOD2; wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; wherein the OsSOS1 gene is operably linked to an enhancer element comprising SEQ ID NO: 12, the OsSOS2 gene is operably linked to an enhancer element comprising SEQ ID NO: 13, the OsAHA3 gene is operably linked to an enhancer element comprising SEQ ID NO: 14, the OsVHA A gene is operably linked to an enhancer element comprising SEQ ID NO: 11, the OsNHX1 gene is operably linked to an enhancer element comprising SEQ ID NO: 10, the OsHKT1 gene is operably linked to an enhancer element comprising SEQ ID NO: 15, the OsSODA1 gene is operably linked to an enhancer element comprising SEQ ID NO: 16 and the OsSOD2 gene is operably linked to an enhancer element comprising SEQ ID NO: 18. Also provided is such an engineered rice plant where the sequences defined in this paragraph are varied, e.g. such that any or all of the sequences specified have at least 95% sequence identity to the specified SEQ ID NO.

“Salinity,” as used herein, generally refers to a measure of soluble salts in soil or water. “Salt,” as used herein, generally refers to any molecule comprised of a cation, such as sodium (Na+), potassium (K+), magnesium (Mg2+), or calcium (Ca2+), and an anion, such as chloride (Cl−), bicarbonate (HCO3−), carbonate (CO32−), or sulfate (SO42−). Sodium chloride (NaCl) is the most common salt in groundwater and soils. The salinity of soil (or another growth medium) can be expressed: (a) as the salt concentration of the medium in terms of grams per liter (g/L) or parts per thousand (ppt) or (b) in terms of electric conductivity (EC, in deciSiemens per meter—dS/m, or in equivalent units milliMhos per centimeter—mmhos/cm or milliSiemens per centimeter—mS/cm). For soil, salinity can be measured in units of electrical conductivity of a saturated soil paste extract (ECe) taken from the root zone of a plant and averaged over time and depth. Soil paste extracts are soil samples that are brought up to their water saturation points (see, e.g., USDA. Diagnosis and improvement of saline and alkali soils. Agriculture Handbook No. 60. (1954), which is incorporated by reference herein). In some embodiments, electrical conductivities are measured on the vacuum-extracted and filtered water extracts from saturated soil paste extracts.

According to the USDA salinity laboratory, “saline” can be defined as a medium having an electrical conductivity of the saturated paste extract (ECe) of 4 dS/m or greater, “slightly saline” (or medium) can be defined as having an electrical conductivity of the saturated paste extract (ECe) of between 4 and 8, moderately saline can be defined as having an electrical conductivity of the saturated paste extract (ECe) of between 8 and 16, and severely saline can be defined as having an electrical conductivity of the saturated paste extract (ECe) of greater than 16; and seawater may have a salt concentration of 30 g/L and an EC of 50 dS/m. However, effects can occur on plants at salinity levels lower than 4 dS/m; so-called sensitive plants can exhibit growth problems at 0.75-1.5 dS/m and many plants can nonetheless experience growth rate decreases at 1.5-3.0 dS/m.

As used herein, the term “operably linked” generally means that a promoter sequence or enhancer sequence is positioned with respect to a transcribable or translatable polynucleotide sequence (i.e. the transgene or gene of interest) such that the regulatory element within the promoter sequence can affect the regulatory activity of the polynucleotide sequence. A promoter having transcriptional promoter activity, for example, can be located at any distance, including adjacent to or up to thousands of nucleotides away from, and upstream or downstream from the gene, which can be a minimal promoter element, and polynucleotide sequence to be transcribed, and still exert a detectable effect on the level of expression of an encoded reporter molecule.

An enhancer element is a polynucleotide sequence that comprises or consists of a regulatory element(s) that is/are capable of altering the expression level of a gene of interest compared to the expression level of the gene of interest in a wild-type plant. For example, an enhancer element may increase the expression level of a gene of interest compared to a wild-type plant. The enhancer element may alter the expression level of the gene of interest at all times or at one or more stages of the plant's life cycle, for example when the plant is germinating. The enhancer element may result in the expression of the gene of interest at a different location in the plant compared to a wild-type plant.

The enhancer element can comprise a regulatory element(s) that is/are naturally present in the plant genome or can contain regulatory elements that are not naturally present in the plant genome. For example, the enhancer element may comprise a polynucleotide sequence obtained from or derived from a plant cell of the same species, or another plant cell of a different species, or a non-plant source, or a synthetic sequence. In certain embodiments, the enhancer element is not the promoter sequence that is naturally present in the plant genome for the corresponding gene of interest.

An enhancer element is incorporated into the genome of a plant by plant transformation methods that involve genetic engineering i.e. the enhancer element is not incorporated into the genome of the plant by an essentially biological means, such as crossing, interbreeding or selective breeding. As a result of such genomic alteration, the engineered plant is different from the related wild-type plant and has a trait that is not naturally found in the wild-type plant. In certain embodiments, the enhancer element(s) is/are stably incorporated into the genome of the plant.

Antioxidants

As the sodium concentration within a cell increases, the sodium ions interfere with metabolic reactions and cause an increase in reactive oxygen species and radicals, such as hydrogen peroxide, OH—and oxygen. These reactive oxygen species and radicals can have various deleterious effects in the cell, such as damaging the DNA. Antioxidants are compounds that inhibit oxidation, which produces reactive oxygen species and radicals. Excess levels of chloride ions can also lead to an increase in reactive oxygen species and radicals. The inventors have advantageously identified that expressing at least one transgene that encodes an antioxidant in a transgenic plant increases salt tolerance of the transgenic plant. The inventors have advantageously identified that operatively linking an enhancer element to a gene of interest that encodes an antioxidant in an engineered plant increases salt tolerance of the engineered plant.

The antioxidant can be a mitochondrial antioxidant or a cytoplasmic antioxidant. In some embodiments, the antioxidant is superoxide dismutase 1 (SODA). In some embodiments, the antioxidant is superoxide dismutase 2 (SOD2). In some embodiments, the antioxidant is cytosolic superoxidase dismutase (SODCC1).

Proteins that can Control the Intracellular Ion Concentration

Proteins that can control the intracellular ion concentration include ion transporters (e.g. a sodium or potassium transporter), hydrogen exporting ATPases, hydrogen exporting pyrophosphatases or protein kinases.

Plants use two strategies to maintain a low cytosolic sodium concentration. Sodium can be excluded from the cytosol to the apoplast or the extracellular space. Alternatively, sodium can be transported to the vacuole where it is stored, which is known as sodium compartmentation. The inventors have identified that exploiting both plasma membrane and tonoplast mechanisms for reducing the cytosolic sodium concentration can advantageously increase the stress tolerance of an engineered plant.

Ion Transporters

Ion transporters can transport ions, such as sodium or potassium across membranes either passively via an ion gradient or actively using energy from various sources—including adenosine triphosphate (ATP), sunlight, and other redox reactions. Ion transporters for use in the invention may be active ion transporters. Active ion transporter are able to remove ions from the cytosol of the plant cell against a concentration gradient.

Plasma membrane-bound sodium/hydrogen antiporters operate to exclude sodium from the cell. SOS1 (Salt Overly Sensitive 1) is a plasma membrane sodium/hydrogen antiporter that extrudes excessive sodium from the cytosol. Studies in several species have now shown that SOS1 is conserved in higher plants including both monocotyledonous and dicotyledonous plants (Liu et al. (2013) Molecular plant, 6, 2, 275-286). SOS1 has also been was previously identified as essential for plant salt tolerance (Shi et al. (2000) PNAS, 97(12): 6896-6901).

Sodium compartmentation can occur in the vacuole or the apoplast. The plant vacuoles play central roles in plant stress responses. The central vacuole, which can occupy more than 80% of the total plant cell volume, is separated from the surrounding cytosol by the tonoplast membrane that controls the passage of inorganic and organic solutes to and from the cytoplasm through a wide range of pumps, carriers, ion channels and receptors (Silva and Geros (2009) Plant Signal Behav (8): 718-726). NHX1 (Na+/H+ antiporter 1) is a tonoplast (vacuolar) sodium/hydrogen ion exchanger.

In some embodiments, the ion transporter is a sodium/hydrogen antiporter. In some embodiments, the ion transporter is a plasma membrane sodium/hydrogen antiporter, such as SOS1. In some embodiments, the ion transporter is a sodium/hydrogen ion exchanger. In some embodiments, the ion transporter is a tonoplast sodium/hydrogen ion exchanger, such as NHX1. The inventors have identified that altering the expression of a tonoplast ion transporter in combination with a plasma membrane ion transporter using enhancer elements can advantageously increase salt tolerance, because sodium can be both excluded and compartmentalized from the cytosol.

Protein Kinases

SOS1 function is regulated by SOS2 (salt overly sensitive 2). SOS2 is a serine/threonine protein kinase. Constitutive expression of SOS2 has been shown to constitutively activate SOS1 allowing for a continued salt transport out of the cell and increase salt tolerance (Huertas et al. (2012) Plant, Cell & Environment, 5, Issue 8/p. 1467-1482). The inventors have identified that altering the expression of a protein kinase using an enhancer element in combination with altering the expression of at least one ion transporter, such as a plasma membrane ion transporter using an enhancer element, can be advantageous, because the protein kinase can activate the ion transporter, which results in improved salt tolerance.

Hydrogen Exporting ATPases and Pyrophosphatases

The inventors have identified that altering the expression of sodium/hydrogen antiporters and hydrogen exporting ATPases and pyrophosphatases in engineered or transgenic plants is advantageous, because it ensures that a pH gradient exists across the plasma membrane and tonoplast. Sodium/hydrogen transporters use the pH gradient generated by hydrogen exporting ATPases and pyrophosphatases to transport sodium out of the cytosol. This pH gradient therefore helps to power sodium transport out of the cytoplasm and thereby improve the salt tolerance of the engineered or transgenic plant. For example, the AHA family are plasma membrane bound hydrogen exporting ATPases (H+-ATPases), which includes AHA1, AHA2 and AHA3. Their primary function is to transport hydrogen ions across the plasma membrane and out of the cell. This generates an electrochemical proton gradient and eventually a proton motive force. This proton motive force acts like a battery—hydrogen ions are forcefully pushed out of the cell, creating the electrochemical gradient that provides the energy to readily exchange hydrogen ions outside of the cell for sodium ions inside the cell.

Hydrogen exporting ATPases and pyrophosphatases are major components of the vacuolar membrane of plant cells (Silva and Geros (2009) Plant Signal Behav (8): 718-726). VHA-A (Vacuolar-type H+-ATPase subunit A1) is a vacuolar ATPase which breaks down ATP to transport hydrogen ions into the vacuole. This creates a strong proton motive force, similar to the SOS1 and AHA3 cycle, across the tonoplast by building up a large amount of hydrogen ions inside the vacuole that can be exchanged for sodium ions. This is believed to advantageously increase the salt tolerance of the plant.

In some embodiments, the protein that can controls the intracellular ion concentration is a hydrogen exporting ATPase or a hydrogen exporting pyrophosphatase. In some embodiments, the protein that can controls the intracellular ion concentration is a hydrogen exporting ATPase. In some embodiments, the hydrogen exporting ATPase is a tonoplast hydrogen exporting ATPase or a plasma membrane hydrogen exporting ATPase.

In certain embodiments, the plasma membrane hydrogen exporting ATPase is AHA3. In certain embodiments, the tonoplast hydrogen exporting ATPase is VHA-A. In other embodiments the tonoplast hydrogen exporting ATPase is VHA-B.

Engineered and Transgenic Plants

In some embodiments the engineered or transgenic plant can be angiosperms. Angiosperms include both monocotyledonous angiosperms and dicotyledonous angiosperms. Monocotyledonous angiosperms include, but are not limited to, cereal crops, such as Zea mays (maize), Oryza sativa (rice), the genus Saccharum (sugar cane), Hordeum vulgare (barley), millet, oat (Avena sativa), Secale cereale (rye), the genus sorghum (sorghum), the genus Triticum (wheat), or any combination thereof. Dicotyledonous angiosperms include vegetable crops, such as Brassica (e.g. Brassica oleracea, kale), Glycine (e.g. Glycine max—soybean), Vigna radiata (mung bean), Chenopodium quinoa (quinoa), Soja, or Solanum (e.g. Solanum tuberosum—tomato).

In certain embodiments, the engineered plant is an engineered rice plant. In certain embodiments, the transgenic plant is a transgenic rice plant. Possible rice varieties that can be used in the invention include: Agostano, Dichroa, Early Sutarsar, Hunan Early Dwarf, Java, Kendzo, Konosu #2, Kurumiwase, Kwanto Wase, Novelli Gigante, Okuro Mochi, Primanychskij, Sensho Tane, Venere Italian Black Rice, Zhe 733, Cho Seun Zo Saeng, Se Zic, Daido, Mamoriaka, Duborskian, Yukihikari, Hayayuki, Truong Giang, MHC-2, TH3-5, Ho Phat 3. In some embodiments, the engineered or transgenic plant is the rice variety Hayayuki, TH 3-5, Truong Giang, MHC2 and Ha Phat 3, Java long, Se Zic, Agostano, Hunan and Dichroa. In certain embodiments, the engineered rice plant is a rice variety Hayayuki engineered plant. In certain embodiments, the transgenic rice plant is a rice variety Hayayuki transgenic plant.

The gene of interest or transgenes described above can originate from any plant species. For example, the gene of interest or transgene may be a rice gene, a maize gene, a sugar cane gene, a barley gene, a millet gene, an oat gene, a rye gene, a sorghum gene, a wheat gene, a kale gene, a soybean gene, a mung bean gene, a quinoa gene, or a tomato gene.

In some embodiments, the transgenic plant comprises a transgene derived from the same species as the transgenic plant. In other embodiments, the transgenic plant comprises a transgene derived from a different species from the transgenic plant, such as an orthologue of a transgene present in the transgenic plant. Where the transgene is derived from the same species as the transgenic plant its insertion as a transgene allows for an additional copy of the gene to be present in the plant and/or the transgene is inserted under the control of a promoter which is different from the endogenous promoter which allows for the expression level or pattern to differ from that of the expression pattern or level of the endogenous gene.

In certain embodiments, the transgenic plant is a transgenic rice plant that comprises transgenes that are rice genes. In some embodiments, the transgenic plant is a transgenic maize plant that comprises transgenes that are maize genes. In some embodiments, the transgenic plant is a transgenic sugar cane plant that comprises transgenes that are sugar cane genes. In some embodiments, the transgenic plant is a transgenic barley plant that comprises transgenes that are barley genes. In some embodiments, the transgenic plant is a transgenic millet plant that comprises transgenes that are millet genes. In some embodiments, the transgenic plant is a transgenic oat plant that comprises transgenes that are oat genes.

In some embodiments, the transgenic plant is a transgenic rye plant that comprises transgenes that are rye genes. In some embodiments, the transgenic plant is a transgenic sorghum plant that comprises transgenes that are sorghum genes. In some embodiments, the transgenic plant is a transgenic wheat plant that comprises transgenes that are wheat genes. In some embodiments, the transgenic plant is a transgenic kale plant that comprises transgenes that are kale genes. In some embodiments, the transgenic plant is a transgenic soybean plant that comprises transgenes that are soybean genes. In some embodiments, the transgenic plant is a transgenic mung bean plant that comprises transgenes that are mung bean genes. In some embodiments, the transgenic plant is a transgenic quinoa plant that comprises transgenes that are quinoa genes. In some embodiments, the transgenic plant is a transgenic tomato plant that comprises transgenes that are tomato genes.

The engineered plants, plant parts and multicellular structures are not produced by a process that involves homologous recombination. The engineered plants, plant parts and multicellular structures and the seeds according to the invention are not produced by an essentially biological process. The transgenic plants, plant parts and multicellular structures are not produced by a process that involves homologous recombination. The transgenic plants, plant parts and multicellular structures and the seeds according to the invention are not produced by an essentially biological process.

In some embodiments, the engineered or transgenic plant is capable of growing in a medium having a salt concentration greater than about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 32 g/L, about 34 g/L, about 36 g/L, about 38 g/L, about 40 g/L, about 42 g/L, about 44 g/L, about 46 g/L, about 48 g/L, about 50 g/L. In certain embodiments, the engineered or transgenic plant is capable of growing in a medium having a salt concentration greater than about 10 g/L, 20 g/L or 35 g/L. In further certain embodiments, the engineered or transgenic plant is capable of growing in a medium having a salt concentration greater than about 35 g/L, because this is greater than ocean salinity.

The engineered and transgenic plants of the invention can have increased salinity tolerance compared to a wild type plant of the same species. In some embodiments, the engineered or transgenic plant has an elevated growth rate in a medium having a particular salt concentration or ECe compared to the growth rate of a wild type plant. The medium that the engineered or transgenic plant can grow in can have an electrical conductivity of a saturated paste extract (ECe) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some embodiments, the medium is liquid (e.g., for hydroponic growth strategies). In some embodiments, the medium is solid (e.g., soil, sand). In some embodiments, the medium is semi-solid.

In some embodiments, the engineered or transgenic plant has a growth rate in a saline medium that is equal to or greater than the growth rate in a non-saline medium. The saline medium can be “slightly saline” (e.g., having an electrical conductivity of the saturated paste extract (ECe) or liquid EC of between 4 and 8 dS/m), “moderately saline” (e.g., having an electrical conductivity of the saturated paste extract (ECe) or liquid EC of between 8 and 16 dS/m), or “severely saline” can be defined as having an electrical conductivity of the saturated paste extract (ECe) or liquid EC of greater than 16 dS/m. The non-saline medium can have an electrical conductivity of the saturated paste extract (ECe) or liquid EC of less than 4 dS/m, less than 3 dS/m, less than 2 dS/m, less than 1.7 dS/m, or less than 1.5 dS/m. In some embodiments, the growth rate in saline medium is at least 5%, 10%, 15%, 20%, 25%, 30%, 35%, 40%, 45%, 50%, 100%, 150%, 200% or more.

In some embodiments, the engineered or transgenic plant has a particular threshold salinity (ECt), or an elevated threshold salinity. In some embodiments, the engineered or transgenic plant has a threshold salinity of at least about 6 dS/m, at least about 6.5 dS/m, at least about 6.7 dS/m, at least about 7 dS/m, at least about 7.5 dS/m, at least about 8 dS/m, at least about 8.5 dS/m, at least about 9 dS/m, at least about 9.5 dS/m, at least about 10 dS/m, at least about 10.5 dS/m, at least about 11 dS/m, at least about 11.5 dS/m, at least about 12 dS/m, at least about 12.5 dS/m, at least about 13 dS/m, at least about 13.5 dS/m, at least about 14 dS/m, at least about 14.5 dS/m, at least about 15 dS/m, at least about 15.5 dS/m; at least about 16 dS/m, at least about 16.5 dS/m, at least about 17 dS/m, at least about 17.5 dS/m; at least about 18 dS/m, at least about 18.5 dS/m; at least about 19 dS/m, at least about 19.5 dS/m, at least about 20 dS/m, at least about 21 dS/m, at least about 22 dS/m, at least about 23 dS/m at least about 24 dS/m, at least about 25 dS/m, at least about 26 dS/m, at least about 27 dS/m, at least about 28 dS/m, at least about 29 dS/m, or at least about 30 dS/m, or more. In some embodiments, the elevated threshold salinity is assessed relative to a same plant species or cultivar without the genome edits. In some embodiments, the threshold salinity is elevated by at least about 1 dS/m, at least about 2 dS/m, at least about 3 dS/m, at least about 4 dS/m, at least about 5 dS/m, at least about 6 dS/m, at least about 7 dS/m, at least about 8 dS/m, at least about 9 dS/m, at least about 10 dS/m, at least about 11 dS/m, at least about 12 dS/m, at least about 13 dS/m, at least about 14 dS/m, or at least about 15 dS/m, or more.

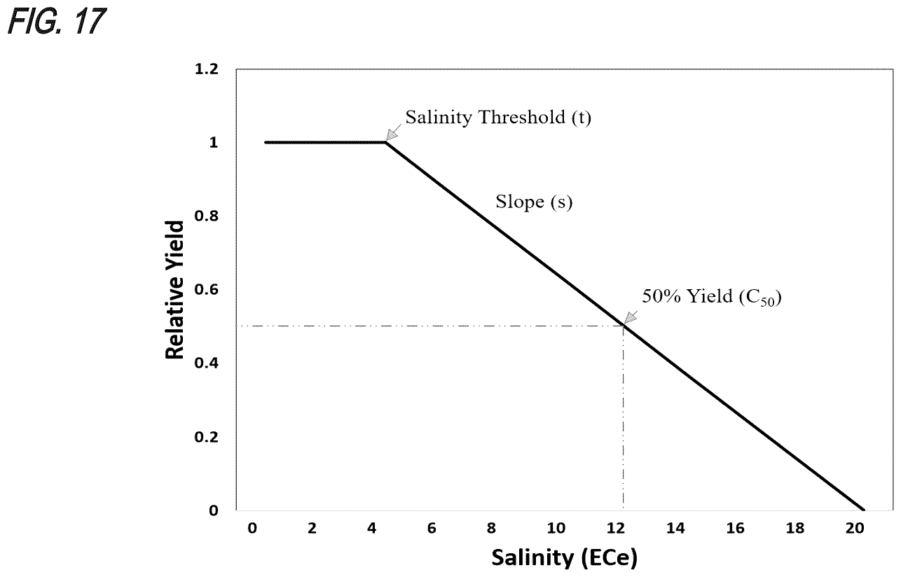

In some embodiments, the engineered or transgenic plant has a particular slope (s) of a yield vs salinity (ECe) plot, or a decreased slope (s) of a yield vs salinity (ECe) plot. In some embodiments, the decreased slope is assessed relative to a same plant species or cultivar without the genome edits. In some embodiments, the slope is decreased by at least about 1% per dS/m, at least about 1.5% per dS/m, at least about 2.0% per dS/m, at least about 2.5% per dS/m, at least about 3.0% per dS/m, at least about 3.5% per dS/m, at least about 4.0% per dS/m, at least about 4.5% per dS/m, at least about 5.0% per dS/m, at least about 5.5% per dS/m, at least about 6.0% per dS/m, at least about 8% per dS/m, or at least about 10% per dS/m, or more. An example of a yield vs salinity (ECe) plot can be seen in FIG. 17.

In some embodiments, the engineered or transgenic plant has a particular starch content as a mature plant, or the seeds or root of the mature plant have can have a particular starch content. In some embodiments, the plant, root, fruit, or seeds of the mature plant can have at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof.

Engineered and Transgenic Plant Parts

The invention also provides part of the engineered or transgenic plants of the invention. For example, the plant part can be a cell, a seed, a leaf, a shoot, a stem or a root. In certain embodiments, the plant part is a seed or a cell. The plant parts such as the seeds, may therefore comprise any of the combinations of genes of interest or transgenes as described above for the engineered or transgenic plants. For example, the seed comprising at least two genes of interest, wherein the first gene encodes a protein that controls the intracellular ion concentration and wherein the second gene encodes an antioxidant, wherein the gene that encodes a protein that controls the intracellular ion concentration is operatively linked to an enhancer element and the gene that encodes an antioxidant is operatively linked to an enhancer element. In other embodiments, the seed comprises at least two genes of interest, wherein the first and the second genes of interest encode proteins that control the intracellular ion concentration, wherein the first genes of interest encodes a plasma membrane protein that controls the intracellular ion concentration and wherein the second genes of interest encodes a tonoplast protein that controls the intracellular ion concentration, wherein the gene that encodes a plasma membrane protein that controls the intracellular ion concentration is operatively linked to an enhancer element and the gene that encodes a tonoplast protein is operatively linked to an enhancer element.

In other embodiments, the seed comprises at least four genes of interest, wherein the seed is a rice seed and wherein the genes of interest encode OsSOS1, OsNHX1, OsHKT1 and OsSODA1 and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element. In other embodiments, the seed comprises at least four genes of interest, wherein the seed is a rice seed and wherein the genes of interest encode OsSOS1, OsNHX1, OsHKT1 and OsAHA3 and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsAHA3 is operatively linked to an enhancer element.

For example, the seed comprising at least two transgenes, wherein the first transgene encodes a protein that controls the intracellular ion concentration and wherein the second transgene encodes an antioxidant.

In other embodiments, the seed comprises at least two transgenes, wherein the first and the second transgenes encode proteins that control the intracellular ion concentration, wherein the first transgene encodes a plasma membrane protein that controls the intracellular ion concentration and wherein the second transgene encodes a tonoplast protein that controls the intracellular ion concentration. In other embodiments, the seed comprises at least four transgenes, wherein the seed is a rice seed and wherein the transgenes encode OsSOS1, OsNHX1, OsHKT1 and OsSODA1.

In some embodiments, the engineered rice seed comprises at least four genes of interest, wherein the genes of interest encode OsSOS1, OsNHX1, OsHKT1 and OsSODA1, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising an amino acid sequence that has at least 95% sequence identity to SEQ ID NO: 92, and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsSODA1 is operatively linked to an enhancer element.

In some embodiments, the engineered rice seed comprises at least four genes of interest, wherein the genes of interest encode OsSOS1, OsNHX1, OsHKT1 and OsAHA3, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsAHA3 gene comprises a polynucleotide sequence that encodes a polypeptide comprising an amino acid sequence that has at least 95% sequence identity to SEQ ID NO: 101, and wherein the gene that encodes OsSOS1 is operatively linked to an enhancer element; wherein the gene that encodes OsNHX1 is operatively linked to an enhancer element; wherein the gene that encodes OsHKT1 is operatively linked to an enhancer element and wherein the gene that encodes OsAHA3 is operatively linked to an enhancer element.

In some embodiments, the engineered rice seed comprises at least four genes of interest, wherein the genes of interest encode OsSOS1, OsNHX1, OsSOS2 and OsVHA-A, wherein the OsSOS1 gene comprises a polynucleotide sequence that encodes a polypeptide comprising the amino acid sequence of SEQ ID NO: 95 and/or 96; wherein the OsNHX1 gene comprises a polynucleotide sequence that encodes a polypeptide comprising the amino acid sequence of SEQ ID NO: 97 and/or 98; wherein the OsSOS2 gene comprises a polynucleotide sequence that encodes a polypeptide comprising the amino acid sequence of SEQ ID NO: 100; wherein the OsVHA-A gene comprises a polynucleotide sequence that encodes a polypeptide comprising the amino acid sequence of SEQ ID NO: 102, wherein the gene that encodes OsSOS1 is operatively linked to an enhancer element; wherein the gene that encodes OsNHX1 is operatively linked to an enhancer element; wherein the gene that encodes OsSOS2 is operatively linked to an enhancer element and wherein the gene that encodes OsVHA-A is operatively linked to an enhancer element.

In some embodiments, the rice seed comprises at least four transgenes, wherein the transgenes encode OsSOS1, OsNHX1, OsHKT1 and OsSODA1, wherein the OsSOS1 transgene comprises a polynucleotide sequence that encodes a polypeptide comprising an amino acid sequence that has at least 95% sequence identity to SEQ ID NO: 95 and/or 96; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92.

In certain embodiments, the engineered rice seed comprises at least eight genes of interest, wherein the at least eight genes of interest encode OsSOS1, OsSOS2, OsAHA3, OsVHA A, OsNHX1, OsHKT1, OsSODA1 and OsSOD2; wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSOD2 gene comprises a polynucleotide sequence that encodes a polypeptide comprising the amino acid sequence of SEQ ID NO: 93; and wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSOD2 is operatively linked to an enhancer element.

In certain embodiments, the rice seed comprises at least eight transgenes, wherein the transgenes encode OsSOS1, OsSOS2, OsAHA3, OsVHA A, OsNHX1, OsHKT1, OsSODA1 and OsSOD2; wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101; wherein the OsVHA A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSOD2 transgene comprises a polynucleotide sequence that encodes a polypeptide comprising an amino acid sequence that has at least 95% sequence identity to SEQ ID NO: 93.

In certain embodiments, the engineered rice seed comprising at least eight genes of interest wherein the at least eight genes of interest encode OsSOS1, OsSOS2, OsAHA3, OsVHA A, OsNHX1, OsHKT1, OsSODA1 and OsSODCC1;
wherein the OsSOS1 gene comprises a polynucleotide sequence that encodes a polypeptide comprising the amino acid sequence of SEQ ID NO: 95 and/or 96; wherein the OsSOS2 gene comprises a polynucleotide sequence that encodes a polypeptide comprising the amino acid sequence of SEQ ID NO: 100; wherein the OsAHA3 gene comprises a polynucleotide sequence that encodes a polypeptide comprising the amino acid sequence of SEQ ID NO: 101; wherein the OsVHA A gene comprises a polynucleotide sequence that encodes a polypeptide comprising the amino acid sequence of SEQ ID NO: 102; wherein the OsNHX1 gene comprises a polynucleotide sequence that encodes a polypeptide comprising the amino acid sequence of SEQ ID NO: 97 and/or 98; wherein the OsHKT1 gene comprises a polynucleotide sequence that encodes a polypeptide comprising the amino acid sequence of SEQ ID NO: 99; wherein the OsSODA1 gene comprises a polynucleotide sequence that encodes a polypeptide comprising the amino acid sequence of SEQ ID NO: 92; wherein the OsSODCC1 gene comprises a polynucleotide sequence that encodes a polypeptide comprising the amino acid sequence of SEQ ID NO: 94; and
wherein the gene that encodes OsSOS1 is operatively linked to an enhancer element; wherein the gene that encodes OsSOS2 is operatively linked to an enhancer element; wherein the gene that encodes OsAHA3 is operatively linked to an enhancer element; wherein the gene that encodes OsVHA A is operatively linked to an enhancer element; wherein the gene that encodes OsNHX1 is operatively linked to an enhancer element; wherein the gene that encodes OsHKT1 is operatively linked to an enhancer element; wherein the gene that encodes OsSODA1 is operatively linked to an enhancer element and wherein the gene that encodes OsSODCC1 is operatively linked to an enhancer element.

In other embodiments, the rice seed comprises at least eight transgenes wherein the transgenes encode OsSOS1, OsSOS2, OsAHA3, OsVHA A, OsNHX1, OsHKT1, OsSODA1 and OsSODCC1; wherein the OsSOS1 transgene comprises a polynucleotide sequence that encodes a polypeptide comprising an amino acid sequence that has at least 95% sequence identity to SEQ ID NO: 95 and/or 96; wherein the OsSOS2 transgene comprises a polynucleotide sequence that encodes a polypeptide comprising an amino acid sequence that has at least 95% sequence identity to SEQ ID NO: 100; wherein the OsAHA3 transgene comprises a polynucleotide sequence that encodes a polypeptide comprising an amino acid sequence that has at least 95% sequence identity to SEQ ID NO: 101;
wherein the OsVHA A transgene comprises a polynucleotide sequence that encodes a polypeptide comprising an amino acid sequence that has at least 95% sequence identity to SEQ ID NO: 102; wherein the OsNHX1 transgene comprises a polynucleotide sequence that encodes a polypeptide comprising an amino acid sequence that has at least 95% sequence identity to SEQ ID NO: 97 and/or 98; wherein the OsHKT1 transgene comprises a polynucleotide sequence that encodes a polypeptide comprising an amino acid sequence that has at least 95% sequence identity to SEQ ID NO: 99; wherein the OsSODA1 transgene comprises a polynucleotide sequence that encodes a polypeptide comprising an amino acid sequence that has at least 95% sequence identity to SEQ ID NO: 92; wherein the OsSODCC1 transgene comprises a polynucleotide sequence that encodes a polypeptide comprising an amino acid sequence that has at least 95% sequence identity to SEQ ID NO: 94.

In some embodiments, the seed is capable of growth in a medium having a salt concentration greater than about 1 gram per liter (g/L), about 2 g/L, about 3 g/L, about 4 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, or about 30 g/L, about 32 g/L, about 34 g/L, about 36 g/L, about 38 g/L, about 40 g/L, about 42 g/L, about 44 g/L, about 46 g/L, about 48 g/L, about 50 g/L. In certain embodiments, the engineered or transgenic plant is capable of growing in a medium having a salt concentration greater than about 10 g/L, 20 g/L or 35 g/L. In further specific embodiments, the engineered or transgenic plant is capable of growing in a medium having a salt concentration greater than about 35 g/L, because this is greater than ocean salinity. The medium can have an electrical conductivity of a saturated paste extract (ECe) or an electrical conductivity (EC) of at least about 1.7 deciSiemens per meter (dS/m), at least about 2 dS/m, at least about 4 dS/m, at least about 6 dS/m, at least about 8 dS/m, at least about 10 dS/m, at least about 12 dS/m, at least about 14 dS/m, at least about 16 dS/m, at least about 18 dS/m, at least about 20 dS/m, at least about 22 dS/m, at least about 24 dS/m, at least about 26 dS/m, at least about 28 dS/m, at least about 30 dS/m, at least about 32 dS/m, at least about 34 dS/m, at least about 36 dS/m, at least about 38 dS/m, at least about 40 dS/m, at least about 42 dS/m, at least about 44 dS/m, at least about 46 dS/m, at least about 48 dS/m, or at least about 50 dS/m. In some embodiments, the medium is liquid (e.g., for hydroponic growth strategies). In some embodiments, the medium is solid (e.g., soil, sand). In some embodiments, the medium is semi-solid. In some embodiments, the seed can have a starch concentration of at least about 20% starch by weight, at least about 25% starch by weight, at least about 30% starch by weight, at least about 35% starch by weight, at least about 40% starch by weight, at least about 45% starch by weight, at least about 50% starch by weight, at least about 56% starch by weight, at least about 60% starch by weight, at least about 65% starch by weight, at least about 70% starch by weight, at least about 75% starch by weight, or at least about 80% starch by weight, or more. The starch can be amylose or a derivative thereof.

Methods of Producing Transgenic Plants

The invention provides methods of making transgenic plants, plant parts or multicellular structures according to the invention, the method comprising the steps of: i) introducing the at least two transgenes as described herein into a cell of a plant, wherein the transgenes stably integrate into the genome of the cell of the plant, and ii) regenerating the cell to form a transgenic plant, a plant part or a multicellular structure from the cell. In some embodiments, the transgenes are introduced into the cell by particle bombardment, Agrobacterium mediated transformation or by protoplast transfection. In certain embodiments, the transgenes are introduced into the cell by particle bombardment.

Programmable nucleases such as Cas endonucleases (e.g., Cas9, Cpf1), transcription activator-like effector nucleases (TALENs), and zinc finger nucleases (ZFNs) allow precise genomic edits to be made. Therefore, in certain embodiments, methods of making transgenic plants, plant parts or multicellular structures according to the invention, comprise the steps of:
(a) inducing callus formation from a seed;
(b) precipitating a polynucleotide sequence, a guide RNA and a nuclease onto a microcarrier; wherein the polynucleotide sequence comprises the at least two transgenes as described herein;
(c) transforming the callus with the microcarriers using particle bombardment to generate a transformed callus wherein the polynucleotide sequence integrates into the genome of the transgenic plant, the plant part or the multicellular structure;
(d) recovering the transformed callus to generate a multicellular structure which comprises the at least two transgenes.

In some embodiments, the multicellular structure is regenerated into a transgenic plant, plant tissue, a plant organ, a plant part, plant reproductive material, or cultured plant tissue comprising one or more plant cells described herein. In certain embodiments the multicellular structure is regenerated into a transgenic plant. In some embodiments, the polynucleotide sequence is stably integrated into the genome of the plant.

In some embodiments, the polynucleotide is RNA, DNA or a plasmid. In certain embodiments, the polynucleotide is DNA. DNA may be used rather than RNA, because it has greater stability both extracellular and within the cytoplasm and nucleus, it is less prone to errors during manufacturing and is less prone to errors during repairing Cas cut sites. DNA may be used rather than a plasmid, because DNA offers stable integration into the genome. DNA can often be integrated with smaller insertions which allows it to remain undetected for many generations and thus become a permanent feature of the new organism and its subsequent generations. When combined with CRISPR-associated (Cas) enzymes, DNA insertions safely integrate an insertion into the host genome with the remarkable absence of any vehicular DNA, plasmid DNA integration or any signatures to indicate a cut has been made in the genome. The safety of using these smaller DNA insertions as opposed to the much larger plasmid structures results precise and accurate insertions producing the exact intended output of the insertion/affected gene compared to some unpredictable consequences of introducing plasmid DNA into a cell.

In some embodiments, the nuclease is a Cas nuclease, Cpf1 nuclease, a TALEN or a zinc finger nucleases. In certain embodiments, the nuclease is Cas9 or Cpf1.

In some embodiments, the transgenic plant, the plant part or the multicellular structure is not produced by a process that involves homologous recombination or are not produced by an essentially biological process.

### EXAMPLES

The following examples are offered for illustrative purposes only, and are not intended to limit the scope of the present invention in any way.

### Example 1—Production of Genetic Inserts for Engineered Rice

Genetic inserts were designed to stably introduce enhancer elements into the genome of rice using Cas9 and Cpf1 nucleases. These enhancer elements were introduced such that they were operatively linked to genes of interest which encode a variety of proteins with different functions including a plasma membrane ion transporter; a plasma membrane hydrogen exporting ATPase, a tonoplast hydrogen exporting ATPase, a tonoplast ion transporter, a potassium transporter, a protein kinase and an antioxidant.

The guide RNAs (gRNAs) used for each of the genes of interest are listed in Table 1. A summary of the enhancer elements and the corresponding genes of interest are listed in Table 2. Cas9 homology arms were added to the 5′ and 3′ end of some of the inserts as summarised in Table 3. The enhancer elements were designed to have two purposes: the first is to increase the expression of the genes of interest in order to provide immediate relief from salt stress. The second is to dilute the gene expression of other stress-induced genes. The stress-induced genes that are being diluted are a collective group that prevent plant growth and prevent the plant metabolism from full functioning when under stress. Without wishing to be bound by any particular theory, by diluting the effective expression of the stress-induced genes this will weaken their initial effect, while the increased expression of the genes of interest into the plants should establish a fair and beneficial salt distribution.

The genes of interest that encode antioxidants were operably linked to the enhancer elements that had the same polynucleotide sequence to ensure that they are expressed in concert. The enhancer elements for the antioxidants were design to ensure that antioxidants were expressed at critical moments in the development of a plant to ‘clean up’ reactive oxygen species, while also providing a heightened baseline level across the lifetime and tissues of the plant. The baseline expression of the antioxidants was achieved by the presence of a TATA box in the enhancer element, which is a constitutive and ubiquitously expressed motif, which encourages plant wide expression. The enhancer element for the antioxidant also includes a gibberellic acid responsive element. Gibberellic acid (GA) promotes expression within the early developmental stages and focuses the expression of the antioxidant genes in the epidermal roots. The presence of an ethylene responsive element in the enhancer element results in the expression of the antioxidant genes through the later developmental stages of the plant, with specific expression during the flowering and fruit development stages. The enhancer element for the antioxidant also contained a DREB2A element, which is a stress-inducible promoter element reactive to ethylene.

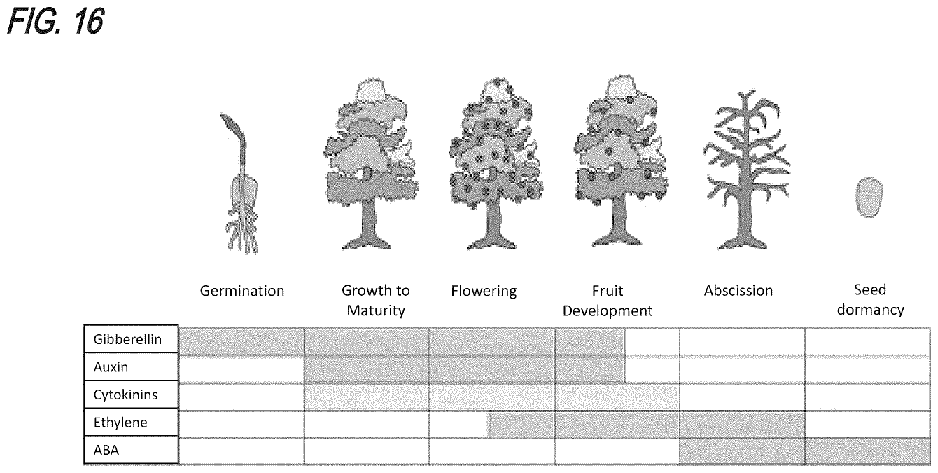

Auxin begins to express after the initial germination and predominantly at the axial locations of both stems and roots (root cap & central root). It continues to express throughout the life cycle of the plant and is supported by ethylene at the flowering and fruit development stage (as seen FIG. 16). Auxin elements were used in the enhancer elements for the SOS1/SOS2/AHA group rather gibberellin, as auxin expresses heavily at the end of roots, especially where roots begin to grow. This can be exploited to create a consistent production of these key proteins that can then implement into the cell membrane of roots as they grow. Over time these roots will slow their growth and the intensity of expression will create a gradient from tip to further back along the root. This allows a gradient to be established that shuttles salt to the tip of the root and propels it out of the root.

TABLE 1
Guide RNAs for each of the genes of interest. The functions of the proteins that these
genes of interest encode and the rice database accession number of the gene are provided.

| Gene | Function | Accession No. | Guide RNA sequence |
|---|---|---|---|
| NHX1 | Tonoplast Na+/H+ | Os0710666900 | TTTACCGGGAATTGTCGAATTAGGC |
| | antiporters | | (SEQ ID NO: 1) |
| VHA-A | Tonoplast H+-ATPase | Os06g0662000 | GTGATCTGACCGCCGG |
| | | | (SEQ ID NO: 2) |
| SOS1 | Na+/H+ antiporter | Os12g0641100 | TTTGCCTTGGATTTTCCACTTGCA |
| | | | (SEQ ID NO: 3) |
| SOS2 | Serine/threonine protein | Os06g0606000 | GAGTTGGATTCGTGGCCGCG CGG |
| | kinase | | (SEQ ID NO: 4) |
| AHA3 | Plasma membrane H+- | Os12g0638700 | TGGTGGGAGATCCATCTGCGAGG |
| | ATPase | | (SEQ ID NO: 5) |
| HKT1 | High-affinity K+ | Os06g0701700 | TTTCTCATACTCGTTGGCTCGTTGC |
| | transporter | | (SEQ ID NO: 6) |
| SODA1 | mitochondrial | Os05t0323900 | CCCATCCTCCAGTGCTACGG (SEQ ID |
| | mangansese superoxide | | NO: 7) |
| | dismutase | | |
| SODCC1 | cytoplasmic and ECM | Os03g0351500 | TTTAGGACCTCTAGAGGCTACTTCTG |
| | CuZn superoxide | | CC (SEQ ID NO: 8) |
| | dismutase | | |
| SOD2 | cytoplasmic CuZn | Os03g0219200 | GCGAAGCGGCAGAGAATGGCAGGGA |
| | superoxide dismutase | | AA (SEQ ID NO: 9) |

TABLE 2
The sequences for the enhancer elements introduced to the 5′ end of
the genes of interest

| Gene | Promoter elements | Enhancer element sequence |
|---|---|---|
| NHX1 | DREB.GA.TAF.TATATA | GCCGACCCTTTGGCCACGTGGCAATATATA |
| | | (SEQ ID NO: 10) |
| VHA-A | TATA/TAF-1 + ETH | GCCGACGCCACGTGGCAAGCCGCCTATATA |
| | | (SEQ ID NO: 11) |
| SOS1 | AUX.ETH.E2F.DREB.E2F | TGTCTCGCCGCCGCGGGAAA |
| | | (SEQ ID NO: 12) |
| SOS2 | AUX/ETH + E2F-1 (*2) + | TGTCTCGCCGCCGCGGGAAAAGCCGACGCGGGA |
| | TATA/DREB?? | AAA (SEQ ID NO: 13) |
| AHA3 | AUX/ETH + E2F-1 (*2) + | TGTCTCGCCGCCGCGGGAAAAGCCGACGCGGGA |
| | TATA/DREB?? | AAA (SEQ ID NO: 14) |
| HKT1 | DREB2Ax2 + E2F-1 | GCTCAAGCCGACGCGGGAAAGGCCGACGCCGCC |
| | | GCCGAC (SEQ ID NO: 15) |
| SODA1 | GA + TATA + DREB2A + ETH | CCTTTGTATATAGCCGACGCCGACGCCGCC |
| | | (SEQ ID NO: 16) |
| SODCC1 | GA + TATA + DREB2A + ETH | CCTTTGTATATAGCCGACGCCGAC |
| | | (SEQ ID NO: 17) |
| SOD2 | GA + TATA + DREB2A + ETH | CCTTTGTATATAGCCGACGCCGACGCCGCC |
| | | (SEQ ID NO: 18) |

TABLE 3
The 5′ and 3′ sequences of the DNA inserts. The residues that are in lower
case were included in the design to ensure that the gene was in frame.
The / denotes residues that form the 4-5 base pair overhang for the Cpf1
nucleases

| Gene | Enzyme | Location | DNA Inserts COMPLETE w/homology arms |
|---|---|---|---|
| NHX1 | Cpf1 | 5′ | GCAT/GCCGACCCTTTGGCCACGTGGCAATATATAtt |
| | | | (SEQ ID NO: 19) |
| | | 3′ | ATCGaaTATATATTGCCACGTGGCCAAAGGGTCGGC |
| | | | (SEQ ID NO: 20) |
| VHA-A | Cas9 | 5′ | CGAGAGAGCCGTCTCCCTCTTCGCTTCTCCTCTCCCCCCCG |
| | | | TGATCTGACGCCGACGCCACGTGGCAAGCCGCCTATATAC |
| | | | GCCGGCGACGACCCCCTCCCACCACCCGCCGCCGCCGCC |
| | | | GCGCCCCCGC (SEQ ID NO: 21) |
| SOS1 | Cpf1 | 5′ | ACTTaa |
| | | | /TGTCTCGCCGCCGCGGGAAAAGCCGACGCGGGAAAA |
| | | | (SEQ ID NO: 22) |
| | | 3′ | AAGT..TTTTCCCGCGTCGGCTTTTCCCGCGGCGGCGAGACA |
| | | | tt (SEQ ID NO: 23) |
| SOS2 | Cas9 | 5′ | CGGCGGCGTCGTCGTCTTCCTCCTCTTCCCCCCGAGTTGG |
| | | | ATTCGTGGCCTGTCTCGCCGCCGCGGGAAAAGCCGACGCG |
| | | | GGAAAACGGCGGATGGGAGGGGAGGAGGGAATGGCGGCG |
| | | | GGGAGGAAGAAGCGGGT (SEQ ID NO: 24) |
| AHA3 | Cas9 | 5′ | GATACAGGTAGAGAGAGGTGAGAAGGCAGTGGTGGGAGAT |
| | | | CCATCTTGTCTCGCCGCCGCGGGAAAAGCCGACGCGGGAA |
| | | | AAGCGAGGTGCCTCCATGGCTGAGAAGGAGGGCAACCTCG |
| | | | ACGCCGTCCTCAAGGA (SEQ ID NO: 25) |
| HKT1 | Cpf1 | 5′ | GCTCAAGCCGACGCGGGAAAGGCCGACgccgccGCCGAC |
| | | | (SEQ ID NO: 26) |
| | | 3′ | GAGC..GTCGGCGGCGGCGTCGGCCTTTCCCGCGTCGGCtt |
| | | | (SEQ ID NO: 27) |
| SODA1 | Cas9 | 5′ | ACCTGCCACTCGCCCACTCCTCCTCCTCCCCCATCCTCCAG |
| | | | TGCCTTTGTATATAGCCGACGCCGACGCCGCCCTACGGTG |
| | | | TCACGCAGCCATGGCGCTCCGCACGCTGGCCTCGAGGAAA |
| | | | ACC (SEQ ID NO: 28) |
| SODCC1 | Cpf1 | 5′ | CTGCaaCCTTTGTATATAGCCGACGCCGACGCCGCC |
| | | | (SEQ ID NO: 29) |
| | | 3′ | gcagGGCGGCGTCGGCGTCGGCTATATACAAAGGtt |
| | | | (SEQ ID NO: 30) |
| OsSOD2 | Cpf1 | 5′ | CCTTTGTATATAGCCGACGCCGACGCCGCCggGCGA |
| | | | (SEQ ID NO: 31) |
| | | 3 | CCGGCGGCGTCGGCGTCGGCTATATACAAAGGtcgc |
| | | | (SEQ ID NO: 32) |

### Example 2—Production of Engineered Plants

Step 1—Callus Induction

Seeds were de-hulled by hand or using tweezers. The surface of the seeds was then sterilized with 75% ethanol for 1 min, followed by 2.5% sodium hypochlorite for 15 mins and then washed with distilled water 8 times. The seeds husks were then autoclaved. The seeds were then placed on rice Callus Induction Media. The components of this media are listed in Table 4 below. The seeds were incubated in the dark for 14 hrs at 25° C. This produced embryogenic calli, which looked white or yellowish and had a compact & nodular structure.

TABLE 4
The components of Rice Callus Induction Media
Rice Callus Induction Media

| 4.3 | g L−1 | Murashige & Skoog Basal Salts and Vitamins |
|---|---|---|
| 30 | g L−1 | Maltose |
| 0.3 | g L−1 | Casein Hydrolysate |
| 0.6 | g L−1 | L-Proline |
| 3 | mg L−1 | 2,4-Dichlorophenoxyacetic acid (2,4-D) |
| 0.25 | mg L−1 | 6-Benzylaminopurine (BAP) |
| 3 | g L−1 | Phytagel |
| 50 g L−1/90 mg L−1 | | Kanamycin/Cefotaxime (either antibiotic) |
| 20 | mg L−1 | Carbendazim (antifungal) |

After 14 days the embryogenic calli were cut from the seed and further split into equal segments no larger than 5 mm in diameter. These calli were transferred onto fresh on Rice Callus Induction Media and incubated in the same conditions (24 h dark @ 25° C.) for 4 days.

Step 2—Particle Bombardment

The DNA inserts designed in Example 1 were transformed into rice using partial bombardment. Each particle bombardment consisted of a maximum number of 4 DNA inserts per bombardment. Therefore, to bombard the embryonic calli with eight DNA inserts multiple sets of bombardments were required. The embryogenic calli were bombarded with the following sets of DNA inserts, which comprise the sequences as described in Table 3 to produce engineered plants as summarized in the table below. The variety of rice used to create the embryogenic calli is also indicated in the table.

### Experiment 1

TABLE 5
The combinations of genes of interest targeted in each rice plant.

| Plant | Genes of interest |
|---|---|
| Genotype 1 | OsNHX1, OsSOS1, OsHKT1, OsSODA1 |
| Genotype 2 | OsNHX1, OsSOS1, OsHKT1, OsSODA1 |
| Genotype 9 | OsNHX1, OsSOS1, OsHKT1, OsSODA1 |
| Genotype 10 | OsNHX1, OsSOS1, OsHKT1, OsSODA1 |

### Experiment 2

SET ONE: NHX1, SOS1, AHA3 and HKT1
SET TWO: VHA-A, SOS2, SODA1 and SOD2

TABLE 6
The combinations of genes of interest targeted in each
rice plant. The rice variety used is also indicated.

| Plant | | |
|---|---|---|
| Sample | Rice variety | Genes of interest |
| A | Hayayuki | OsNHX1, OsSOS1, OsAHA3, OsHKT1 |
| B | Hayayuki | OsNHX1, OsSOS1, OsAHA3, OsHKT1 |
| C | Hayayuki | OsNHX1, OsVHA-A, OsSOS1, OsSOS2, |
| | | OsAHA3, OsHKT1, OsSODA1, OsSOD2 |
| D | Truong Giang | OsNHX1, OsSOS1, OsAHA3, OsHKT1 |
| E1 | Hayayuki | OsNHX1, OsVHA-A, OsSOS1, OsSOS2, |
| | | OsAHA3, OsHKT1, OsSODA1, OsSOD2 |
| F | Hayayuki | OsNHX1, OsVHA-A, OsSOS1, OsSOS2, |
| | | OsAHA3, OsHKT1, OsSODA1, OsSOD2 |
| I | Hayayuki | OsNHX1, OsSOS1, OsAHA3, OsHKT1 |
| G | Hayayuki | OsNHX1, OsVHA-A, OsSOS1, OsSOS2, |
| | | OsAHA3, OsHKT1, OsSODA1, OsSOD2 |
| 1 | Hayayuki | OsNHX1, OsVHA-A, OsSOS1, OsSOS2, |
| | | OsAHA3, OsHKT1, OsSODA1, OsSOD2 |
| 9 | Hayayuki | OsNHX1, OsVHA-A, OsSOS1, OsSOS2, |
| | | OsAHA3, OsHKT1, OsSODA1, OsSOD2 |

In order for the DNA inserts to stably integrate into the genome they need to be bombarded in combination with a programmable nuclease (Cas9 or Cpf1) and at least one RNA guide as shown in Table 1. Cas9 requires two RNA guides, crRNA and tracrRNA to stably insert the DNA insert that comprises the enhancer element into the plant genome, whereas Cpf1 requires only a single guide RNA. The ratio of nuclease enzyme to RNA guide to genetic DNA insert was 1:1:2 per gene. The DNA/enzyme mixture was created as outlined below.

| | Per bombardment | |
|---|---|---|
| Enzyme (Cpf1 or Cas9) | 0.5 | μl (5 ug) |
| RNA Guide 1 | 0.5 | μl |
| RNA Guide 2 | 0.5 | μl |
| RNA Guide 3 | 0.5 | μl |
| RNA Guide 4 | 0.5 | μl |

Incubated 15 mins at room temperature

| | | |
|---|---|---|
| DNA Insert 1 | 1 | μl |
| DNA Insert 2 | 1 | μl |
| DNA Insert 3 | 1 | μl |
| DNA Insert 4 | 1 | μl |

| | |
|---|---|
| Buffer | up to 20 ul |

The DNA/enzyme mixture was precipitated onto gold particles by mixing the gold particles with the DNA/enzyme mixture, spermidine, and calcium chloride. The resulting mixture was incubated on ice for 10 minutes. The DNA coated particles were centrifuged, the supernatant removed and replaced with 75% ethanol, and the gold particles were re-suspended.

The embryogenic calli were placed on a petri dish with rice osmotic medium for 4 hours before bombardment took place. The components of the rice osmotic medium are summarised in Table 7 below. Alternatively, the calli were transferred to Whatman™ filter paper and desiccated for 24 hr in the dark at 25° C. The calli were then transferred to a gene gun system, where under a vacuum pressure of at least −5 Hg, 10 μl of prepared DNA-coated gold particles are fired at least 1100 psi.

After bombardment the calli were placed on rice Osmotic Medium at 26° C. in the dark for 16 hr-20 hr. The calli were then transferred to rice Callus Induction Media and incubated in the dark for 7-14 days at 25° C.

TABLE 7
The components Rice osmotic medium and Rice callus induction media

| Rice Osmotic Media (ROM) | | | Rice Callus Induction Media (RCIM) | | |
|---|---|---|---|---|---|
| 4.3 | g L−1 | Murashige & Skoog Basal | 4.3 | g L−1 | Murashige & Skoog Basal Salts |
| | | Salts and Vitamins | | | and Vitamins |
| 30 | g L−1 | Maltose | 30 | g L−1 | Maltose |
| 90 | g L−1 | Mannitol | 0.3 | g L−1 | Casein Hydrolysate |
| 0.3 | g L−1 | Casein Hydrolysate | 0.6 | g L−1 | L-Proline |
| 0.6 | g L−1 | L-Proline | 3 | mg L−1 | 2,4-Dichlorophenoxyacetic acid |
| | | | | | (2,4-D) |
| 3 | mg L−1 | 2,4- Dichlorophenoxyacetic | 0.25 | mg L−1 | 6-Benzylaminopurine (BAP) |
| | | acid (2,4-D) | | | |
| 0.25 | mg L−1 | 6-Benzylaminopurine (BAP) | 50 g L−1/90 mg L−1 | | Kanamycin/Cefotaxime (either |
| | | | | | antibiotic) |
| 3 | g L−1 | Phytagel | 20 | mg L−1 | Carbendazim (antifungal) |
| 50 mg L−1/90 mg L−1 | | Kanamycin/Cefotaxime | | | |
| | | (either antibiotic) | | | |
| 20 | mg L−1 | Carbendazim (antifungal) | | | |

Step 3—Regeneration of Embryogenic Calli

The calli were then transferred to ice Regeneration Media I (RRM I) and incubated under light conditions (16 h light:8 h dark at 25° C.) for one to three weeks. After around 7 days green spots started to appear on some calli, which developed into shoot and root growth to form a platelet. After 3 weeks, or before the plantlets got too big for transfer, the platelets and remaining calli were transferred onto Rice Regeneration Media II (as described in Table 8 below) and incubated under light conditions (16 h light:8 h dark at 25° C.).

TABLE 8
The components of Rice Regeneration Media I and II)

| Rice Regeneration Media I (RRM I) | | | Rice Regeneration Media II (RRM II) | | |
|---|---|---|---|---|---|
| 4.3 | g L−1 | Murashige & Skoog Basal Salts | 4.3 | g L−1 | Murashige & Skoog Basal Salts |
| 30 | g L−1 | Maltose | 30 | g L−1 | Maltose |
| 2 | g L−1 | Casein Hydrolysate | 100 | mg L−1 | Myo-inositol |
| 30 | g L−1 | Sorbitol | 60 | mg L−1 | Calcium Silicate |
| 0.5 | mg L−1 | Naphthaleneacetic acid (NAA) | 3 | g L−1 | Gelzan |
| 1.5 | mg L−1 | Kinetin | 50 | mg L−1 | Cefotaxime or Vancomycin |
| 0.5 | mg L−1 | BAP | 20 | mg L−1 | Carbendazim (antifungal) |
| 0.5 | mg L−1 | TDZ | | | |
| 4 | g L−1 | Phytagel | | | |
| 50 | mg L−1 | Cefotaxime or Vancomycin | | | |
| 20 | mg L−1 | Carbendazim (antifungal) | | | |

In order to generate more root tissue growth the platelets were transferred Rice Root Proliferation Media (RRPM) (described in Table 9) and incubated under light conditions (16 h light:8 h dark at 25° C.) until the root mass was sufficient to ensure healthy plant growth.

TABLE 9
The components of Rice Root Proliferation Media
Rice Root Proliferation Media (RRPM)

| | | |
|---|---|---|
| 2.15 | g L−1 | Murashige & Skoog Basal Salts and Vitamins |
| 20 | g L−1 | Maltose Note don't add sugar for growth in hydroponics or with bacteria |
| 60 | mg L−1 | Calcium Silicate |
| 2 | g L−1 | Gelzan Note - Gelzan & Phytogel are interchangeable |
| 50 | mg L−1 | Vancomycin/Cefotaxime (either antibiotic) dissolved in distilled water |
| 20 | mg L−1 | Carbendazim (antifungal) dissolved in methanol/ethanol |

### Example 3—Optimisation of Callus Induction Rates

The callus induction rates for different rice varieties was tested using the methods described in Example 2. The effect of different concentrations of the hormones 2,4-dichlorophenoxyacetic acid (2,4-D) and 6-benzylaminopurine (BAP) on callus induction rates was investigated.

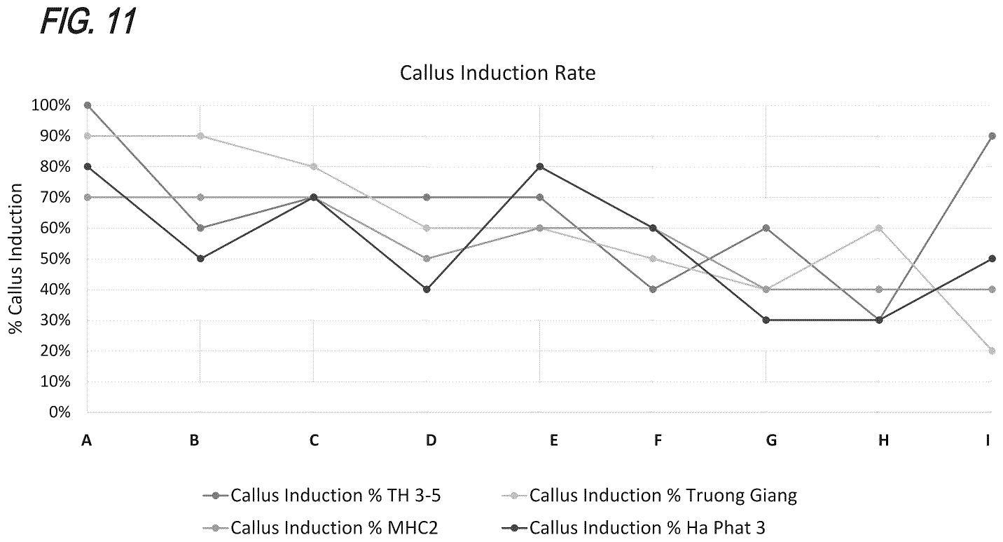

Four different rice varieties were tested: TH 3-5, Truong Giang, MHC2 and Ha Phat 3. The calli were induced as described in step 1 of Example 2. The concentrations of 2,4-D and BAP tested in the rice callus induction media are summarised in Table 10 below. As shown in FIG. 11, calli were successfully produced from all four rice varieties. Media comprising 2 mg/L of 2,4-D and 0.1 mg/I of BAP produced the highest induction rates.

TABLE 10
The concentrations of 2,4-D and BAP tested
in the Rice callus induction media

| | | 2,4 D mg/L | | |
|---|---|---|---|---|
| Media Chart | | 2 | 2.5 | 3 |
| BAP mg/L | 0.1 | A | B | C |
| | 0.25 | D | E | F |
| | 0.5 | G | H | I |

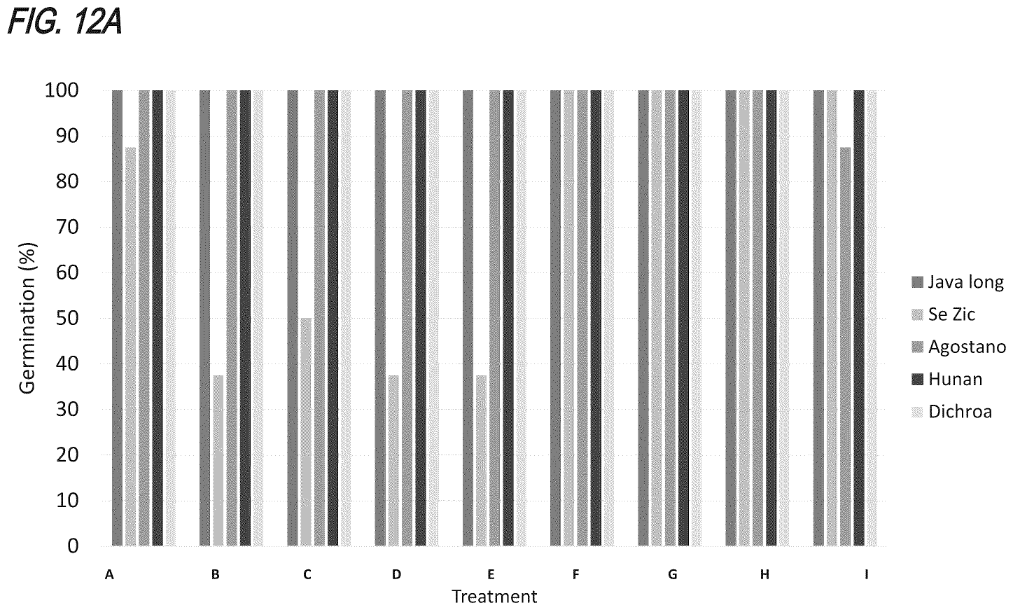

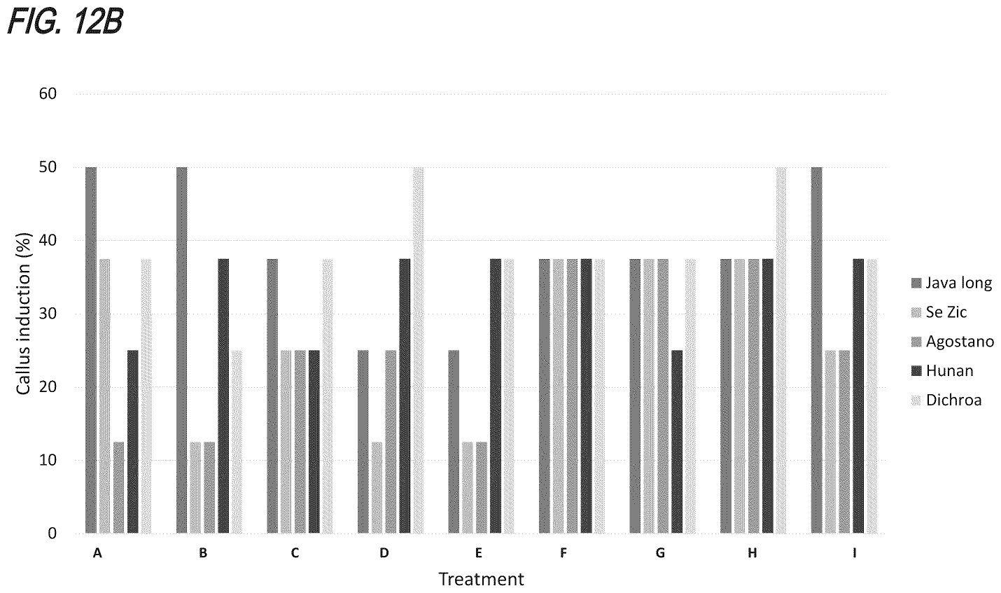

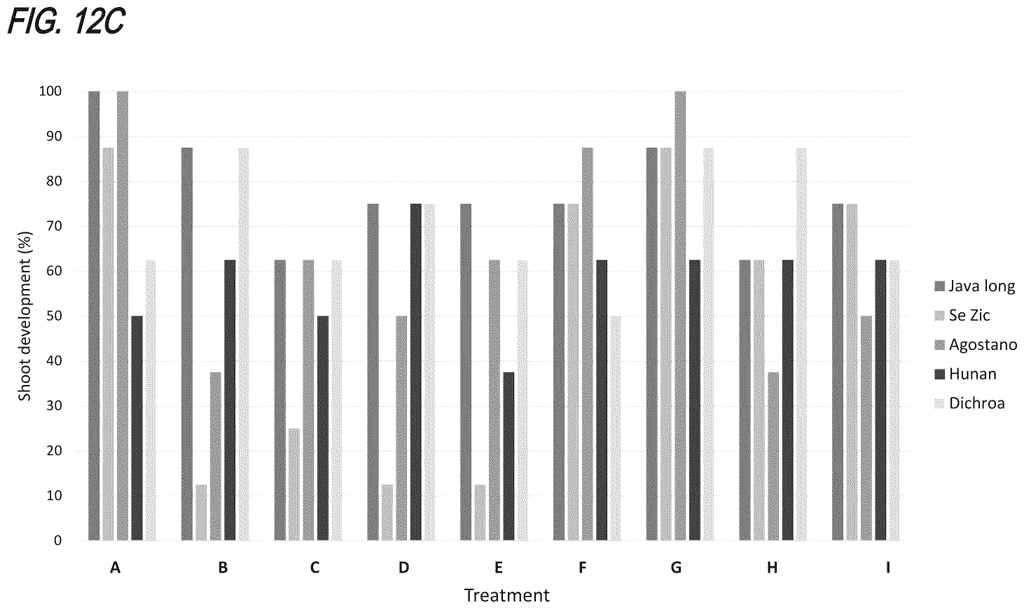

The callus induction rate in five further rice varieties were tested: Java long, Se Zic, Agostano, Hunan and Dichroa. FIG. 12A demonstrates the percentage germination of each of the rice varieties and FIG. 12B demonstrates the percentage of callus induction. These data demonstrate that calli could be produced from all of the varieties tested. FIG. 12C demonstrates that shoot development from these induced calli can also occur all of the varieties tested. These data demonstrate the utility of the callus induction methods for different plant varieties.

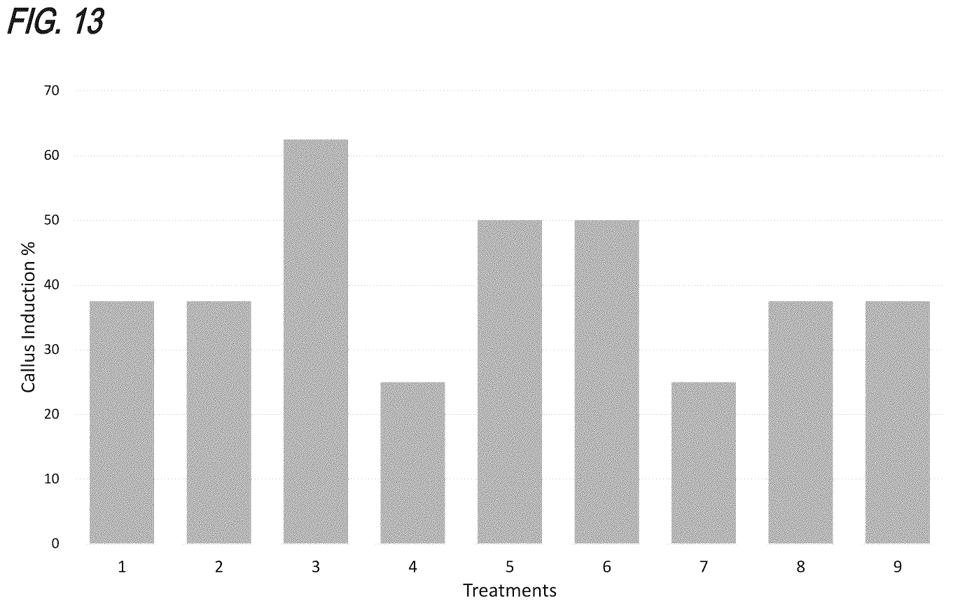

The rate of callus induction was also determined for the rice variety Hayayuki. Calli were induced as described in step 1 of Example 2. The concentrations of 2,4-D and BAP tested in the Rice callus induction media are summarised in Table 11 below. FIG. 13 demonstrates that all of the treatments tested resulted in calli induction of Hayayuki. The combination of 3 mg/L of 2,4-D and 0.1 mg/I of BAP produced the highest induction rates.

TABLE 11
The concentrations of 2,4-D and BAP
tested in the Rice callus induction media

| | | | | | | | | | |
|---|---|---|---|---|---|---|---|---|---|
| BAP | 0.10 | 0.10 | 0.10 | 0.25 | 0.25 | 0.25 | 0.50 | 0.50 | 0.50 |
| (mg/L) | | | | | | | | | |
| 2,4-D | 2.00 | 2.50 | 3.00 | 2.00 | 2.50 | 3.00 | 2.00 | 2.50 | 3.00 |
| (mg/L) | | | | | | | | | |
| Treatment | 1 | 2 | 3 | 4 | 5 | 6 | 7 | 8 | 9 |

### Example 4—Analysis of Regenerated Engineered Plants

The engineered plants produced in Example 2 were analysed using qRT-PCT to determine the expression levels of certain genes compared to wild type plants.

RNA Extraction

Root (5-10 mg) and shoot (˜15 mg) tissue were collected from the regenerated plants from experiment 1 in Example 2 and flash frozen in liquid nitrogen and stored at −80° C. until required. In addition, samples were collected which contained tissue from the whole regenerated engineered plant from experiment 2 in Example 2. These samples predominantly contained leaf tissue.

These samples were subsequently ground in a mortar and pestle with liquid nitrogen. Total RNA was extracted using a GeneJet Plant RNA Purification Kit or Qiagen Plant RNEasy kit, following the manufacturer's protocol. The concentration of the RNA was analysed using a NanoDrop OneC microvolume UV-Vis Spectrophotometer. All the extracted RNAs with a 260/230 ratio of above 2.0 (2.01-2.14) were used for downstream processes. The DNA was removed from the sample by treating with RQ1 RNase-Free DNase at 37° C. for 30 minutes. The DNase was then inactivated by incubating the mixtures at 65° C. for 10 minutes.

ciRT-PCR Analysis

qRT-PCR was used to analyze the gene expression of various salt tolerance genes within the engineered plant. Two sets of probe/dye sets were tested. Both sets included a 5th primer/probe of the housekeeping gene Actin to act as a control.
SET ONE: NHX1, SOS1, AHA3 and HKT1
SET TWO: VHA-A, SOS2, SODA1 and SOD2/SODCC1

The probes attached to each gene were: FAM: NHX1 and VHA-A; SUN: SOS1 and SOS2; ROX: AHA3 and SODA1; and Cy5′: HKT1, SODCC1 and SOD2. A mixture of 12.5 μl iTaq Reaction Mix, 0.5 μl iScript Reverse, 5×1 μl each of a forward and reverse primer for each genes of interest (provided in table X), 5× 0.8 μl Fluorogenic Probe and 1 μl of RNA was placed in a PCT machine with the following cycle: 1 cycle 50° C. for 30 mins, 1 cycle 94° C. for 2 mins, 40 cycles 94° C. for 15 secs, 59° C. for 15 secs, 68° C. for 15 secs, 1 cycle 68° C. for 5 mins. The primers used for each genes of interest were:

NHX1
(SEQ ID NO: 50)
TGCAATTGGAGCCATCTTTTCTGCG
VHA-A
(SEQ ID NO: 51)
AATGCCTGCGGATAGTGGTTACCC
SOS1
(SEQ ID NO: 52)
TGTTACATTCCCTCAGGTGCTTCGTG
SOS2
(SEQ ID NO: 53)
TGTCACCAGCAACCTTTCGAACATCA
AHA3
(SEQ ID NO: 54)
TGGCAATTGGAAAAGAAACAGGGCG
HKT1
(SEQ ID NO: 55)
TGGGAATGTAGGGCTATCCACTGGT
SODA1
(SEQ ID NO: 56)
CCAAAATCCTCATCAATGGCCCAGC
SODCC1
(SEQ ID NO: 57)
ACTGGGCCACACTACAATCCTGC
SOD2
(SEQ ID NO: 58)
ACTACAACTCACAGGATGCAGCAGC

The fluorescence from probes was analyzed using Bio-Rad CFX Maestro software. The relative transcript abundances of genes in regenerated independent-event Rice plants were analyzed compared to the Cq values in wild type plants. All reactions were performed with two technical replicates per sample.

Transcript levels were first analysed relative to Actin and/or 25S ribosomal RNA (25S rRNA) housekeeping genes, with the design based on Rice genome sequence to get the ΔCt values. The relative fold-change gene expressions in regenerated independent-event Rice plants were then analyzed relative to wild type by calculating the ΔΔCt values.

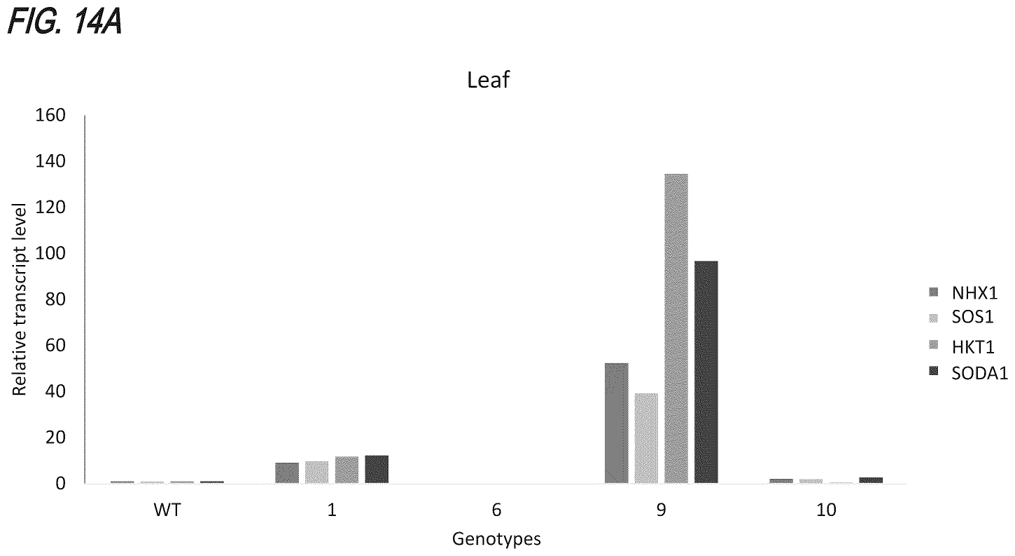

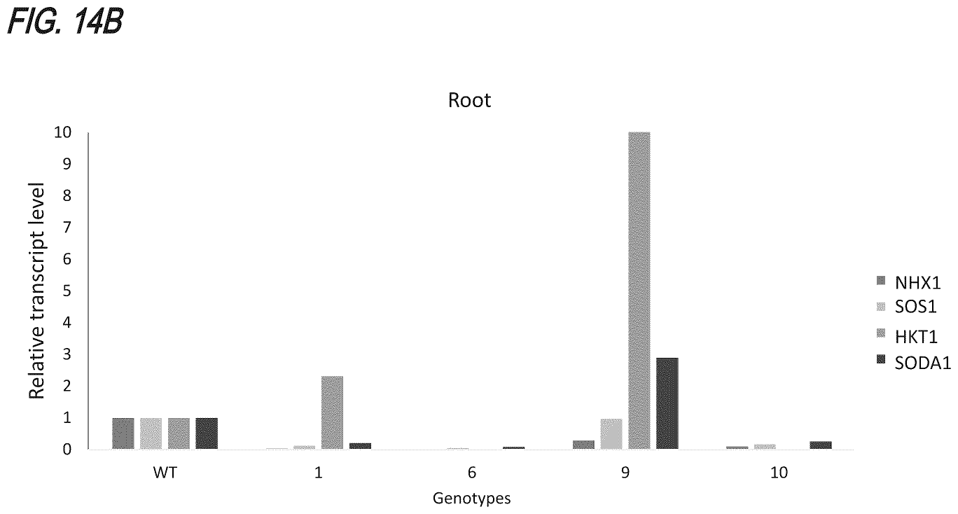

FIG. 14A and FIG. 14B demonstrate that the RNA expression levels of NHX1, SOS1, HKT1 and SODA1 in either the shoot or the root of the engineered rice were significantly higher than compared to the wild type rice. These data demonstrate that the RNA expression levels of NHX1, SOS1, HKT1 and SODA1 were increased compared to a wild type plant. The expression level of the genes of interest varied between the root and the shoot. For example, NHX1 had a higher level of expression in the leaf compared to the root.

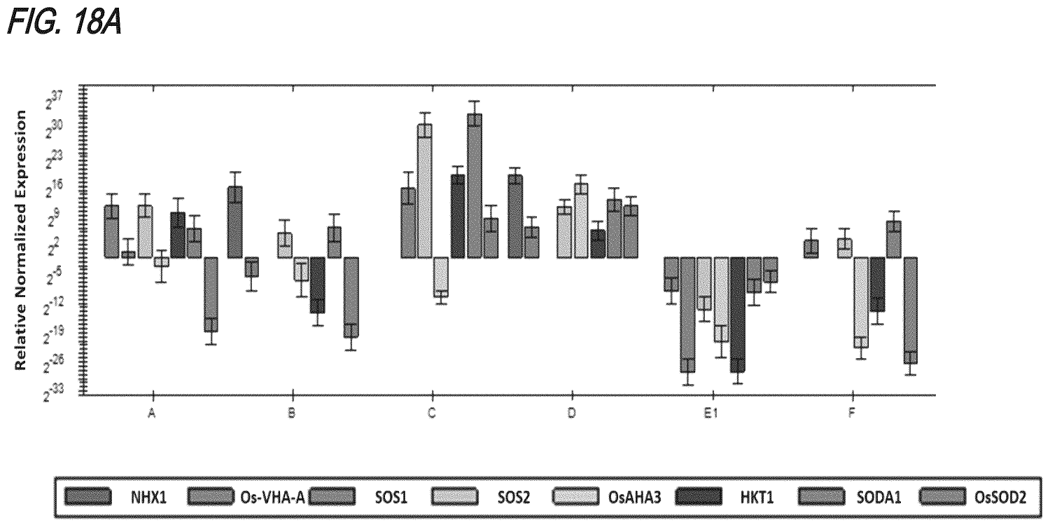

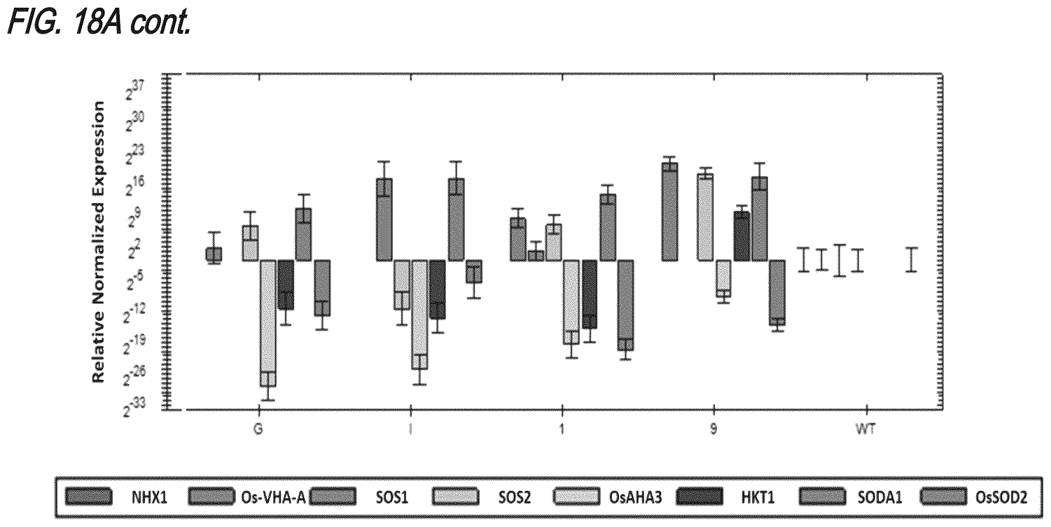

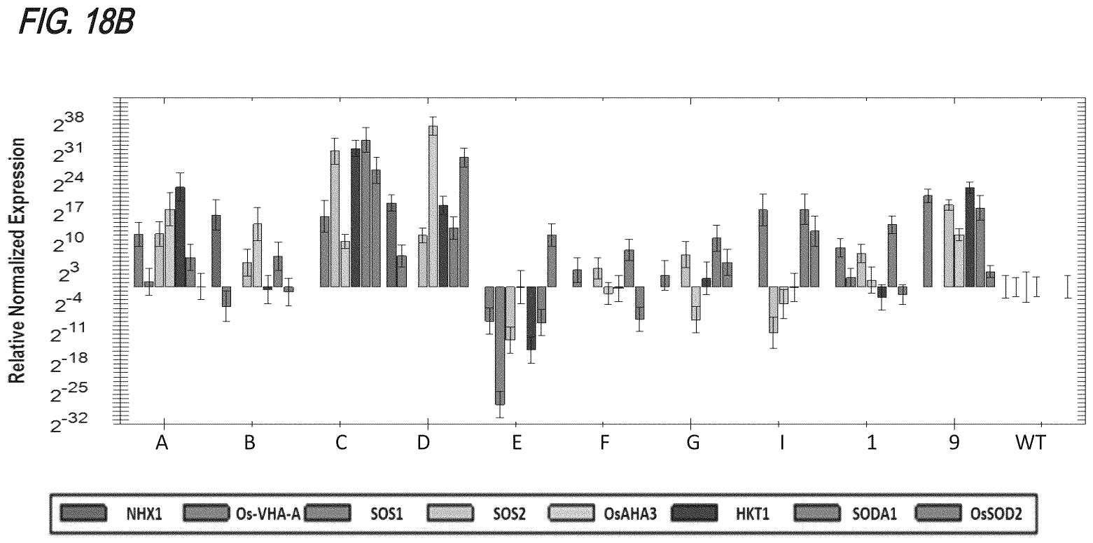

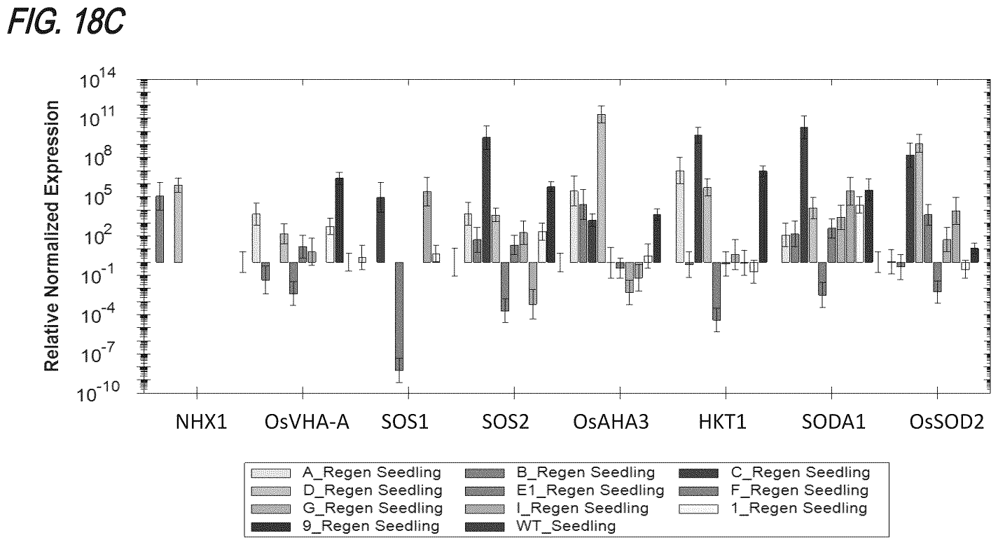

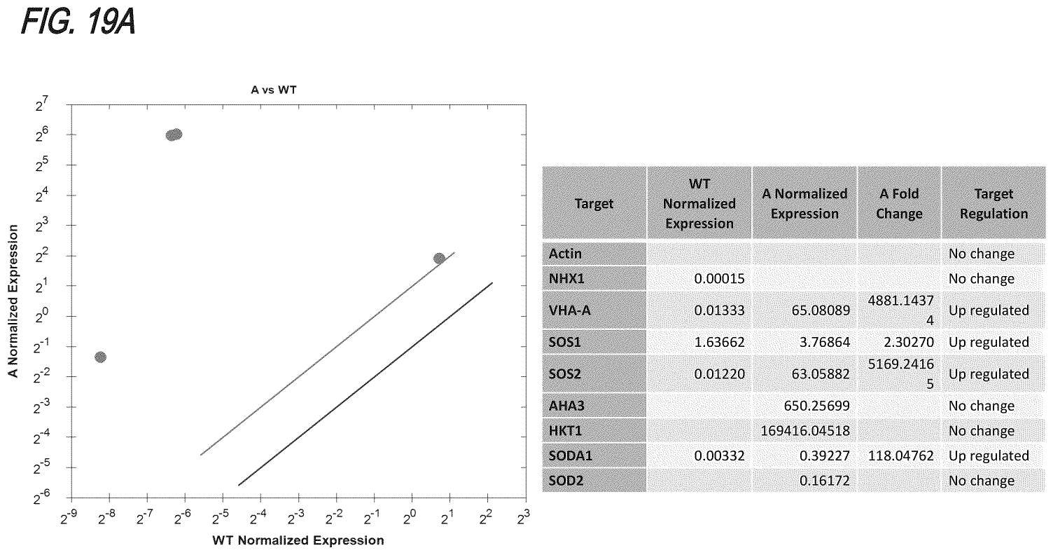

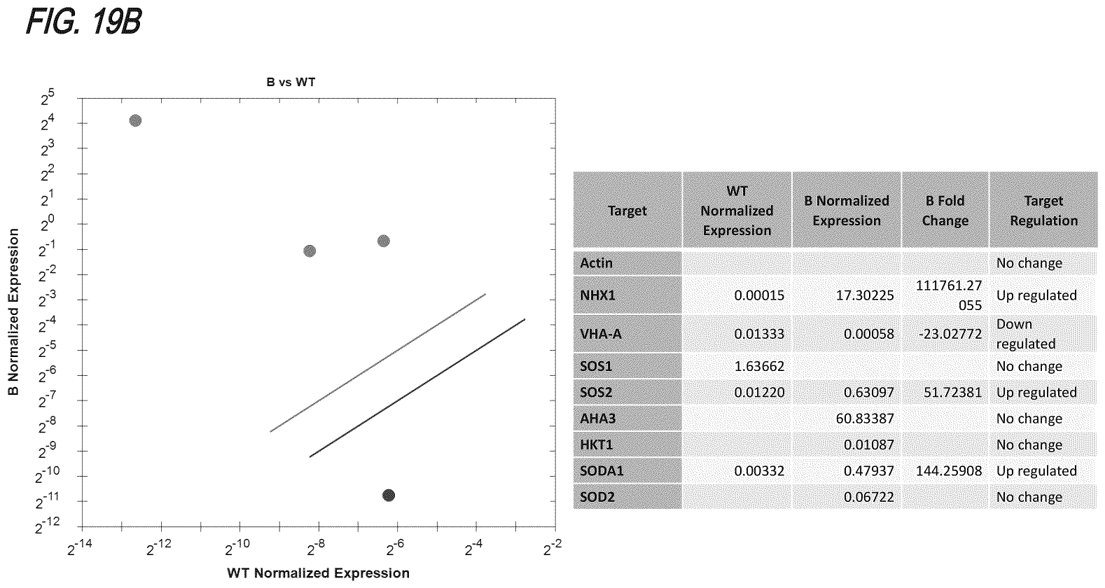

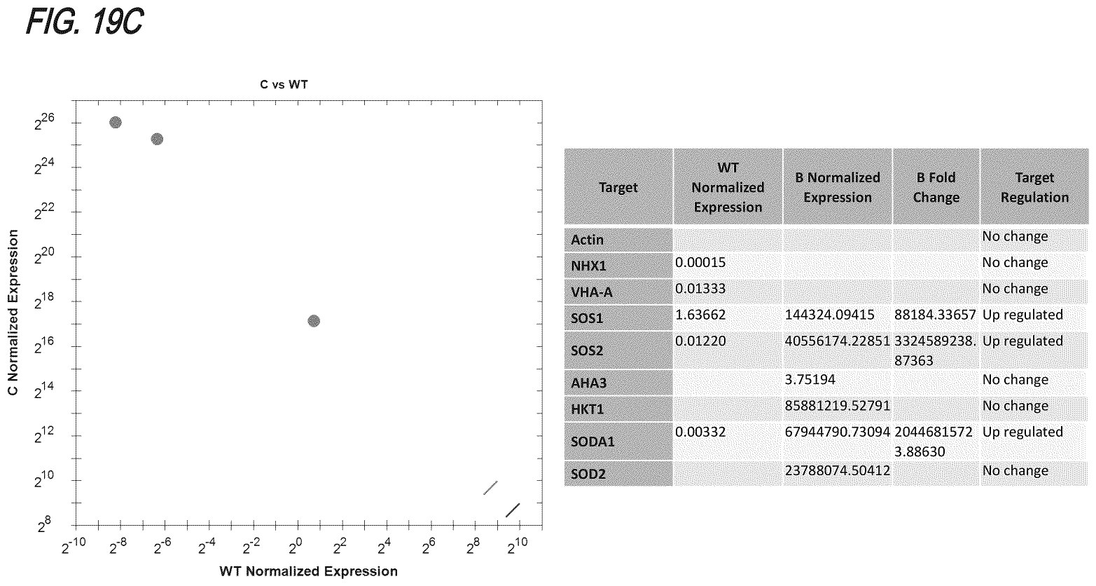

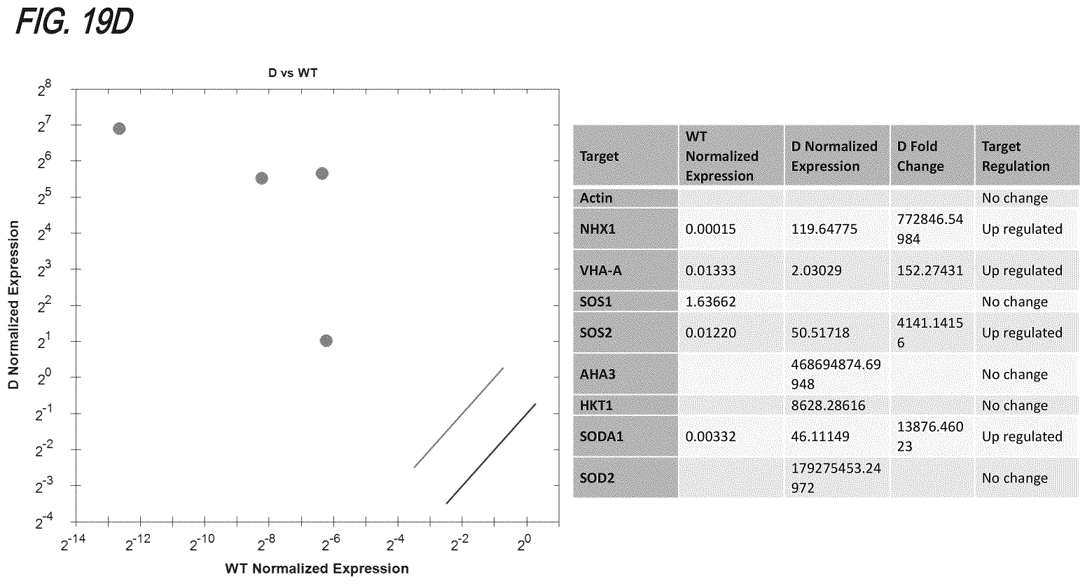

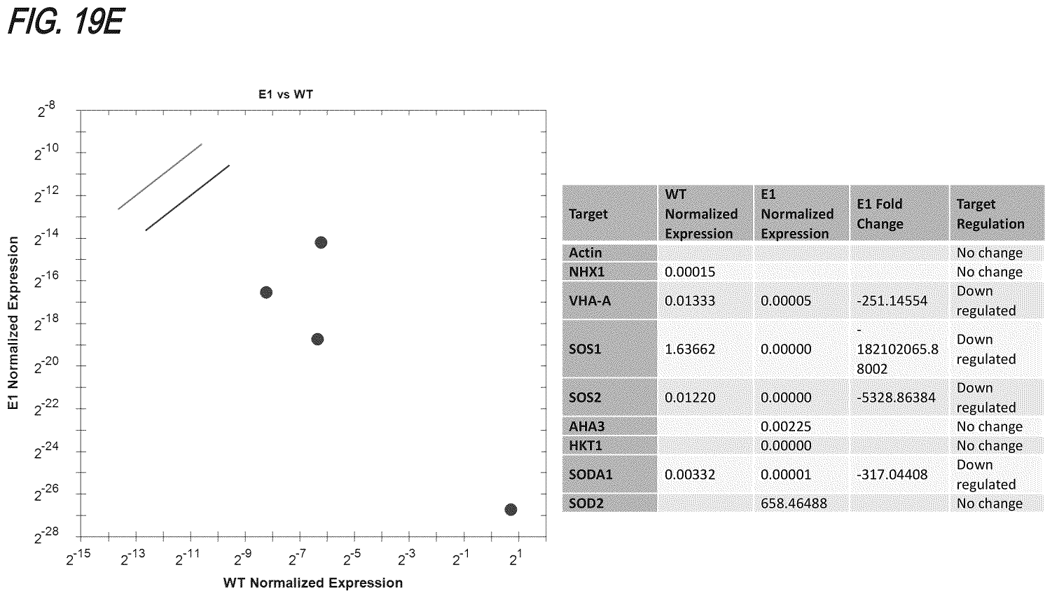

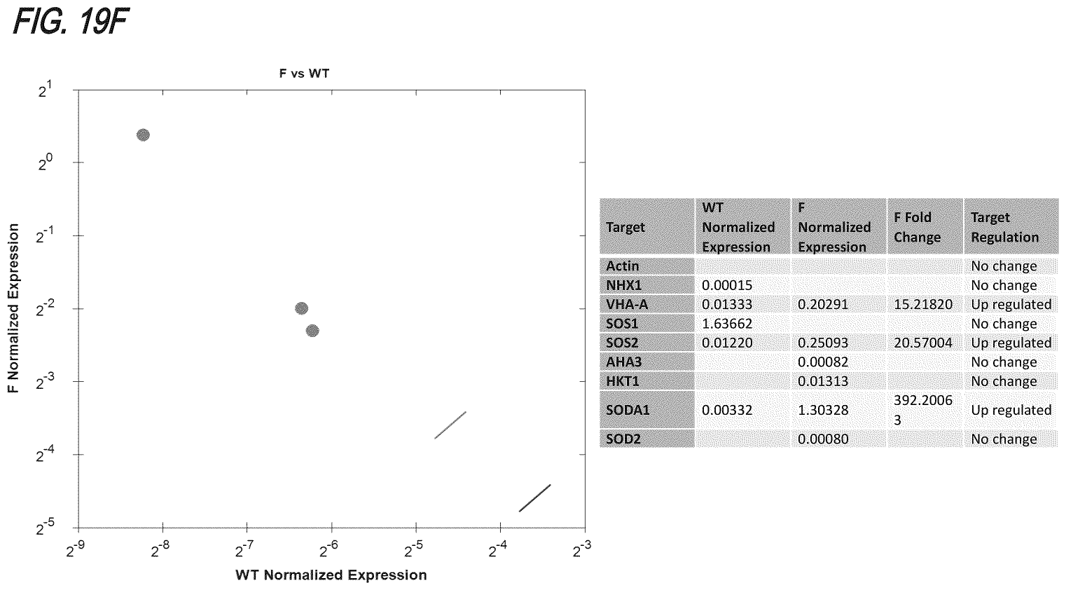

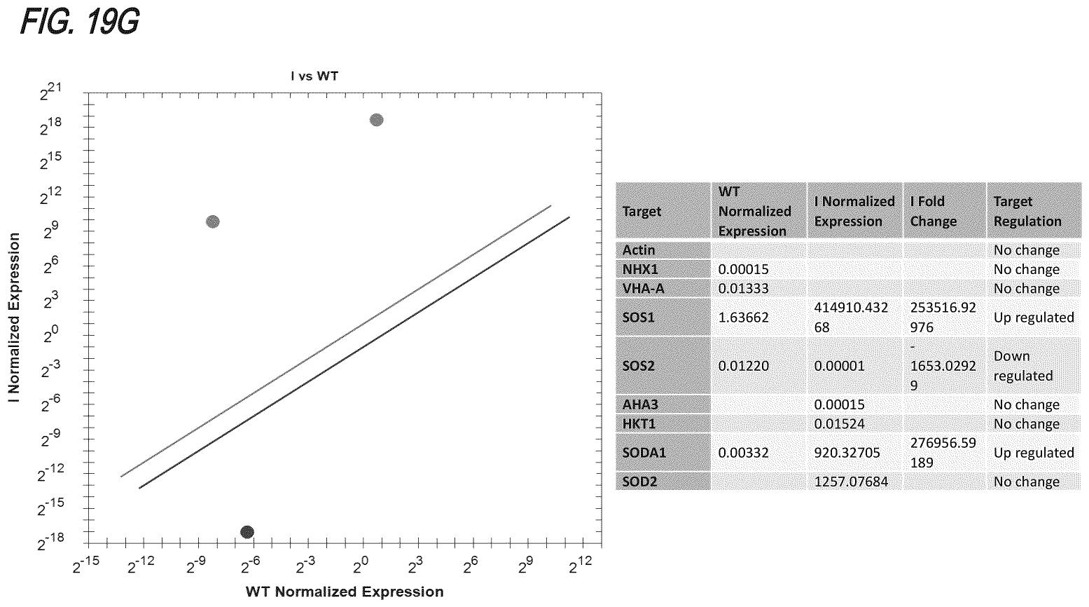

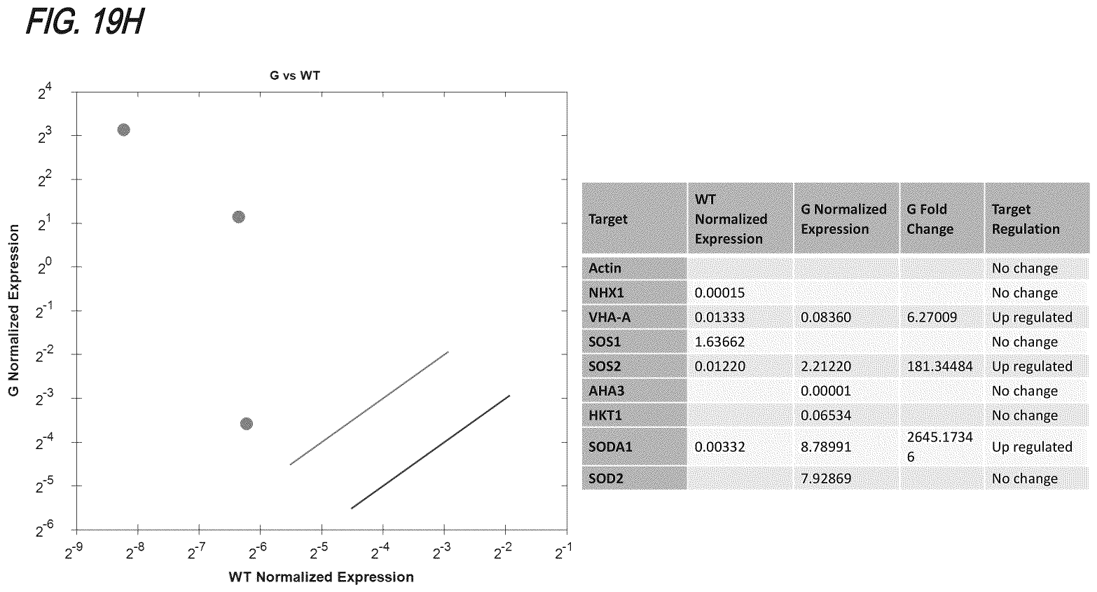

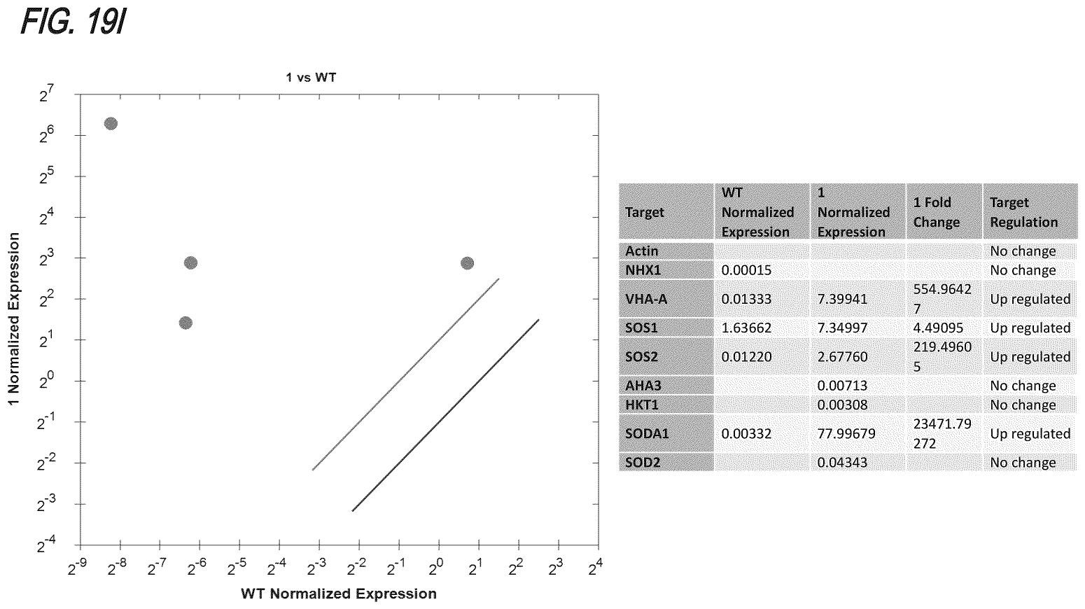

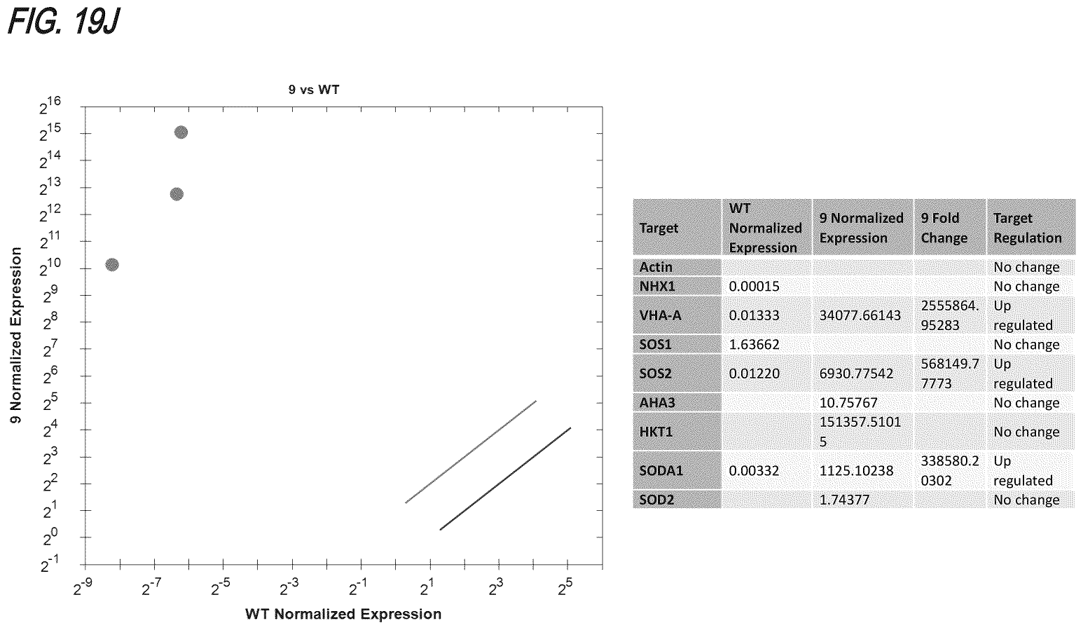

FIG. 18A, FIG. 18B, and FIG. 18C show the RNA expression levels of NHX1, SOS1, AHA3, HKT1, VHA-A, SOS2, SODA1, SOD2 and SODCC1 in tissue obtained from whole engineered plants. These data demonstrate that these genes were differentially expressed in the engineered plants compared to wild type plants. This difference is further evaluated in the scatter plots in FIG. 19A-FIG. 19J. The variation in the expression of these genes between the different engineered plants, seen in FIGS. 18A-18C and FIG. 19, may be due to differences in the types of tissue that were collected from each engineered plant. As mentioned previously, the expression of the genes of interest can vary spatially across the plant. For example, HKT1 is expressed only in the vasculature of the plant, therefore if the sample collected did not contain vasculature tissue then little to no HKT1 expression would be observed. SOD2 had reduced expression in a number of the engineered plants tested. A possible reason for this reduced expression may be that the wild type plant used as a control was a different age compared to the engineered plants.

### Example 5—Salinity Tolerance of the Engineered Plants

The salt tolerance of the engineered plants from Example 2 were tested by partially submerging the roots of the plantlets or a plant in Hydroponics Media initially with 0 g of salt.

Hydroponics Media

| 4.3 | g L−1 | Murashige & Skoog Basal Salts and Vitamins |
|---|---|---|
| 100 | mg L−1 | Myo-inositol |
| 60 | mg L−1 | Calcium Silicate |
| 50 mg L−1/90 mg L−1 | | Kanamycin/Cefotaxime (either antibiotic) |
| 20 | mg L−1 | Carbendazim (antifungal) |

The salinity of the hydroponics media was then increased incrementally up to 45 g L⁻¹, which is full oceanic salinity. The incremental changes in salt concentration and spacing between each change can be subject to change depending on performance of varieties. The growth rates of both shoots and roots was monitored and the overall health of the plant and colour of the leaves analysed.

| Day | NaCl gL−1 |
|---|---|
| 0 | 0 |
| 3 | 2 |
| 6 | 4 |
| 9 | 6 |
| 12 | 8 |
| 15 | 10 |
| 18 | 12 |
| 21 | 14 |
| 24 | 16 |
| 27 | 18 |
| 30 | 20 |
| 33 | 23 |
| 36 | 26 |
| 39 | 29 |
| 42 | 32 |
| 45 | 35 |

### Example 6—Salinity Tolerance of the Engineered Plants

The salt tolerance of the engineered plants from Example 2 were tested by growing the plants in media that had been supplemented with sodium chloride. Engineered plants A, B, C, D, E1, F, G, H, & I from Example 2 were placed directly into RRM II Media supplemented with an initial 8g/I NaCl and later an increased 16 g/L NaCl. The engineered plants continued to grow under these high salinity conditions. Wild-type plants were initially placed in MS media supplemented with 2 g/L NaCl. The salinity was increased by 2 g/L every two days until 10 g/L NaCl.

MS Media

| | | |
|---|---|---|
| 4.3 | g L⁻¹ | Murashige & Skoog Basal Salts and Vitamins |
| 2, 4, 6, 8, 10 | g L⁻¹ | Maltose |
| 50 | mg L⁻¹ | Vancomycin/Cefotaxime (either antibiotic) |
| 20 | mg L⁻¹ | Carbendazim (antifungal) |

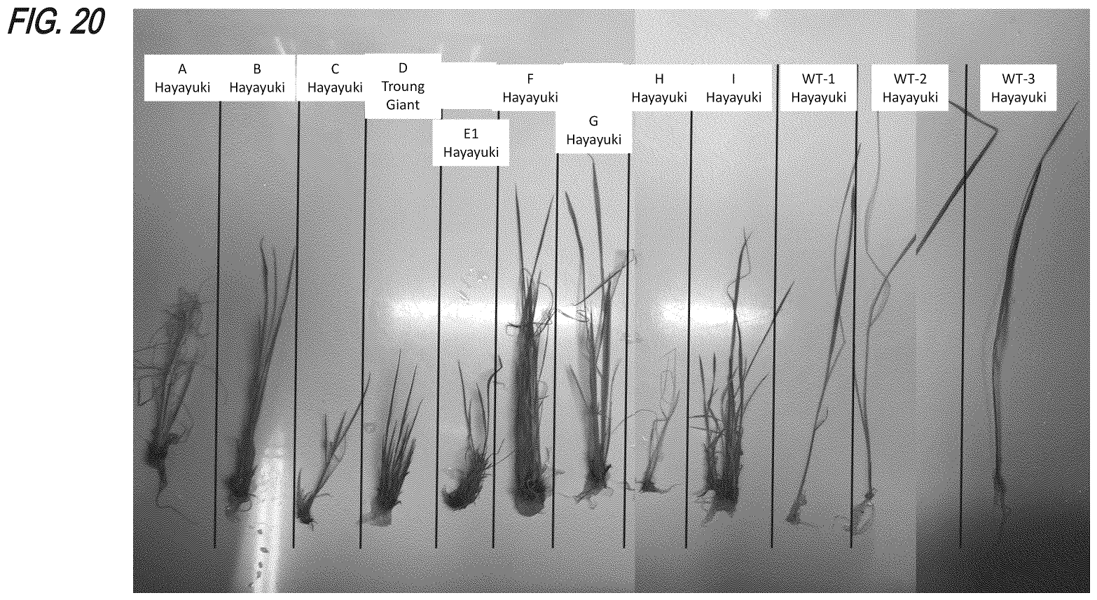

FIG. 20 provides images of the engineered and wild-type plants on the final day of the experiment. The images in FIG. 20 show that the wild-type plants all exhibit significant yellowing of at least half the leaves. Starting at the leaf tip and often spreading throughout the entire length of the leaf. This yellowing is not observed in the engineered plants. The insertion of the enhancer elements so that they are operatively linked to the genes of interested has enabled the engineered plants to tolerate much higher concentrations of salt without showing physical signs of stress. The data demonstrate that the engineered plants described herein have increased salt tolerance compared to wild-type plants.

Some of the engineered plants appear white (A & H). These plants have exhibited this colour throughout their entire post regenerated life cycle. Without wishing to by bound to any particular theory, the inventors hypothesize that is due to the substantial increase in gene expression causing a reduction in chlorophyll production. These plants have still behaved similarly to their green counterparts.

The wild-type controls show significantly reduced root and leaf quantity. Their structure is also thinner and unable to support the weight of the plant unaided. These characteristics show that the increased salinity has affected the health of the wild-type plants. In contrast the engineered plants do not exhibit these characteristics further suggesting that the edits that have been made to the genome not only allow the plants to survive, but also to thrive in highly saline environments that wild-type rice are unable to grow in. This improvement is seen in multiple rice varieties demonstrating that inventors designed can be applied to various rice varieties.

Overall, these data demonstrate the plants that have been engineered to have enhancer elements operatively linked to particular genes of interest can grow at high salinities and thus have improved salt tolerance compared to wild-type plants.

### Example 7—Improved Design of Genetic Inserts

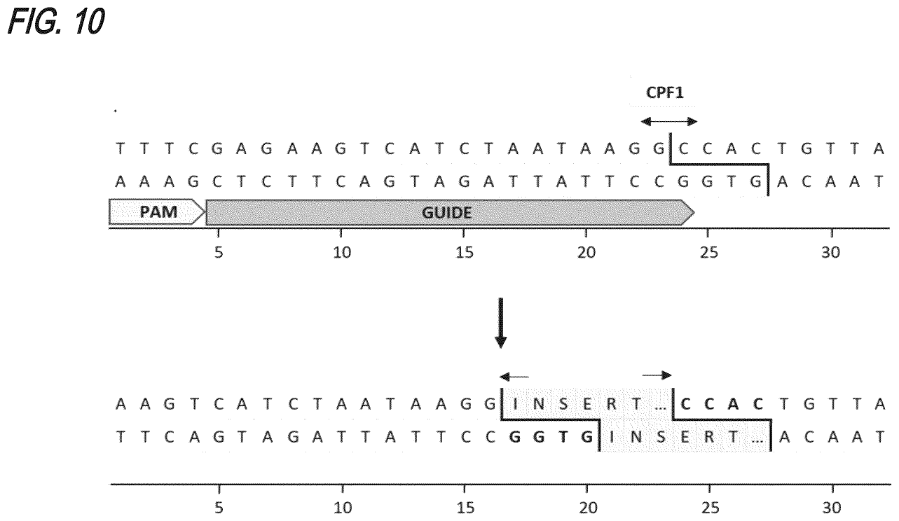

The design of the genetic inserts was altered to improve the editing efficiency and minimize the amount of DNA required to make a successful edit. The main focus for this improved design was to use Cpf1 (Cas12a) to make incisions within the genomic sequence where Cas9 was previously used. Cas9 makes blunt-ended cuts that require large homology arms (˜125 bases including the genetic inserts) to successfully edit and insert any amount of DNA into a genomic sequence, this entire section of DNA is incorporated into the plants resulting in a ˜120 base pair edit per gene. In comparison, Cpf1 uses staggered cuts, making incisions 19 base pairs after the PAM site on the sense strand and 23 base pairs after the PAM site on the anti-sense strand, as shown in FIG. 10. Cpf1 edits do not require homology arms and instead rely on 4-5 base pair overhangs that can be seen in bold in FIG. 10.

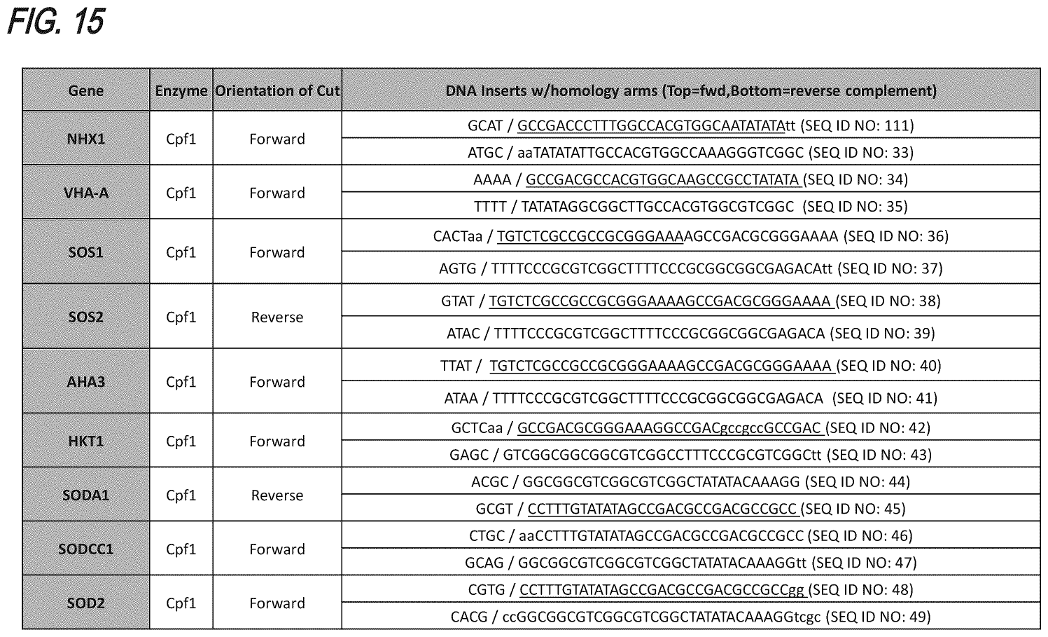

These overhangs were included at the ends of the genetic inserts to enable a smaller piece of DNA to be integrated into the genome using the sequences in FIG. 15. The edit sites were designed to ensure normal function of the gene is left unchanged, only changing the expression intensity and expression location pattern. In this updated design, the gene of interest VHA-A, SOS2, AHA3, and SODA1 have all been converted to accommodate Cpf1 instead of Cas9. The updated guide RNA (gRNA) and updated forward and reverse insert sequences with relevant overhang sequences are listed in Table 12 and FIG. 15.

In combination with Example 1, these data demonstrate that the enhancer elements can be integrated into the genome of the plant using a variety of nucleases. Cpf1 nuclease may be used to integrate the enhancer elements into the genome because it results in a more efficient method of generating the engineered plants.

TABLE 12
The guide RNAs for Cpf1 nucleases

| Gene | Guide RNA sequence |
|---|---|
| NHX1 | TTTACCGGGAATTGTCGAATTAGGC (SEQ ID NO: 115) |
| VHA-A | TTTAAAATTCGCTGGAGGAAAAAAA (SEQ ID NO: 116) |
| SOS1 | TTTGCCTTGGATTTTCCACTTGCA (SEQ ID NO: 117) |
| SOS2 | TTTGGGGGGGCTTCGCTGCCTGAAT (SEQ ID NO: 118) |
| AHA3 | TTTGCAAGCAAGATTTCCTTTGCTT (SEQ ID NO: 119) |
| HKT1 | TTTCTCATACTCGTTGGCTCGTTGC (SEQ ID NO: 120) |
| SODA1 | TTTATGCGGCATCTGTCTCGTGAAC (SEQ ID NO: 121) |
| SODCC1 | TTTAGGACCTCTAGAGGCTACTTCT (SEQ ID NO: 122) |
| SOD2 | TTTGGTGGACTACTCGATCTCTGCG (SEQ ID NO: 123) |

### Example 8—Production of Engineered Calli

Calli were produced as described in Example 3. These calli were bombarded with the DNA inserts described in Example 7 which use Cpf1 nucleases only to make incisions within the genomic sequence. The DNA inserts targeted four genes of interest—OsNHX1, OsVHA-A, OsSOS1 & OsSOS2 and their sequences are provided in FIG. 15. The OsSOS1 gene is operably linked to an enhancer element comprising SEQ ID NO: 12, the OsSOS2 gene is operably linked to an enhancer element comprising SEQ ID NO: 13, the OsVHA-A gene is operably linked to an enhancer element comprising SEQ ID NO: 11 and the OsNHX1 gene is operably linked to an enhancer element comprising SEQ ID NO: 10.

These four genes of interest were chosen as they are most likely be expressed within calli, considering the difference in physiology, hormone and transcription factor abundance compared to a regenerated engineered plant. This set of genes of interest was also chosen because they act synergistically to drive salt exclusion and isolation within any plant cell to drive salt tolerance.

### Example 9—Salinity Tolerance of the Calli

The salt tolerance of the engineered calli from Example 8 were tested by transferring the calli to Rice Callus Induction Media supplemented with 2 gL⁻¹ of sodium chloride. Every two days the calli were transferred to a higher concentration of salt. The concentrations tested are shown below:

| Day | NaCl gL⁻¹ |
|---|---|
| 0 | 2 |
| 2 | 4 |
| 4 | 6 |
| 6 | 8 |
| 8 | 10 |
| 10 | 12.5 |
| 12 | 15 |

The maximum of 15 gL⁻¹ sodium chloride is just below 50% of full ocean salinity, because the calli are not expected to perform optimally compared to a whole plant. However, calli that can grow in 15 gL⁻¹ will have a significantly high salt tolerance, well beyond the tolerance of the rice varieties currently in use commercially and beyond the salinity of paddies in the Mekong (for example). Overall callus health will also be recorded including: Growth rates; Colour; Texture and Structural integrity.

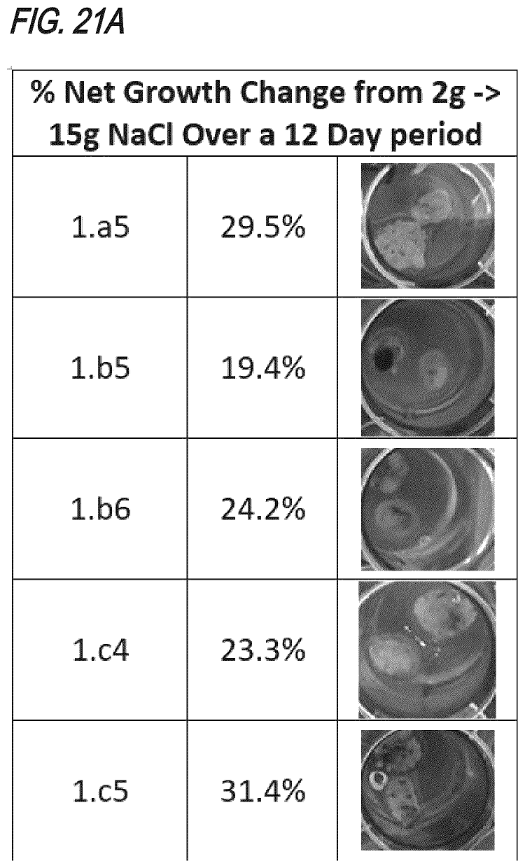

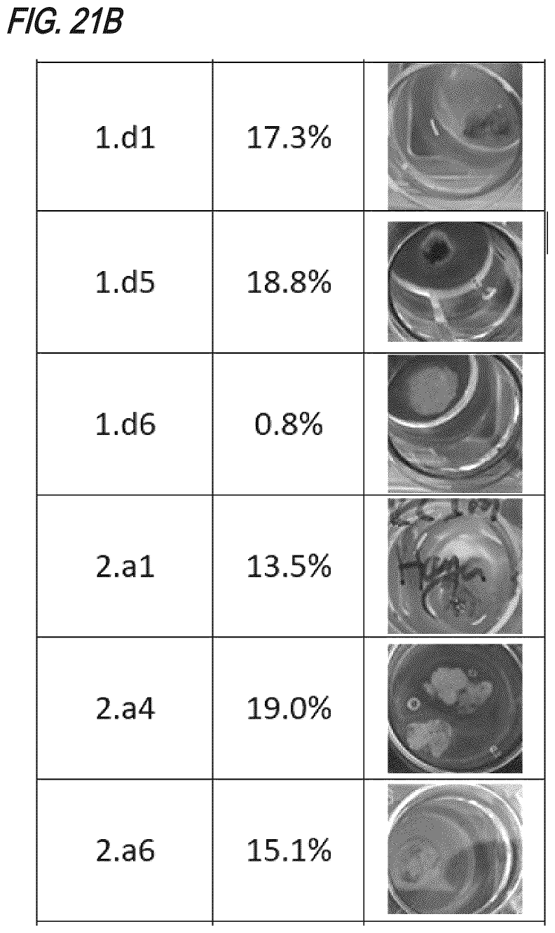

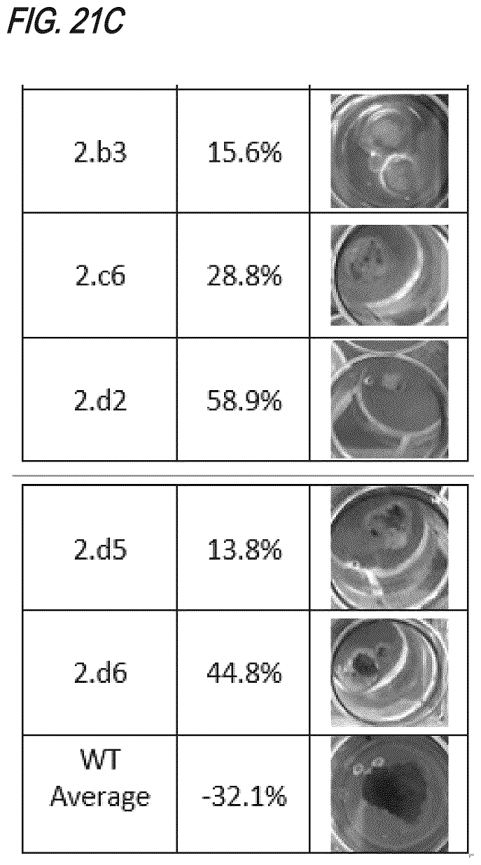

FIG. 21A-FIG. 21C demonstrate that the engineered calli were able to grow in media that contained up to 15 gL⁻¹ of sodium chloride, whereas wild-type calli were not able to grow under the same conditions. Identifiers of callus health included colour (white/yellowish as opposed to black) and texture (friable and not mushy). The images in FIG. 21A-FIG. 21C demonstrate that in general the engineered calli appeared whiter with more defined edges than the WT, which indicates that these calli had higher tolerance.

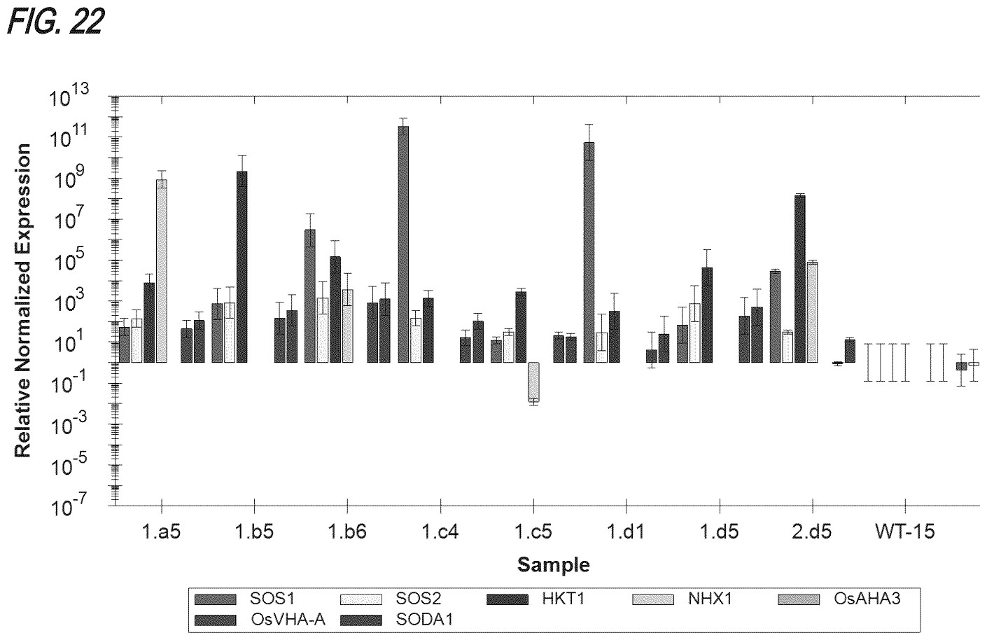

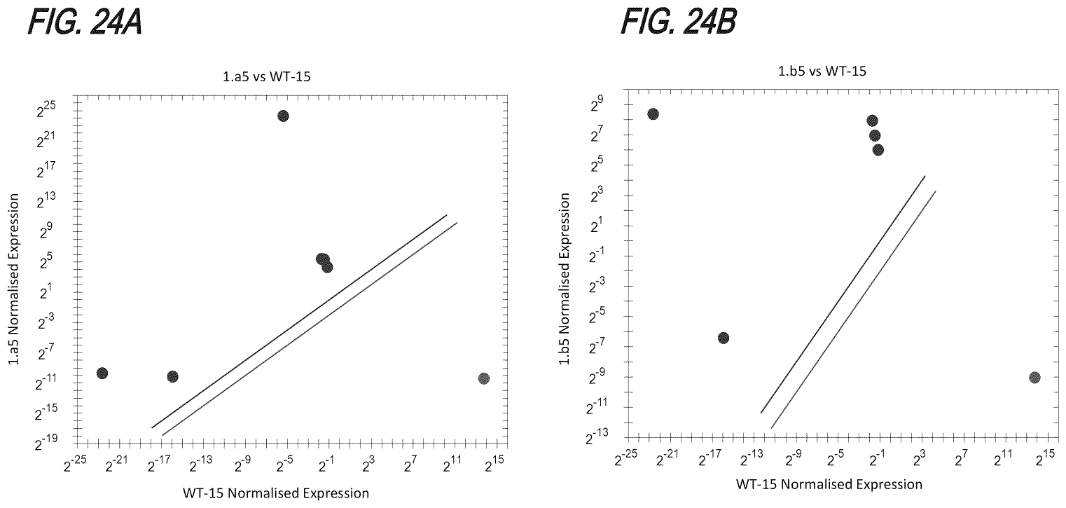

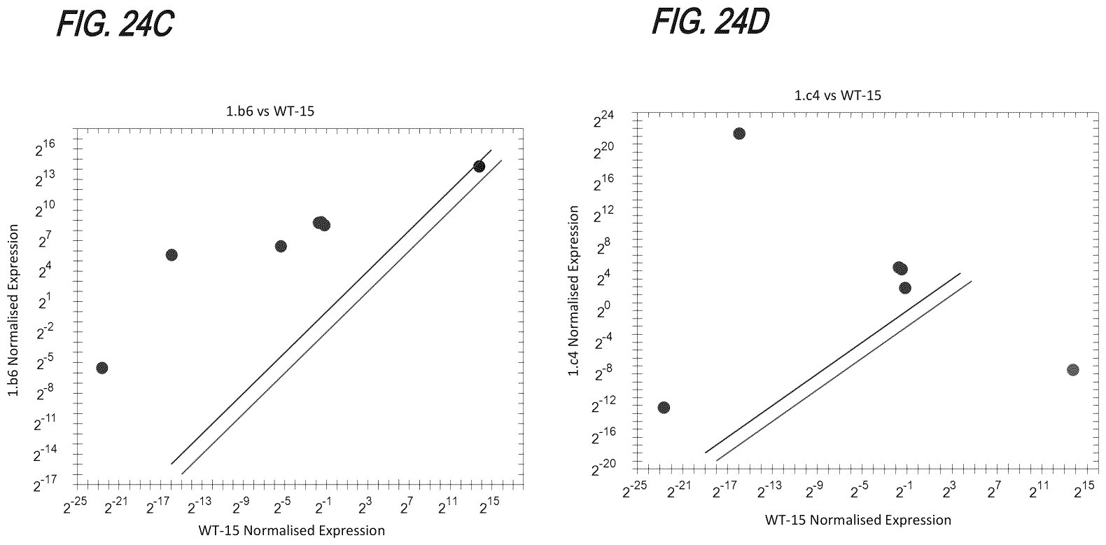

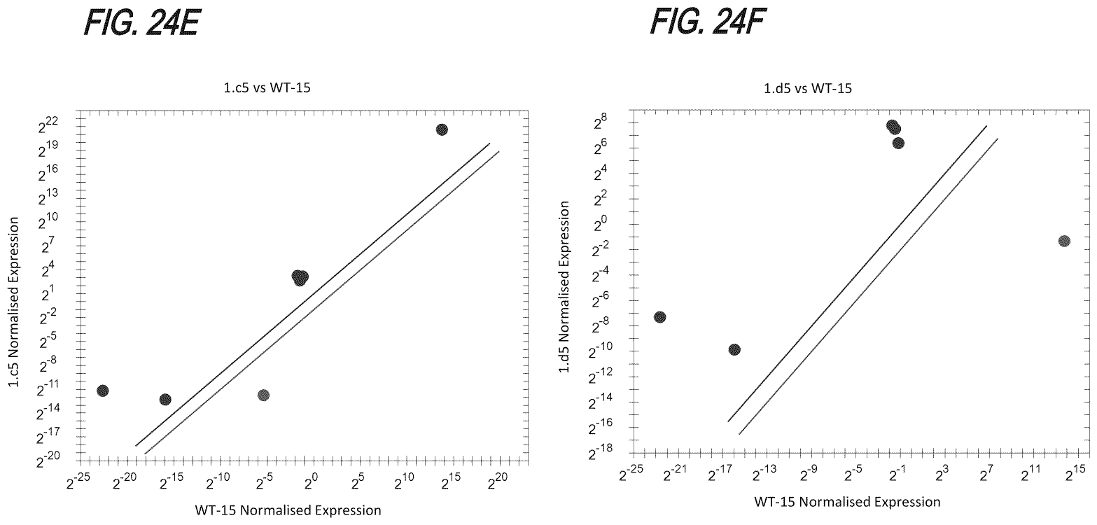

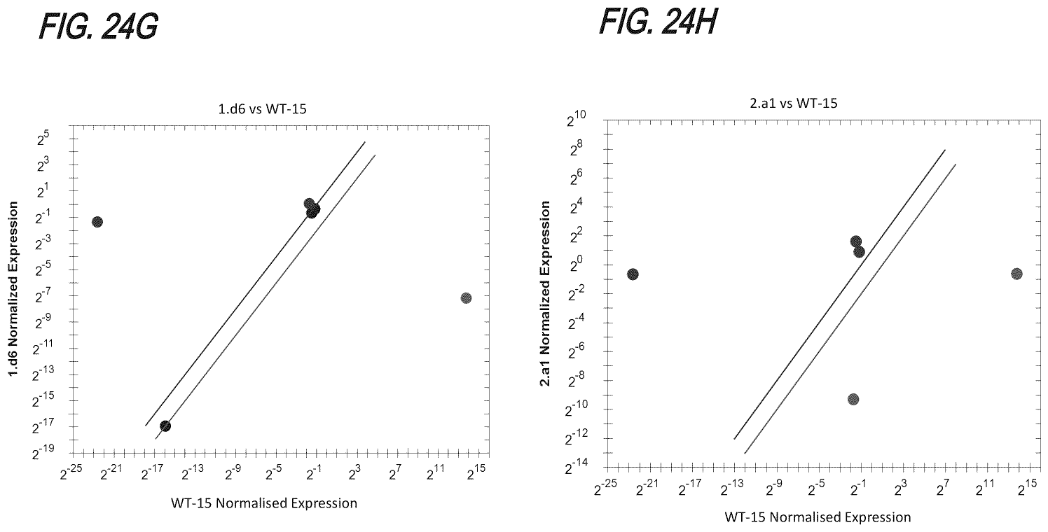

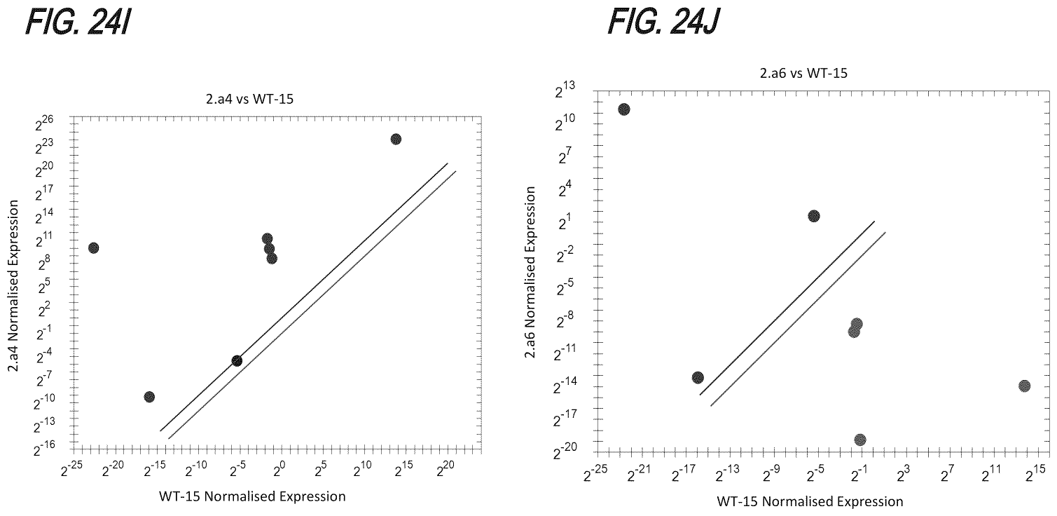

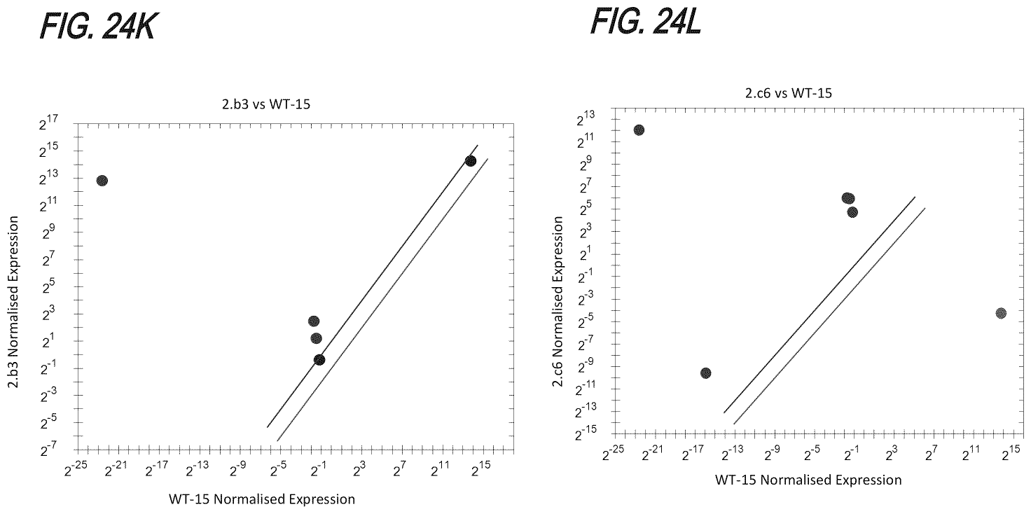

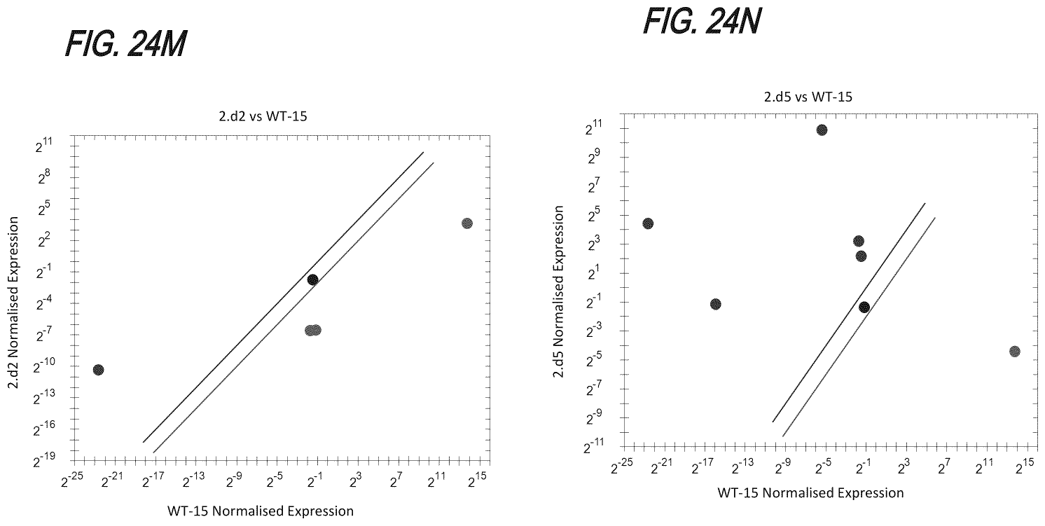

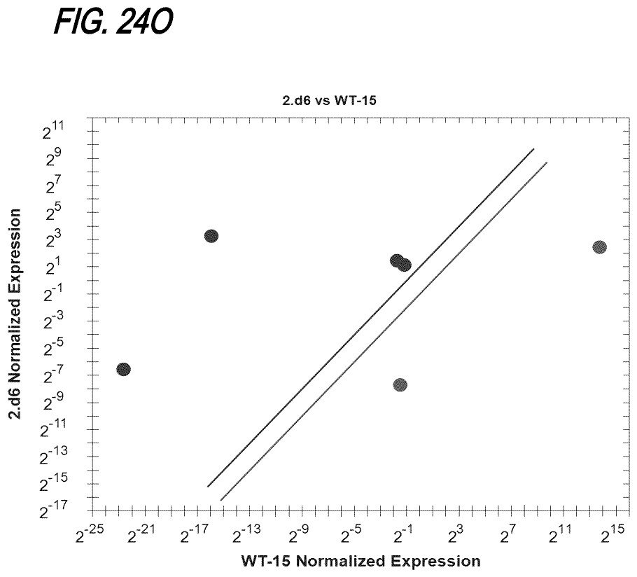

q-RT-PCR was performed on the engineered calli as described in Example 4. FIG. 22 shows the gene expression of SOS1, SOS2, HKT1, NHX1, AHA-3, VHA-A, and SODA1 in the engineered calli. FIG. 22 demonstrates that the majority of engineered calli had increased SOS1, SOS2, HKT1, NHX1, AHA-3, VHA-A, and SODA1 expression compared to wild-type calli when grown at 15 g/L of sodium chloride. This difference is further evaluated in the scatter plots in FIG. 24A-FIG. 24O. These data demonstrates that the bombardment process has successful integrated enhancer elements into the genome of the plant so that they are operatively linked to the genes of interest, which enabled the engineered calli to grow under increased saline stress compared to the wild-type.

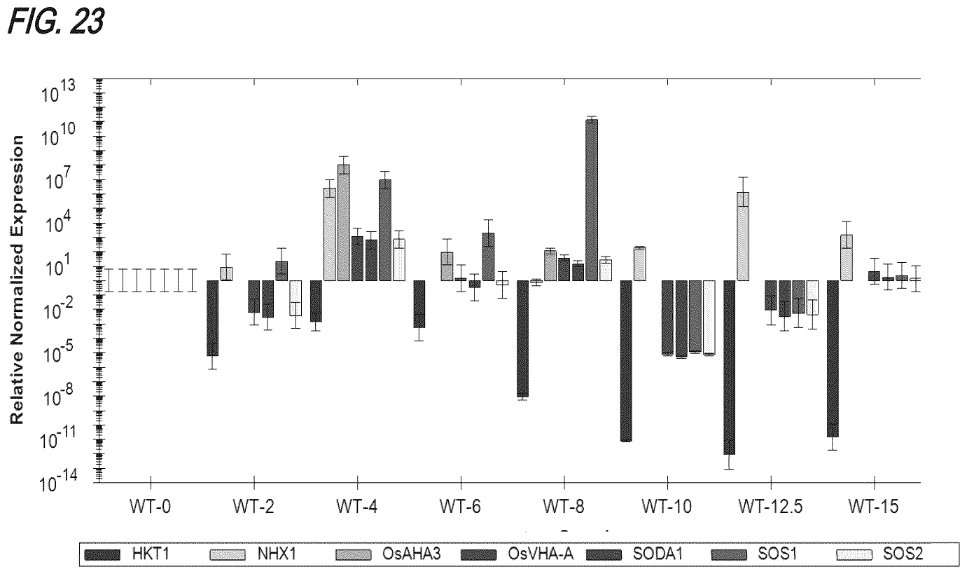

In previous studies it was observed that rice calli show a sharp decrease in fresh weight at 8.7 g/L NaCl (Wani et al 2010 The Asian and Australasian Journal of Plant Science and Biotechnology, 4(1), pp. 57-61), as our transformed calli exhibit continued growth through to 15 g/L NaCl we have observed a huge increase in callus salinity tolerance. qRT PCR analysis was conducted on the wild-type calli taken at 0, 2, 4, 6, 8, 10, 12.5 & 15 g/L NaCl. As seen in FIG. 23, in the interval between 8 g/L and 10 g/L there is a drastic change in the expression of NHX1, OsAHA3, OsVHA-A, SODA1, SOS1 and SOS2, indicating a similar shift where calli become unable to tolerate such high concentrations of NaCl. Overall, the data is similar to the physical characteristics observed in the literature of calli grown in salt.

### Example 10—Production of Genetic Inserts for Engineered Kale (Brassica oleracea)

Genetic inserts were designed to stably introduce enhancer elements into the genome of kale using Cpf1 nucleases. These genes of interest encode a variety of proteins with different functions including a plasma membrane ion transporter; a plasma membrane hydrogen exporting ATPase, a tonoplast hydrogen exporting ATPase, a tonoplast ion transporter, a potassium transporter, a protein kinase and an antioxidant.

TABLE 13
The sequences added to the 5′ and 3′ ends of the DNA inserts for engineered Kale.
Underlining signifies adapter sequences cleaved by restriction enzymes

| Rice | Kale | DNA | cpf1 DNA |
|---|---|---|---|
| gene | orthologue | insert forward primer | insert reverse primer |
| NHX1 | Bo9g010200 | ATAGCACAATGCCGACCCTTT | CTATTATATATTGCCACGTGGCC |
| | | GGCCACGTGGCAATATATA | AAAGGGTCGGCATTGTG (SEQ ID |
| | | (SEQ ID NO: 59) | NO: 60) |
| VHA-A | Bo6g122400 | TCGGCACAATGCCGACGCCAC | CCGATATATAGGCGGCTTGCCAC |
| | | GTGGCAAGCCGCCTATATA | GTGGCGTCGGCATTGTG (SEQ ID |
| | | (SEQ ID NO: 61) | NO: 62) |
| SOS1 | Bo9g071470 | CTCTTGACGCACAATCCCACTT | AGAGTTATATATTTTCCCGCGTC |
| | | GTCTCTATATAGCCGCCGCGG | GGCTTTTCCCGCGGCGGCTATAT |
| | | GAAAAGCCGACGCGGGAAAAT | AGAGACAAGTGGGATTGTG(SEQ |
| | | ATATAA(SEQ ID NO: 63) | ID NO: 64) |
| SOS2 | Bo4g139520.1 | GTAACTGACGCACAATCCCAC | TTACGCTTATATATTTTCCCGCGT |
| | | TTGTCTCTATATAGCCGCCGC | CGGCTTTTCCCGCGGCGGCTATA |
| | | GGGAAAAGCCGACGCGGGAA | TAGAGACAAGTGGGATTGTGCGT |
| | | AATATATAAGC(SEQ ID NO: 65) | CAG (SEQ ID NO: 66) |
| OsAHA3 | Bo9g133110 | CATCCACAATCCCACTACCTC | GATGTATATATTTTCCCGCGTCG |
| | | GTGTCTCGCCGCCGCGGGAA | GCTTTTCCCGCGGCGGCGAGAC |
| | | AAGCCGACGCGGGAAAATATA | ACGAGGTAGTGGGATTGTG (SEQ |
| | | TA (SEQ ID NO: 67) | ID NO: 68) |
| HKT1 | Bo3g044660 | ACACCACAATACCTCGTGTCT | GTGTGTCGGCGGCGGCGTCGGC |
| | | CGCCGACGCGGGAAAGGCCG | CTTTCCCGCGTCGGCGAGACAC |
| | | ACGCCGCCGCCGAC (SEQ ID | GAGGTATTGTG |
| | | NO: 69) | (SEQ ID NO: 70) |
| [CuZn]SOD2 | Bo5g137020 | ATGACACAATCCCACTCCTTTG | TCATGAGACACGAGGTGGCGGC |
| | | TATATAGCCGACGCCGACGCC | GTCGGCGTCGGCTATATACAAAG |
| | | GCCACCTCGTGTCTC (SEQ ID | GAGTGGGATTGTG |
| | | NO: 71) | (SEQ ID NO: 72) |
| SODA1 | Bo1g141360 | TCTCCACAATCCCACTCCTTTG | GAGAGAGACACGAGGTGGCGGC |
| | | TATATAGCCGACGCCGACGCC | GTCGGCGTCGGCTATATACAAAG |
| | | GCCACCTCGTGTCTC (SEQ ID | GAGTGGGATTGTG |
| | | NO: 73) | (SEQ ID NO: 74) |
| CuZn SOD | Bo4g165150 | ACGCCACAATCCCACTCCTTT | GCGTGAGACACGAGGTGGCGGC |
| chloroplast | | GTATATAGCCGACGCCGACGC | GTCGGCGTCGGCTATATACAAAG |
| | | CGCCACCTCGTGTCTC (SEQ | GAGTGGGATTGTG |
| | | ID NO: 75) | (SEQ ID NO: 76) |
| OSK1 | Bo4g138000 | TTTCCCTCGTGTCTCATGACG | GAAATATATACGGCGGCGTCGGC |
| | | CACAATCCCACTCCTTTGTATA | GTCGGCTATATACAAAGGAGTGG |
| | | TAGCCGACGCCGACGCCGCC | GATTGTGCGTCATGAGACACGAG |
| | | GTATATA (SEQ ID NO: 77) | G (SEQ ID NO: 78) |

### Example 11—Genomic Sequencing of the Engineered Plants

The genomic DNA of the engineered plants produced in Example 2 were sequenced to determine whether the genes of interest had been introduced successfully into the plant after bombardment.

The genomic DNA was isolated and purified using a Wizard® Genomic DNA Purification Kit. Briefly, 40 mg of lead material was harvested from the target plant. The leaf tissue was frozen in liquid nitrogen and ground into a fine powder using a pestle and mortar. 600 μl of Nuclei Lysis Solution was added to the sample, which was then vortexed for 1-3 seconds. The sample was then incubated at 65° C. for 15 minutes. 3 μl of RNase Solution was added to the cell lysate and mixed by inverting the tube 2-5 times. The mixture was incubated at 37° C. for 15 minutes. The sample was cooled at room temperature for 5 minutes and then 200 μl of Protein Precipitation Solution was added. The sample was vortexed vigorously at high speed for 20 seconds and then centrifuged for 3 minutes at 13,000-16,000×g. The precipitated proteins formed a tight pellet. The supernatant was transferred to a tube containing 600 μl of room temperature isopropanol. The sample was mixed by inverting the tube and then centrifuged at 13,000-16,000×g for 1 minute at room temperature. 600p1 of room temperature 70% ethanol was added and the tube gently inverted several times to wash the DNA. The sample was then centrifuged at 13,000-16,000×g for 1 minute at room temperature. The ethanol was removed the pellet containing the DNA was air-dried for 15 minutes. 100 μl of DNA Rehydration Solution was added and the sample incubated at 65° C. for 1 hour. The concentration of each DNA sample was measured using a Nanodrop spectrophotometer. The resulting DNA was sequenced by Eurofins Genomics lab.

### Example 12—Stable Inheritance of Salt Tolerance in Engineering Plants

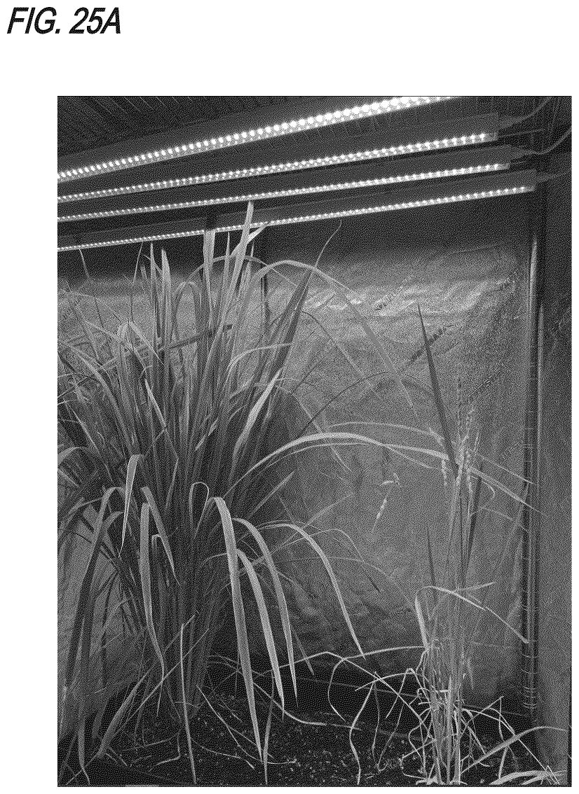

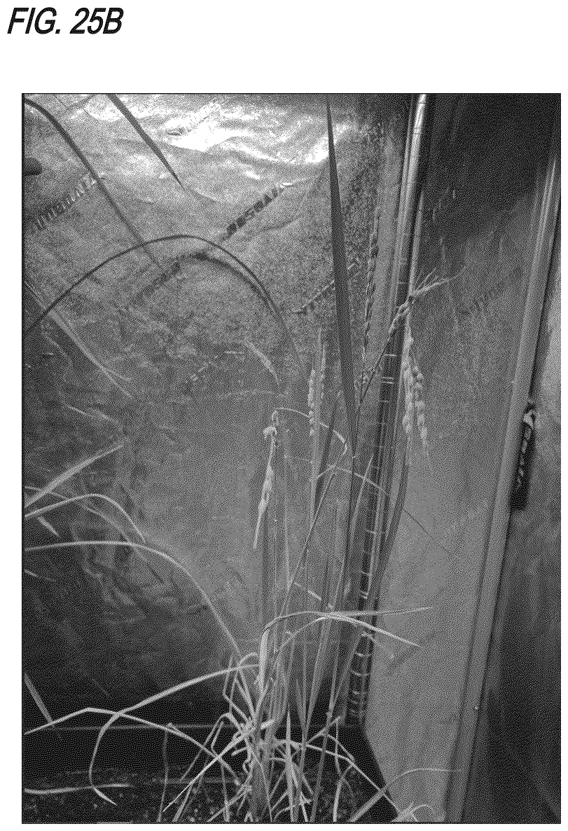

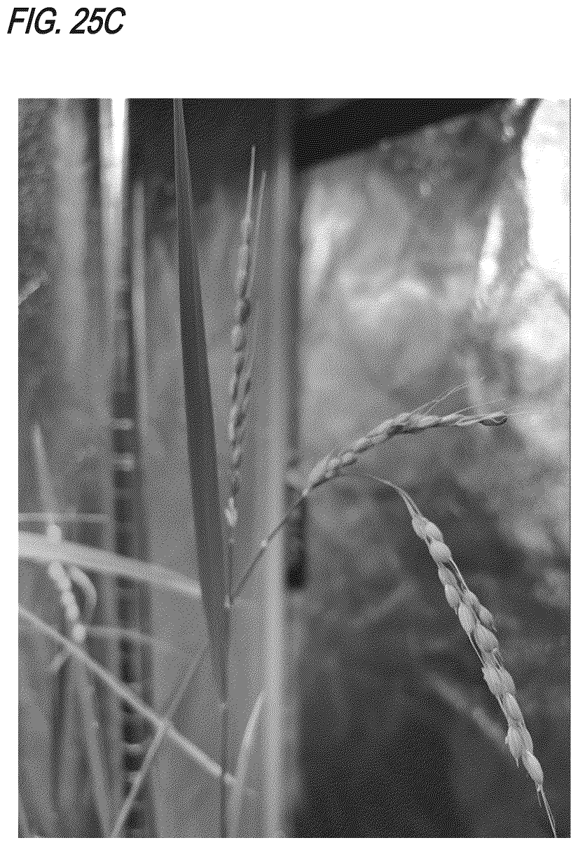

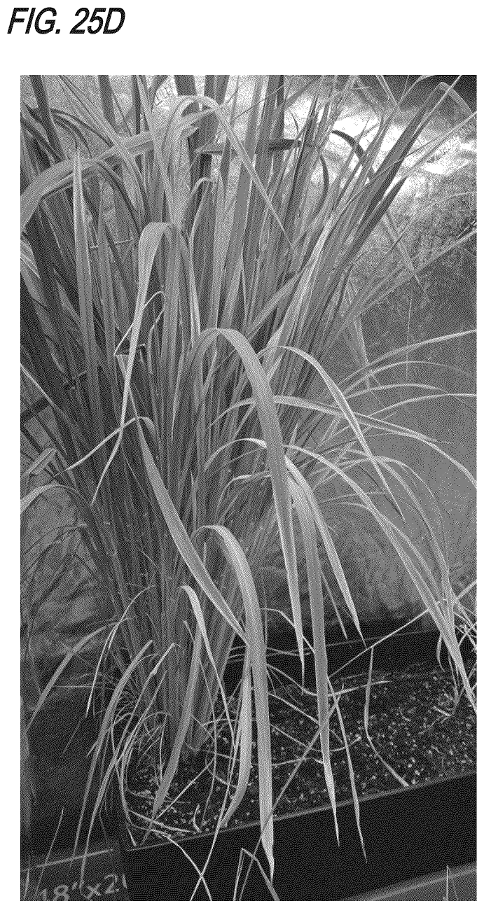

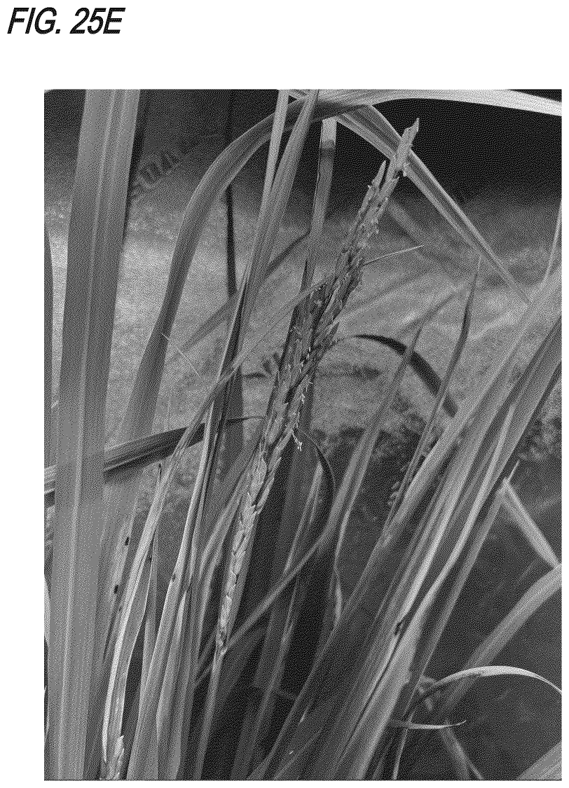

Engineered rice plants of the Hayayuki and Truong Giang varieties were produced as described in Example 2. These engineered rice plants comprised the following genes of interest, OsNHX1, OsVHA-A, OsSOS1, OsSOS2, OsAHA3, OsHKT1, and either OsSODA1 or OsSOD2, wherein each gene of interest is operatively linked to an enhancer element. These engineered rice plants were grown to maturity so that they produced seeds. FIG. 25A-FIG. 25E are images of these engineered rice plants showing the panicles and seeds.

Seeds were harvested from these three plants (Hayayuki 1, Hayayuki 2 and Truong Giang) and germinated to seedlings. The gene expression in the leaves of the progeny plants was analyzed using qRT-PCR as performed in Example 4 to observe whether salt tolerance had been successfully inherited.

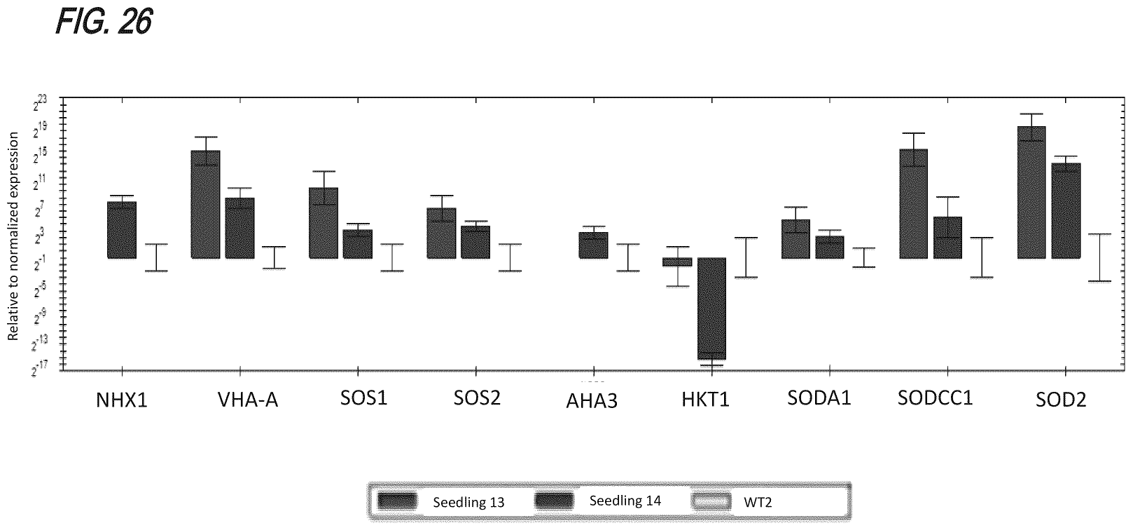

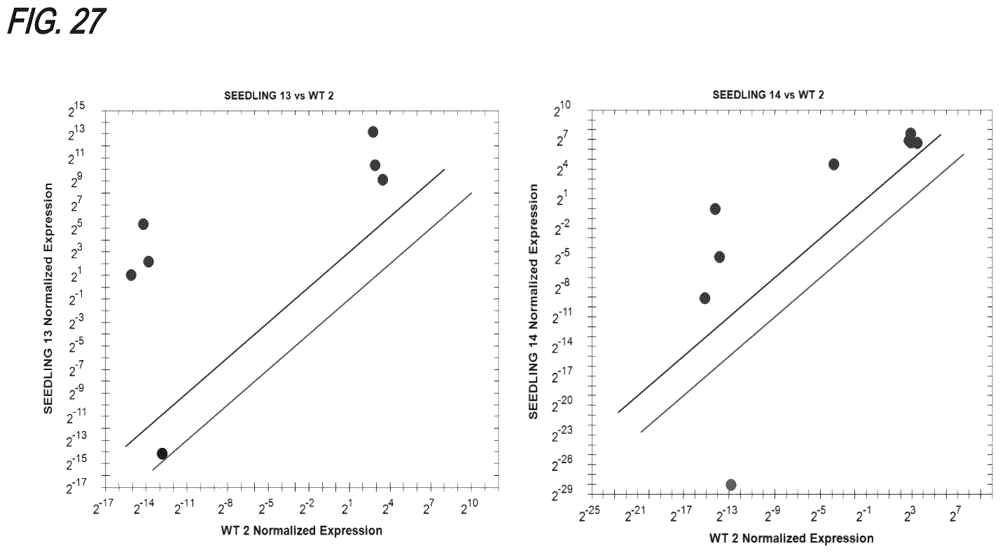

Seedlings 13 and 14 are two seedlings that were grown from seeds collected from Hayayuki Plant 1. FIG. 26 shows that the RNA expression levels of OsNHX1, OsVHA-A, OsSOS1, OsSOS2, OsAHA3, OsSODA1, SODCC1 and OsSOD2 in these seedlings was significantly higher than compared to the wild type rice. These data demonstrate that these genes were differentially expressed in the progeny compared to wild type plants. This difference is further evaluated in the scatter plots provided in FIG. 27. The gene expression observed in the two seedlings is similar to the gene expression observed in the parent plant (Hayayuki 1) and to other engineered rice plants described herein. These data demonstrate that the salt-tolerance traits transformed into the engineered plants described herein are successfully inherited.

Additional sequences

SEQ ID NO: 92-OsSODA1-Q43121
MALRTLASRKTLAAAALPLAAAAAARGVTTVALPDLPYDYGALEPAISGEIMRLHHQKHHATYVANYNKALEQLDAA
VAKGDAPAIVHLQSAIKFNGGGHVNHSIFWNNLKPISEGGGDPPHAKLGWAIDEDFGSFEALVKKMSAEGAALQGSG
WVWLALDKEAKKLSVETTANQDPLVTKGANLVPLLGIDVWEHAYYLQYKNVRPDYLSNIWKVMNWKYAGEVYENATA
SEQ ID NO: 93-OsSOD2-Q10PW4
MAGKAGGLKGVALIGGAGGNSAVAGALHFFQDPSTGYTEVRGRVTGLAPGLHGFHIHSFGDTTNGCNSTGPHENPHN
KSHGAPSDDERHVGDLGNIVANKDGVADIFIKDLQISLSGPHSILGRAVVVHADSDDLGRGGHELSKTTGNAGARIG
CGIIGLRSAV
SEQ ID NO: 94-OsSODCC1-Q0DRV6
MVKAVVVLGSSEIVKGTIHFVQEGDGPTTVTGSVSGLKPGLHGFHIHALGDTTNGCMSTGPHYNPAGKEHGAPEDET
RHAGDLGNVTAGEDGVANIHVVDSQIPLTGPNSIIGRAVVVHADPDDLGKGGHELSKTTGNAGGRVACGIIGLQG
SEQ ID NO: 95-OsSOS1-Q5ICN3
MDNPEAEPDDAVLFVGVSLVLGIASRHLLRGTRVPYTVALLVLGVALGSLEFGTKHGMGKLGAGIRIWANINPDLLL
AVFLPALLFESSFSMEIHQIKKCMAQMVLLAGPGVLISTFFLGSALKLTFPYNWNWKTSLLLGGLLSATDPVAVVAL
LKELGASKKLSTIIEGESLMNDGTAIVVYQLFYRMVLGRTFDAGSIIKFLSEVSLGAVALGLAFGIASVLWLGFIEN
DTIIEIALTLAVSYIAFFTAQDALEVSGVLTVMTLGMFYAAFAKTAFKGDSQQSLHHFWEMVAYIANTLIFILSGVV
IADGVLENNVHFERHGASWGFLLLLYVFVQISRILVVVILYPLLRHFGYGLDLKEATILVWAGLRGAVALSLSLSVK
RASDAVQTHLKPVDGTMFVFFTGGIVELTLIFNGSTTQFLLHLLGMDRLAATKLRILNYTKYEMLNKALEAFGDLRD
DEELGPPADWVTVKKYITCLNDLDDEPVHPHAVSDRNDRMHTMNLRDIRVRLLNGVQAAYWGMLEEGRITQTTANIL
MRSVDEAMDLVPTQELCDWKGLRSNVHFPNYYRFLQMSRLPRRLITYFTVERLESGCYICAAFLRAHRIARRQLHDF
LGDSEVARIVIDESNAEGEEARKFLEDVRVTFPQVLRVLKTRQVTYSVLTHLSEYIQNLQKTGLLEEKEMAHLDDAL
QTDLKKFKRNPPLVKMPRVSDLLNTHPLVGALPAAMRDPLLSSTKETVKGHGTILYREGSRPTGIWLVSIGVVKWTS
QRLSSRHSLDPILSHGSTLGLYEVLIGKPYICDMITDSVVHCFFIEAEKIEQLRQSDPSIEIFLWQESALVVARLLL
PMMFEKMATHELRVLITERSTMNIYIKGEEIELEQNFIGILLEGFLKTKNQTLITPPGLLLPPNADLNLFGLESSAI
NRIDYCYTAPSYQVEARARILFVEIGRPEIEADLQRSASLISQTLELPRTQSKEHSGLLSWPESFRKSRGAQNGASL
TEIRDHPASFSARALQLSMYGSMINDMKSGQGQGQRRQRHRHTKASSNKAHSSSYPRVPSRSSNTQRPLLSVQSEGA
NMTTARQAAAAGASLPPEPEEAGRRRRRQRKAIEEDEDNSSDESAGEEVIVRVDSPSMLTFRQPSSAADR
SEQ ID NO: 96-OsSOS1-Q7XBF9
MDNPEAEPDDAVLFVGVSLVLGIASRHLLRGTRVPYTVALLVLGVALGSLEFGTKHGMGKLGAGIRIWANINPDLLL
AVFLPALLFESSFSMEIHQIKKCMAQMVLLAGPGVLISTFFLGSALKLTFPYNWNWKTSLLLGGLLSATDPVAVVAL
LKELGASKKLSTIIEGESLMNDGTAIVVYQLFYRMVLGRTFDAGSIIKFLSEVSLGAVALGLAFGIASVLWLGFIEN
DTIIEIALTLAVSYIAFFTAQDALEVSGVLTVMTLGMFYAAFAKTAFKGDSQQSLHHFWEMVAYIANTLIFILSGVV
IADGVLENNVHFERHGASWGFLLLLYVFVQISRILVVVILYPLLRHFGYGLDLKEATILVWAGLRGAVALSLSLSVK
RASDAVQTHLKPVDGTMFVFFTGGIVFLTLIFNGSTTQFLLHLLGMDRLAATKLRILNYTKYEMLNKALEAFGDLRD
DEELGPPADWVTVKKYITCLNGLDDEPVHPHAVSDRNDRMHTMNLRDIRVRLLNGVQAAYWGMLEEGRITQTTANIL
MRSVDEAMDLVPTQELCDWKGLRSNVHFPNYYRFLQMSRLPRRLITYFTVERLESGCYICAAFLRAHRIARRQLHDF
LGDSEVARIVIDESNAEGEEARKFLEDVRVTFPQVLRVLKTRQVTYSVLTHLSEYIQNLQKTGLLEEKEMAHLDDAL
QTDLKKFKRNPPLVKMPRVSDLLNTHPLVGALPAAMRDPLLSSTKETVKGHGTILYREGSRPTGIWLVSIGVVKWTS
QRLSSRHSLDPILSHGSTLGLYEVLIGKPYICDMITDSVVHCFFIEAEKIEQLRQSDPSIEIFLWQESALVVARLLL
PMMFEKMATHELRVLITERSTMNIYIKGEEIELEQNFIGILLEGFLKTKNQTLITPPGLLLPPNADLNLFGLESSAI
NRIDYCYTAPSYQVEARARILFVEIGRPEIEADLQRSASLISQTLELPRTQSKEHSETIQQLSGAQNGASLTEIRDH
PASFSARALQLSMYGSMINDMKSGQGQGQRRQRHRHTKASSNKAHSSSYPRVPSSSSNTQRPLLSVQSEGANMTTAR
QAAAAGASLPPEPEEAGRRRRRQRKAIEEDEDNSSDESAGEEVIVRVDSPSMLTFRQPSSAADR
SEQ ID NO: 97-OSNHX1-Q9SXJ8
MGMEVAAARLGALYTTSDYASVVSINLFVALLCACIVLGHLLEENRWVNESITALIIGLCTGVVILLMTKGKSSHLF
VFSEDLFFIYLLPPIIFNAGFQVKKKQFFRNFMTITLFGAVGTMISFFTISIAAIAIFSRMNIGTLDVGDFLAIGAI
FSATDSVCTLQVLNQDETPFLYSLVFGEGVVNDATSIVLFNALQNFDLVHIDAAVVLKFLGNFFYLFLSSTFLGVFA
GLLSAYIIKKLYIGRHSTDREVALMMLMAYLSYMLAELLDLSGILTVFFCGIVMSHYTWHNVTESSRVTTKHAFATL
SFIAETFLFLYVGMDALDIEKWEFASDRPGKSIGISSILLGLVLIGRAAFVFPLSFLSNLTKKAPNEKITWRQQVVI
WWAGLMRGAVSIALAYNKFTRSGHTQLHGNAIMITSTITVVLFSTMVFGMMTKPLIRLLLPASGHPVTSEPSSPKSL
HSPLLTSMQGSDLESTTNIVRPSSLRMLLTKPTHTVHYYWRKFDDALMRPMFGGRGFVPFSPGSPTEQSHGGR
SEQ ID NO: 98-OsNHX1-Q6VVA7
MGMEVAAARLGPLYTTSDYASVVSINLFVALLCACIVLGHLLEENRWVNESITALIIGLCTGVVILLMTKGKSSHLF
VFSEDLFFIYLLPPIIFNAGFQVKKKQFFRNEMTITLFGAVGTMISFFTISIAAIAIFSRMNIGTLDVGDFLAIRAI
FPATDSVCTLQVLNQDETPFLYSLVFGEGVVNDATSIVLFNALQNFDLVHIDAAVVLKFLGNFFYLFLSSTFLGVFA
GLLSAYIIKKLYIGRHSTDREVALMMLMAYLSYMLAELLDLSGILTVFFCGIVMSHYTWHNVTESSRVTTKHAFATL
SFIAETFLFLYVGMDALDIEKWEFASDRPGKSIGISSILLGLVLIGRAAFVFPLSFLSNLTKKAPNEKRTWRQQVVI
WWAGLMRGAVSIALAYNKFTRSGHTQLHGNAIMITSTITVVLWSTMVFGMMTKPLIRLLLPASGHPVTSEPSSPKSL
HSPLLTRMQGSDLESTTNIVRPSSLRMLLTKPTHTVHYYWRKFDDALMRPMFGGRGFVPFSPGSPTEQSHGGR
SEQ ID NO: 99-OsHKT1-Q0D9S3
MTSIYHDFIHNKLQSFGRIGRYFVNFVVLAHRFIALHIHPFWIQLSYFLLISILGSVLLMFLKPSNPEFRPGYIDML
FLSTSALTLSSLITIEMEVLSSSQIVVITLLMLLGGEVFVSFLGLMLRLNHKHNPEFSGDKVSSVPIELDTINSAST
VISCEELQLEAAIPEVPSSTIKDLKRSKRLRWFLGFVVFSYFVVIHVAGFLLVLWYISRVSSAKAPLKKKGINIALF
SFSVTVSSFANVGLVPTNENMAIFSKNPGLLLLFIGQILAGNTLYPLFLRLLIWFLGKVTKLRELKLMI
KNPEELQYDYLLPKLPTAFLASTVIGLMASLVTLFGAVDWNSSVEDGLSSYQKIINALFMAVNARHSGENSIDCSLI
APAVLVLFIILMYLPPSTTFALSNGDEKTANKKAKRKLGLVVQNLAFSQLACISVFVIVAFITERSRLRNDPLNFSA
LNMIFEIISAYGNVGLSTGYSCSRLQKLHPGSICQDKPYSLSGWWSDEGKLLLVFVMLYGRLKAFTKGTGEYWRLW
SEQ ID NO: 100-OsSOS2-Q69Q47
MGGEEGMAAGRKKRVGRYEVGRTIGQGTFAKVKFAVDADTGAAVAMKVLDKDTILNHRMLHQIKREISIMKIVRHPN
IVRLNEVLAGKTKIYIILELITGGELFDKIARQGKLRENEARKYFQQLIDAINYCHSKGVYHRDLKPENLLLDSRGN
LKVSDFGLSTLAQKGVGLLHTTCGTPNYVAPEVLSNNGYDGSAADVWSCGVILYVLMAGYLPFEEDDLPTLYDKITA
GQFSCPYWFSPGATSLIHRILDPNPKTRITIEQIREDTWFKKTYVAIKRGEDENVDLDDVQAVEDNIED
KYVSEQVTHNDGGPLVMNAFEMITLSQGLDLSALFDRQQEFVKRQTRFVSRKPAKTIVATIEVVAETMGLKVHSQNY
KLRLEGVSSNRMSPFAVVLQVFEVAPSLFMVDVRKVAGDTLEYHRFYKNLCNKMESIIWRPIEVSAKSALLRTATC
SEQ ID NO: 101-OsAHA3-Q8L613
MAEDKGGLDAVLKESVDLENIPIEEVFQNLKCCRQGLTSEEAQLRLQLFGPNKLEEKEESKFLKFLGFMWNPLSWVM
EAAAIMAIALANGGGKPPDWQDFVGIITLLLINSTISFIEENNAGNAAAALMARLAPKAKVLRNGSWTEEEAAILVP
GDIISIKLGDIIPADARLLEGDPLKIDQSALTGESLPATKGPGDGVYSGSTVKQGEIEAVVIATGVHTFFGKAAHLV
DSTNQVGHFQKVLTAIGNFCICSIAVGMFVEIIVMYPIQHRPYRPGIDNLLVLLIGGIPIAMPTVLSVTMAIGSHRL
SQQGAITKRMTAIEEMAGMDVLCSDKTGTLTLNKLTVDKNLIEIFERGVTQDQVILMAARASRTENQDAIDTAIVGM
LADPKEARAGIQEVHFLPFNPTDKRTALTYIDSDGKMYRVSKGAPEQILNLAHNKTQIERRVHAVIDKFAERGLRSL
AVAYQEVPDGRKESPGGPWRFVALLPLFDPPRHDSAETIRRALNLGVNVKMITGDQLAIGKETGRRLGMGTNMYPSS
ALLGQNKDESVAALPVDDLIEKADGFAGVFPEHKYEIVKRLQARKHICGMTGDGVNDAPALKKADIGIAVADATDAA
RSASDIVLTEPGLSVIISAVLTSRAIFQRMKNYTIYAVSITIRIVFGFMLLALIWEFDFPPFMVLIIAILNDGTIMT
ISKDLVKPSPLPDSWKLAEIFTTGVVLGGYLAMMTVIFFWAAYKTNFFPRIFHVESLEKTAQDDYQKLASAVYLQVS
TISQALIFVTRSRSWSFIERPGFLLVFAFFVAQLIATLIAVYANWAFTSIKGIGWGWAGIVWLYNLVFYFPLDIIKF
LIRYALSGKAWDLVIEQRIAFTRKKDFGKEERELKWAHAHRTLHGLQPPDAKPFPEKTGYSELNQMAEEAKRRAEIA
RLRELHTLKGHVESVVKLKGLDIDTIHQSYTV
SEQ ID NO: 102-OsVHA-A-Q651T8
MSYDRVTTFEDSEKESEYGYVRKVSGPVVVADGMGGAAMYELVRVGNDNLIGEIIRLEGDSATIQVYEETAGLMVND
PVLRTRKPLSVELGPGILGNIFDGIQRPLKTIAIKSGDVYIPRGVSVPALDKDQLWDFEPKKLGVGDAITGGDLYAT
VFENTLMKHHVALPPGSMGKISYIAPAGQYSLQDTVLELEFQGIKKQFTMLQTWPVRSPRPVSSKLAADTPLLTGQR
VLDALFPSVLGGTCAIPGAFGCGKTVISQALSKYSNSEAVVYVGCGERGNEMAEVLMDFPQLTMTLPDGREESVMKR
TTLVANTSNMPVAAREASIYTGITIAEYFRDMGYNVSMMADSTSRWAEALREISGRLAEMPADSGYPAYLAARLASF
YERAGKVKCLGSPDRTGSVTIVGAVSPPGGDFSDPVTSATLSIVQVFWGLDKKLAQRKHFPSVNWLISYSKYSKALE
SFYEKFDQDFIDIRTKAREVLQREDDLNEIVQLVGKDALAESDKITLETAKLLREDYLAQNAFTPYDKFCPFYKSVW
MMRNIIHFNTLANQAVERAANADGQKITYSVIKHRMGDLFYRLVSQKFEDPAEGEDVLVAKFQKLYDDLTTGERNLE
DEAR
SEQ ID NO: 103-OsVHA-B-Q7FV25
MGLVKDGAADLEEGTLEIGMEYRTVSGVAGPLVILDKVKGPKYQEIVNIRLGDGTTRRGQVLEVDGEKAVVQVFEGT
SGIDNKYTTVQFTGEVLKTPVSLDMLGRIFNGSGKPIDNGPPILPEAYLDISGSSINPSERTYPEEMIQTGISTIDV
MNSIARGQKIPLFSAAGLPHNEIAAQICRQAGLVKSLEKGKHAEGGEDDNFAIVFAAMGVNMETAQFFKRDFEENGS
MERVTLFLNLANDPTIERIITPRIALTTAEYLAYECGKHVLVILTDMSSYADALREVSAAREEVPGRRGYPGYMYTD
LATIYERAGRIEGRSGSITQIPILTMPNDDITHPTPDLTGYITEGQIYIDRQLHNRQIYPPINVLPSLSRLMKSAIG
EGMTRRDHSDVSNQLYANYAIGKDVQAMKAVVGEEALSSEDLLYLEFLDKFERKFVTQGAYDTRNIFQSLDLAWTLL
RIFPRELLHRIPAKTLDQYYSRDATH
SEQ ID NO: 104-OsPsbO-A5JV93
MAASLQAAATLMPAKIGGRASSARPSSHVARAFGVDAGARITCSLQSDIREVASKCAEAAKMAGFALATSALLVSGA
SAEGAPKRLTFDEIQSKTYMEVKGTGTANQCPTIDGGVDSFPFKAGKYEMKKFCLEPTSFTVKAEGIQKNEPPAFQK
TKLMTRLTYTLDEMEGPLEVGADGTLKFEEKDGIDYAAVTVQLPGGERVPFLFTVKQLVATGKPESFSGPFLVPSYR
GSSFLDPKGRGGSTGYDNAVALPAGGRGDEEELAKENVKNASSSTGNITLSVTKSKPETGEVIGVFERVQPSDTDLG
AKAPKDVKIQGVWYAQLESN
SEQ ID NO: 105-OsPsbP-XP_002876377.1/Q0KIW5 fragment
VVNASSSEASSDEKNVTRRRLALLGAGALATGLLKSSSAYAEEVPKNYKSYVDSKDGYSYLYPADWRDFDFLGHDSA
FKDRNVALQCVRVGFIPTTKTDIRDLGPMDEAIFNLVNNVYAAPNQIPTVYDMQERTVHGKNYWT
SEQ ID NO: 106-OsPsbQ-P83646
MAQAMASMTGLSQGVQLPAGPRRAGGRSRLAVVRADAAAADVQTGRRAVLGLVATGIAGGALAQAALAEAAKPIKLG
PPPPPSGGLPGTLNSDQARDTDLPLRERFYLQPLPPAEAAARAKESAQDIINLKPLIEKKQWPFVRDDLRLRASYLR
YDLKTVINSKPKDEKKGLKDLTGKLFATIDGLDHAAKIKSPEEAEKYYTLTKSALGDVLAKLG
SEQ ID NO: 107-OsOSK1-Q852Q2
MEGAGRDGNPLGGYRIGKTLGIGSFGKVKIAEHILTGHKVAIKILNRRKIKSMEMEEKVKREIKILRLFMHPHIIRL
YEVIDTPADIYVVMEYVKSGELFDYIVEKGRLQEEEARRFFQQIISGVEYCHRNMVVHRDLKPENLLLDSKCNVKIA
DFGLSNVMRDGHFLKTSCGSPNYAAPEVISGKLYAGPEVDVWSCGVILYALLCGTLPFDDENIPNLFKKIKGGIYTL
PSHLSPLARDLIPRMLVVDPMKRITIREIREHQWFTVGLPRYLAVPPPDTAQQVKKLDDETLNDVINMGFDKNQLIE
SLHKRLQNEATVAYYLLLDNRLRTTSGYLGAEFHESMESSLAQVTPAETPNSATDHRQHGHMESPGFGLRHHFAADR
KWALGLQSRAHPREIITEVLKALQELNVCWKKIGHYNMKCRWSPSFPSHESMMHNNHGFGAESAIIETDDSEKSTHT
VKFEIQLYKTRDEKYLLDLQRVSGPQLLFLDLCSAFLTQLRVL
SEQ ID NO: 108-OsOSK1-Q0DGI1
MEGAGRDGNPLGGYRIGKTLGIGSFGKVKIAEHILTGHKVAIKILNRRKIKSMEMEEKVKREIKILRLFMHPHIIRL
YEVIDTPADIYVVMEYVKSGELFDYIVEKGRLQEEEARRFFQQIISGVEYCHRNMVVHRDLKPENLLLDSKCNVKIA
DFGLSNVMRDGHFLKTSCGSPNYAAPEVISGKLYAGPEVDVWSCGVILYALLCGTLPFDDENIPNLFKKIKGGIYTL
PSHLSPLARDLIPRMLVVDPMKRITIREIREHQWFTVGLPRYLAVPPPDTAQQVKKLDDETLNDVINMGFDKNQLIE
SLHKRLQNEATVAYYLLLDNRLRTTSGYLGAEFHESMESSLAQVTPAETPNSATDHRQHGHMESPGFGLRHHFAADR
KWALGLQSRAHPREIITEVLKALQELNVCWKKIGHYNMKCRWSPSFPSHESMMHNNHGFGAESAIIETDDSEKSTHT
VKFEIQLYKTRDEKYLLDLQRVSGPQLLFLDLCSAFLTQLRVL
SEQ ID NO: 109-OSOSK1-Q9ZTF6
MEGAGSDGNPLGGYRIGKTLGIGSFGKVKIAEHILTGHKVAIKILNRRKSMEMEEKVKREIKILRLFMHPHIIRLYE
VIDTPADIYVVMEYVKSGELFDYDVEKGRLQEEEARRFFQQIISGVEYCHRNMVVRRDLKPENLLLDSKCNVKIADF
GLSNVMRDGHFLKTSCGSPNYAAPEVISGKLYAGPEVDVWSCGVILYALLCGTLPFDDENIPNLFKNIKGGIYTLPS
HLSPLGRDLIPRMLVVDPMKRITIREIREHQWFTVGLPRYLAVPPPDTAQQVKKLDDETQNDVINMGEDKNQLIESL
HKRLQNEATVAYYLLLDNRLRTTSGYLGAEFHESMVSSLAQVTPAETPNSATDHRQHGHMESPGFGLRHHFAADRKW
ALGLQSRAHPREIITEVLKALQELNVCWKKIGHHNMKCRWSPSFPSHESMMHNNHGFGAASAMIETDDSEKSTHTVK
FEIQLYKTRDEKYFLDLQRLSGPQLLFLDLCSAFLTQLRVL
SEQ ID NO: 110-OsOSK1-Q9ZRJ1
MEGAGRDGNPLGGYRIGKTLGIGSFGKVKIAEHILTGHKVAIKILNRRKIKSMEMEEKVKREIKILRLFMHPHIIRL
YEVIDTPADIYVVMEYVKSGELFDYIVEKGRLQEEEARRFFQQIISGVEYCHRNMVVHRDLKPENLLLDSKCNVKIA
DFGLSNVMRDGHFLKTSCGSPNYAAPEVISGKLYAGPEVDVWSCGVILYALLCGTLPFDDENIPNLFKKIKGGIYTL
PSHLSPLARDLIPRMLVVDPMKRITIREIREHQWFTVGLPRYLAVPPPDTAQQVKKLDDETLNDVINMGFDKNQLIE
SLHKRLQNEATVAYYLLLDNRLRTTSGYLGAEFHESMESSLAQVTPAETPNSATDHRQHGHMESPGFGLRHHFAADR
KWALGLQSRAHPREIITEVLKALQELNVCWKKIGHYNMKCRWSPSFPSHESMMHNNHGFGAESAIIETDDSEKSTHT
VKFEIQLYKTRDEKYLLDLQRVSGPQLLFLDLCSAFLTQLRVL
SEQ ID NO: 112-P450-Q9FVS9
MAMLGFYVTFIFFLVCLFTYFFLQKKPQGQPILKNWPFLRMLPGMLHQIPRIYDWTVEVLEATNLTFYFKGPWLSGT
DMLFTADPRNIHHILSSNFGNYPKGPEFKKIFDVLGEGILTVDFELWEEMRKSNHALFHNQDFIELSVSSNKSKLKE
GLVPFLDNAAQKNIIIELQDVFQRFMEDTSSILMTGYDPMSLSIEMLEVEFGEAADIGEEAIYYRHFKPVILWRLQN
WIGIGLERKMRTALATVNRMFAKIISSRRKEEISRAKTEPYSKDALTYYMNVDTSKYKLLKPNKDKFIRDVIFSLVL
AGRDTTSSVLTWFFWLLSKHPQVMAKLRHEINTKFDNEDLEKLVYLHAALSESMRLYPPLPFNHKSPAKPDVLPSGH
KVDANSKIVICIYALGRMRSVWGEDALDFKPERWISDNGGLRHEPSYKFMAFNSGPRTCLGKNLALLQMKMVALEII
RNYDFKVIEGHKVEPIPSILLRMKHGLKVTVTKKI
SEQ ID NO: 113-Oryza sativa sucrose-phosphate synthase 1-QJGK4
MAGNEWINGYLEAILDSGGAAGGGGGGGGGGGGGGGGGGGGGGGGVDPRSPAAGAASPRGPHMNENPTHYFVEEVVK
GVDESDLHRTWIKVVATRNARERSTRLENMCWRIWHLARKKKQLELEGILRISARRKEQEQVRRETSEDLAEDLFEG
EKADTVGELAQQDTPMKKKFQRNFSELTVSWSDENKEKKLYIVLISLHGLVRGDNMELGRDSDTGGQVKYVVELARA
LAMMPGVYRVDLFTRQVSSPEVDWSYGEPTEMLTSGSTDGEGSGESAGAYIVRIPCGPRDKYLRKEALWPYLQEFVD
GALAHILNMSKALGEQVSNGKLVLPYVIHGHYADAGDVAALLSGALNVPMVLTGHSLGRNKLEQIMKQGRMSKEEID
STYKIMRRIEGEELALDAAELVITSTRQEIDEQWGLYDGFDVKLEKVLRARARRGVSCHGRFMPRMVVIPPGMDESS
VVVPEDTSDGDDGKDFEIASPRSLPPIWAEVMRFLTNPHKPMILALSRPDPKKNITTLVKAFGECRPLRELANLILI
MGNRDDIDEMSAGNASVLTTVLKLIDKYDLYGSVAFPKHHKQSDVPEIYRLTGKMKGVFINPALVEPFGLTLIEAAA
HGLPIVATKNGGPVDIKNALNNGLLVDPHDQHAIADALLKLVADKNLWQECRKNGLRNIQLYSWPEHCRTYLTRIAG
CRIRNPRWLMDTPADAAAEEEEALEDSLMDVQDLSLRLSIDGERGSSMNDAPSSDPQDSVQRIMNKIKRSSPADTDG
AKIPAEAAATATSGAMNKYPLLRRRRRLFVIAVDCYGDDGSASKRMLQVIQEVFRAVRSDSQMSRISGFALSTAMPL
PETLKLLQLGKIPPTDFDALICGSGSEVYYPSTAQCVDAGGRLRPDQDYLLHINHRWSHDGAKQTIAKLAHDGSGTN
VEPDVESCNPHCVSFFIKDPNKVRTIDEMRERVRMRGLRCHLMYCRNATRLQVVPLLASRSQALRYLFVRWGLSVGN
MYLIVGEHGDTDHEEMLSGLHKTVIIRGVTEKGSEQLVRSSGSYQREDVVPSESPLIAFTKGDLKADEIMRALKEVT
KAASGM
SEQ ID NO: 114-Oryza sativa delta-1-pyrroline-5-carboxylate synthase 1 O04226
MASVDPSRSFVRDVKRVIIKVGTAVVSRQDGRLALGRVGALCEQVKELNSLGYEVILVTSGAVGVGRQRLRYRKLVN
SSFADLQKPQMELDGKACAAVGQSGLMALYDMLFNQLDVSSSQLLVTDSDFENPKFREQLTETVESLLDLKVIPIEN
ENDAISTRKAPYEDSSGIFWDNDSLAGLLALELKADLLILLSDVDGLYSGPPSEPSSKIIHTYIKEKHQQEITFGDK
SRVGRGGMTAKVKAAVLASNSGTPVVITSGFENRSILKVLHGEKIGTLFHKNANLWESSKDVSTREMAVAARDCSRH
LQNLSSEERKKILLDVADALEANEDLIRSENEADVAAAQVAGYEKPLVARLTIKPGKIASLAKSIRTLANMEDPINQ
ILKKTEVADDLVLEKTSCPLGVLLIVFESRPDALVQIASLAIRSGNGLLLKGGKEAIRSNTILHKVITDAIPRNVGE
KLIGLVTTRDEIADLLKLDDVIDLVIPRGSNKLVSQIKASTKIPVLGHADGICHVYIDKSADMDMAKHIVMDAKIDY
PAACNAMETLLVHKDLMKSPGLDDILVALKTEGVNIYGGPIAHKALGEPKAVSFHHEYSSMACTVEFVDDVQSAIDH
IHRYGSAHTDCIVTTDDKVAETFLRRVDSAAVFHNASTRESDGARFGLGAEVGISTGRIHARGPVGVEGLLTTRWIL
RGRGQVVNGDKDVVYTHKSLPLQ

While the invention has been described with respect to growing plants in the sea, the invention may also be used to grow plants in freshwater or brackish water. In particular, large freshwater or brackish bodies with significant wave action may still pose may of the same challenges as the sea, which can be overcome through the use of the present invention as described above. The salinity of such a body of water may be The salinity of the body of water may be less than or equal to 0.1, less than or equal to 0.5, less than or equal to 2, less than or equal to 5, less than or equal to 8, less than or equal to 12, less than or equal to 18, less than or equal to 25, less than or equal to 30, or less than or equal to 35 parts per thousand. References to the sea in the above description and the following claims may therefore be replaced by reference to freshwater or brackish water if necessary. Similarly, references to the sea in the above description or following claims may be replaced by reference to the oceans, a river, a mouth, delta or estuary of a river (such as a saltwater or brackish mouth, delta or estuary of a river), a coastline or a strait of an ocean or sea.

While the preceding description contains many specifics, these specifics should not be construed as limitations on the scope of the invention, but merely as examples of embodiments of the invention. Those skilled in the art will envision other possible variations and equivalents that are within the scope and spirit of the invention as defined by the following claims.

Glossary of Reference Numerals used in FIGS.

| | |
|---|---|
| 10 | Pod |
| 20 | Mesh |
| 22 | Upper Surface |
| 24 | Lower Surface |
| 26 | Side Surface |
| 30 | Upper Portion |
| 40 | Lower Portion |
| 41 | Hole |
| LA-LA | Longitudinal Axis of Pod |
| 100 | Support Structure |
| 110 | Pod Connection Portion |
| 120 | Structure Connection Portion |
| 122 | Flange |
| 124 | Hole |
| 130 | Corner |
| 140 | Top Surface |
| 150 | Pod Connection hole |
| 152 | Ledge |
| 160 | Hole |
| 200 | Plurality of Structural Components |
| 210 | Centre Beam |
| 212 | Brace Slots |
| 214 | Slot Entrance |
| 216 | Slot End |
| 218 | Tray Slot |
| LB-LB | Longitudinal Axis of Centre Beam |
| 220 | Side Segment |
| 222 | Brace Slot |
| 224 | Slot Entrance |
| 226 | Slot End |
| 228 | Tray Slot |
| LC-LC | Longitudinal Axis of Side Portion |
| 230 | Brace Segment |
| 232 | Protrusion |
| 240 | Tray |
| 242 | Tray Male Connection |
| 244 | Tray Female Connection |
| 1000 | Assembly |
| 1002 | Pin |
| 1004 | Central Vertex |

Aspects

The following list of aspects forms part of the description:

Pod

1. A plant propagation pod for germinating a seed and structurally supporting a subsequent plant, the pod comprising:
a three-dimensional mesh configured to structurally support the roots of a plant;
wherein the mesh comprises an upper surface, a lower surface and at least one side surface.

2. The pod of aspect 1, wherein the upper and lower surfaces are planar.

3. The pod of aspect 2, wherein the upper and lower surfaces are parallel to each other.

4. The pod of any preceding aspect, wherein the pod comprises a plurality of outer surfaces.

5. The pod of aspect 4, wherein the upper and lower surfaces each form one of the plurality of outer surfaces of the pod.

6. The pod of aspect 4 or 5, wherein the at least one side surface forms one of the plurality of outer surfaces of the pod.

7. The pod of any preceding aspect, wherein the pod comprises an upper portion.

8. The pod of aspect or 7, wherein the at least one side surface forms at least one side wall of the upper portion.

9. The pod of aspect 7 or 8, wherein the upper portion is prismatic.

10. The pod of aspect 9, wherein the prismatic portion is a cylindrical portion, and wherein the at least one side surface defines the side wall of the cylindrical portion.

11. The pod of any of aspects 7 to 10, wherein the pod further comprises a lower portion connected to the upper portion.

12. The pod of aspect 11, wherein the pod comprises a longitudinal axis extending through the upper and lower surfaces, and wherein the lower portion of the mesh tapers towards the longitudinal axis of the pod as the mesh extends away from the upper portion.

13. The pod of aspect 11 or 12, wherein the lower portion of the mesh has the shape of a truncated cone or truncated pyramid.

14. The pod of any preceding aspect, wherein the mesh has a total mesh volume and comprises mesh material and void space.

15. The pod of aspect 14, wherein the volume fraction of mesh material to total mesh volume is between 10% and 50%, optionally 20% and 40%, further optionally between 25 and 35%.

16. The pod of aspect 14 or 15, wherein the total mesh volume occupies at least 10, 20, 30, 40, 50, 60, 70, 80, 90% of a volume of the pod.

17. The pod of any preceding aspect, wherein the pod consists of the mesh.

18. The pod of any preceding aspect, wherein the mesh has a lattice cell minimum dimension or maximum dimension of 1 mm or more to 5 mm or less, optionally 2 mm or more to 4 mm or less, further optionally 2.5 mm or more to 3.5 mm or less.

19. The pod of any preceding aspect, wherein the longitudinal axis extends through at least 2, at least 3, at least 4, at least 5, at least 6, at least 7, at least 8, at least 9, at least 10, at least 15, at least 20, at least 25, at least 30, at least 40 or at least 50 lattice cells as it extends between the upper and lower surface

20. The pod of any preceding aspect, wherein the mesh is configured to structurally support the plant through the roots of the plant embedding in the mesh.

21. The pod of any preceding aspect, further comprising a hole in the upper surface of the mesh for germinating a seed.

22. The pod of aspect 21, wherein the hole is positioned in the centre of the upper surface of the mesh.

23. The pod of aspect 21 or 22, wherein the hole extends a plurality of lattice cells into the mesh.

24. The pod of any of aspects 21 to 23, wherein the hole extends into mesh 20 no more than 5%, no more than 10%, no more than 15%, no more than 20%, no more than 25% or no more than 30% of the distance from upper surface 22 to lower surface 24.

25. The pod of any preceding aspect, wherein the pod further comprises a wall with an outer surface and an inner surface, the mesh being connected to the inner surface of the wall.

26. The pod of aspect 25, wherein the mesh extends from the inner surface of the wall, optionally wherein the mesh is in physical contact with and extends from the inner surface of the wall around the full perimeter of the inner surface.

27. The pod of aspect 25 or 26, wherein the mesh is integrally formed with the inner surface of the wall.

28. The pod of any preceding aspect, wherein the mesh comprises a tetrahedral lattice, a gyroid lattice, a square lattice, an octet lattice, an isotruss lattice, a Voronoi lattice, a fluorite lattice, a Delaunay lattice, or a simple cubic lattice.

29. The pod of any preceding aspect, wherein the pod is inflexible.

30. The pod of any preceding aspect, wherein the pod resiliently deforms under loading.

31. The pod of any preceding aspect, wherein the pod comprises a synthetic material.

32. The pod of any preceding aspect, wherein the pod comprises a plastic, optionally HDPE, ABS, PLA, biodegradable plastic, and/or plant derived plastic.

33. The pod of any preceding aspect, wherein the mesh comprises or consists of a synthetic material.

34. The pod of any preceding aspect, wherein the mesh comprises plastic, optionally HDPE, ABS, PLA, biodegradable plastic, and/or plant derived plastic.

35. The pod of any preceding aspect, wherein the pod or mesh comprises a rigid plastic, optionally HDPE, ABS, biodegradable plastic, and/or plant derived plastic.

36. The pod of any preceding aspect, wherein the pod is configured to connect with a floating support structure that allows the plant to be cultivated on water.

37. The pod of any preceding aspect, wherein the pod comprises connection means to allow the pod to connect to a floating support structure that allows the plant to be cultivated on water.

38. The pod of aspect 37, wherein the connection means comprises a screw thread, a ridge or a protrusion.

39. The pod of any preceding aspect, wherein the distance between the upper and lower surface defines a height of the mesh, and wherein the mesh further comprises a width and length perpendicular to each other and perpendicular to the height.

40. The pod of any preceding aspect, wherein the width or length of the pod divided by the height of the pod is 0.1 or more to 10 or less, optionally 0.2 or more to 5 or less, optionally 0.25 or more to 4 or less, further optionally 0.33 or more to 3 or less, further optionally 0.5 or more to 2 or less, further optionally 0.66 or more to 1.5 or less.

41. The pod of aspect 39 or 40, wherein the height of the pod is 2 cm or more and 20 cm or less, optionally 2 cm or more and 5 cm or less, optionally 2 cm or more and 10 cm or less, optionally 3 cm or more and 8 cm or less, further optionally 4 cm or more and 6 cm or less.

42. The pod of any of aspects 39 to 41, wherein the width or length of the pod is 2 cm or more and 20 cm or less, optionally 2.5 cm or more and 10 cm or less, optionally 3 cm or more and 8 cm or less, further optionally 4 cm or more and 6 cm or less.

43. The pod of any preceding aspect, wherein the plant is a rice plant.

44. The pod of any preceding aspect, wherein the plant is a single plant, or wherein the plant comprises or consists of 2, 3, 4, 5, 6, 7, 8, 9 or 10 plants.

45. The pod of any preceding aspect, wherein the plant is a genetically engineered salt tolerant plant.

46. The pod of aspect 45, wherein the genetically engineered salt tolerant plant is a genetically engineered plant comprising at least three genes of interest, wherein each gene of interest is operatively linked to an enhancer element, wherein the engineered plant has increased salt tolerance compared to a plant of a same species without said genome modifications.

47. The pod of aspect 46, wherein the at least three genes of interest are selected from the group consisting of:
(a) a gene that encodes a plasma membrane ion transporter;
(b) a gene that encodes a plasma membrane hydrogen exporting ATPase;
(c) a gene that encodes a protein kinase;
(d) a gene that encodes a vacuolar hydrogen exporting ATPase;
(e) a gene that encodes a vacuolar sodium/proton transporter;
(f) a gene that encodes a potassium transporter; and
(g) a gene that encodes an antioxidant.

48. The pod of any preceding aspect, further comprising the plant, the roots of the plant being embedded in the mesh such that the pod can structurally support the plant.

49. The pod of any preceding aspect, wherein at least some, optionally all, of the pod is injection molded.

Support Structure

50. A support structure for growing a plant in the sea, comprising:
at least one pod connection portion configured to connect to a plant propagation pod according to any preceding aspect;
wherein the support structure is configured to float and protrude from the surface of water.

51. A support structure for growing a plant in the sea, comprising:
at least one pod connection portion configured to connect to a plant propagation pod;
wherein the support structure is configured to float and protrude from the surface of water.

52. The support structure of aspect 50 or 51, further comprising at least one plant propagation pod according to any of aspects 1 to 49 connected to the at least one pod connection portion, or a plant propagation pod according to any of aspects 1 to 49 connected to each pod connection portion.

53. The support structure of any of aspects 50 to 52, wherein the at least one pod connection portion is configured to removably connect to the at least one pod.

54. The support structure of aspect 52, wherein the at least one pod is fixedly connected to the at least one pod connection portion, optionally wherein the plant propagation pod is integrally formed with the support structure, further optionally wherein the mesh is an integral part of the support structure.

55. The support structure of any of aspects 52 to 54, where in the support structure comprises or consists of plastic, optionally HDPE.

56. The support structure of any of aspects 52 to 55, wherein at least part of the support structure is hollow.

57. The support structure of any of aspects 52 to 56, wherein the support structure comprises at least one support structure connection portion configured to enable connection of the support structure to a second support structure.

58. The support structure of aspect 57, wherein the support structure comprises a plurality of corners, and wherein the support structure comprises a support structure connection portion at each of the corners.

59. The support structure of aspect 57, wherein the support structure comprises a plurality of corners forming multiple pairs of corners around the perimeter of the support structure, and wherein the support structure comprises a support structure connection portion located between each pair of corners.

60. The support structure of aspect 57 or 59, wherein the at least one support structure connection portion comprises a hole in the support structure, optionally wherein the support structure connection portion comprises a flange, with the hole being in the flange.

61. The support structure of any of aspects 52 to 60, wherein the at least one pod connection portion comprises 2 or more, 3 or more, 4 or more 5 or more, 6 or more, 7 or more, 8 or more, 9 or more, 10 or more, 15 or more, 20 or more, 25 or more or 30 or more pod connection portions.

62. The support structure of aspect 61 when dependent on aspect 52, wherein the at least one pod comprises a plurality of pods, with a respective pod of the plurality of pods connected to each respective pod connection portion, optionally wherein each pod is removably connected to the at least one pod connection portion.

63. The support structure according to any of aspects 50 to 62, wherein the support structure comprises a top surface.

64. The support structure according to aspect 63, wherein when the support structure is floating on a stationary fluid, the top surface is parallel to the surface of the fluid.

65. The support structure according to aspect 63 or 64, wherein the top surface comprises the at least one pod connection portion.

66. The support structure of any of aspects 63 to 65, wherein the at least one pod connection portion is at least one pod receiving hole extending through the top surface, wherein the pod receiving hole is a through hole.

67. The support structure of aspect 66, wherein the perimeter at the top surface of the at least one pod receiving hole matches the perimeter of the at least one pod.

68. The support structure of aspect 66 or aspect 67, wherein the at least one pod receiving hole comprises a ledge, wherein the ledge is configured to support the at least one pod when received by the at least one pod receiving hole.

69. The support structure of any of aspects 66 to 68, wherein the upper surface is level with the top surface of the support structure when the at least one pod is received by the at least one pod receiving hole.

70. The support structure of any of aspects 66 to 69, wherein the mesh protrudes beyond the pod receiving hole in the direction away from the top surface when the at least one pod is received by the at least one pod receiving hole.

71. The support structure according to any of aspects 50 to 70, wherein the support structure further comprises one or more holes that pass fully through the support structure, wherein the combined area of the one or more holes is at least 5, 10, 15, 20, 25, 30, 35, 40, 45, 50, 55 or 60% of the area enclosed by the perimeter of the support structure.

72. The support structure according to aspect 71, wherein the one or more holes pass through the top surface of the support structure.

73. The support structure of any of aspects 50 to 72, wherein the support structure comprises a plurality of structural components that allow the support structure to be securely assembled without fasteners.

74. The support structure of aspect 73, wherein one or more, and optionally all, of the structural components are hollow.

75. The support structure of any of aspects 50 to 74, wherein the plurality of structural components can be slotted together to securely assemble the support structure.

76. The support structure of aspect 73 or aspect 75, wherein the plurality of structural components comprises at least two side segments and at least two brace segments, each of the side segments and brace segments defining a longitudinal axis, wherein each side segment is configured to slidably connect with an interference fit to two of the at least two brace segments using respective slots that are perpendicular to both the longitudinal axis of the respective side segment and the longitudinal axis of the respective brace segment.

77. The support structure of aspect 76, wherein the plurality of structural components further comprises at least one tray, wherein the at least one tray comprises the at least one pod connection portion.

78. The support structure of aspect 77, wherein the at least one tray is configured to slidably connect to at least one side segment using a tray slot that is parallel to the longitudinal axis of side segment.

79. The support structure of aspect 78, wherein the at least two side segments and at least two brace segments respectively consist of two side segments and two brace segments, wherein the at least one tray consists of one tray, wherein the at least one tray is configured to slidably connect to both side segments using respective tray slots that are parallel to the longitudinal axis of side segment.

80. The support structure of aspect 78, wherein the plurality of structural components further comprises a centre beam defining a longitudinal axis, wherein the at least two brace segments comprise four brace segments, wherein the at least one tray comprises two trays, wherein the at least one pod connection portion is at least two pod connection portions and each of the two trays comprise at least one pod connection portion, wherein each brace segment slidably connects with an interference fit to the centre beam using a respective slot that is perpendicular to the longitudinal axis of the centre beam, and wherein each tray is configured to slidably connect to the centre beam using a respective tray slot that is parallel to the longitudinal axis of centre beam.

81. The support structure of aspect 80, wherein the structural components consist of two side segments, four brace segments, the centre beam and the two trays.

82. The support structure of any of aspects 77 to 81 when dependent on aspect 57, wherein the at least one tray comprises two connection portions, each tray connection portion configured to connect to a tray of an adjacent support structure if the support structure is connected to the adjacent support structure via a support structure connection portion.

83. The support structure of aspect 82, wherein the at least two tray connection portions comprise a male tray connection portion and a female tray connection portion

84. The support structure of any of aspects 50 to 83, wherein the support structure has a shape that is a square, a triangle, an equilateral triangle, a hexagon, a regular hexagon or a rectangle.

85. The support structure of any of aspects 50 to 84, wherein the support structure has a maximum dimension parallel to the top surface of 10 cm or more and 150 cm or less, optionally 20 cm or more and 100 cm or less, optionally 25 cm or more and 50 cm or less, optionally 30 cm or more and 40 cm or less.

86. The support structure of any of aspect 50 to 85 when dependent on aspect 61, wherein a first of the two or more pod connection portions is a minimum distance from a second of the two or mod pod connection portions, and wherein the minimum distance is 0 cm or more and 30 cm or less, optionally 5 cm or more and 25 cm or less, optionally 10 cm or more and 20 cm or less, optionally 12.5 cm or more and 17.5 cm.

87. The support structure of aspect 86, wherein each of the two or more pod connection portions are arranged so that each pod connection portion is no closer than the minimum distance to any of the other pod connection portions.

88. A kit of parts for the support structure of any of aspects 50 to 87.

89. A method of constructing the support structure of any of aspects 73 to 83, comprising assembling the structural components without the use of fasteners to construct the support structure.

Assembly

90. An assembly for growing crops in the sea, comprising:
a plurality of support structures, each support structure being a support structure according to any of aspects 50 to 87;
wherein each support structure is connected to and in direct contact with at least one other support structure of the plurality of support structures.

91. The assembly of aspect 90 when dependent on aspect 57, wherein the connection portion of each support structure allows the support structure to flex relative to a neighbouring support structure as a result of wave action.

92. The assembly of aspect 90 or 91, wherein the plurality of support structures comprises three support structures, wherein each of the three support structures are connected to and in direct contact with the other two of the three support structures.

93. The assembly of aspect 92, wherein each of the three support structures tessellate together.

94. The assembly of aspect 93, wherein each of the three support structures have the same shape.

95. The assembly of aspect 92 or 93, when dependent on aspect 57, wherein the three support structures comprise a central vertex, wherein each support structure comprises a connection portion of the at least one support structure connection portion at the central vertex, and wherein the connection portions at the central vertex are joined together.

96. The assembly of any of aspect 90 to 95 when dependent on aspect 57, wherein each support structure comprises at least two vertices in contact with each support structure in which it is in contact, wherein each support structure comprises a connection portion at each of the at least two vertices, and wherein the connection portions at the respective at least two vertices are joined together.

97. The assembly of any of aspect 90 to 96 when dependent on claim 57, wherein the support structure connection portions are permanently or removably connected together, optionally by a pin and cap, optionally wherein the pin comprises a screw and the cap comprises a nut.

98. The assembly of any of aspects 92 to 97, wherein each of the three support structures is configured to simultaneously flex relative to relative to the other two of the three support structures.

99. The assembly of aspect 98, wherein each of the three support structures is capable of flexing at an angle of 5 degrees or more and 20 degrees or less relative to the at least two other support structures, optionally an angle of 7.5 degrees or more and 10 degrees or less.

100. The assembly of any of aspects 92 to 99 when dependent on aspect 82, wherein a tray of the at least one tray of a first support structures of the plurality of support structures is connected to a tray of the at least one tray of a second support structure of the plurality of support structures, the first and second support structure being connected to and in direct contact with each other.

101. The assembly of aspect 100, wherein the tray slots of the first support structure and the tray slots of the second support structure are parallel with each other.

102. A kit of part for the assembly of any of aspects 90 to 101.

103. A method of constructing the assembly of any of aspects 90 to 101, comprising connecting the plurality of support structures together such that each support structure is connected to and in direct contact with at least one other support structure of the plurality of support structures.

Method

104. A method, comprising:
floating a support structure according to any of aspects 50 to 85 or an assembly of support structures according to any of aspects 90 to 101 on a body of water, wherein the support structure protrudes from the surface of the water;

105. The method according to aspect 104, wherein the body of water is salt water, optionally the sea or the ocean.

106. The method of aspect 104, wherein the salinity of the body of water is greater than 2, 5, 8, 12, 18, 25 30 or 35 parts per thousand.

107. The method of any of aspects 104 to 106, further comprising growing at least one plant in the at least one pod.

108. The method according to aspects 104 to 107 when dependent on aspect 52 or aspect 54, wherein the method further comprises germinating at least one plant in the at least one pod.

109. The method of aspect 108, wherein germinating the at least one plant in the at least one pod comprises germinating the plant in the at least pod on land.

110. The method of aspect 108 or 109, wherein the method further comprises connecting the at least one pod to the at least one pod connection portion after germination has occurred, optionally after the floating of the support structure or assembly.

111. The method of any of aspects 107 to 110, wherein the method further comprising harvesting the plant.

112. The method of aspect 111, wherein harvesting the plant comprises removing the at least one plant from the support structure while the support structure continues to float on the body of water.

113. The method of aspect 111, wherein harvesting the plant comprises removing the support structure or assembly from the body of water, and removing the at least one plant from the support structure or assembly while the support structure is not in the body of water.

114. The method of aspect 112 or 113, wherein removing the at least one plant from the support structure comprises removing the at least one pod from the at least one pod connection portion.

115. The method of any of aspects 104 to 114, wherein the plant is a rice plant.

116. The method of any of aspects 107 to 115, wherein the at least one plant grown in the at least one pod is a single plant.

117. The method of any of aspects 104 to aspect 116, wherein the plant is a genetically engineered salt tolerant plant.

118. The method of aspect 117, wherein the genetically engineered salt tolerant plant is a genetically engineered plant comprising at least three genes of interest, wherein each gene of interest is operatively linked to an enhancer element, wherein the engineered plant has increased salt tolerance compared to a plant of a same species without said genome modifications.

119. The method of aspect 118, wherein the at least three genes of interest are selected from the group consisting of:
(a) a gene that encodes a plasma membrane ion transporter;
(b) a gene that encodes a plasma membrane hydrogen exporting ATPase;
(c) a gene that encodes a protein kinase;
(d) a gene that encodes a vacuolar hydrogen exporting ATPase;
(e) a gene that encodes a vacuolar sodium/proton transporter;
(f) a gene that encodes a potassium transporter; and
(g) a gene that encodes an antioxidant.

What is claimed is:

1. A plant propagation pod for germinating a seed and structurally supporting a subsequent plant, the pod comprising:
a three-dimensional mesh configured to structurally support the roots of a plant;
wherein the mesh comprises an upper surface, a lower surface and at least one side surface.

2. The pod of claim 1, wherein the pod comprises an upper portion, wherein the upper portion is prismatic.

3. The pod of claim 2, wherein the pod further comprises a lower portion connected to the upper portion, wherein the pod comprises a longitudinal axis extending through the upper and lower surfaces, and wherein the lower portion of the mesh tapers towards the longitudinal axis of the pod as the mesh extends away from the upper portion.

4. The pod of claim 1, wherein the mesh has a total mesh volume and comprises mesh material and void space, wherein the volume fraction of mesh material to total mesh volume is between 10% and 50%.

5. The pod of claim 1, wherein the mesh comprises lattice cells, and wherein a longitudinal axis of the pod extends through at least 5 lattice cells as it extends between the upper and lower surface

6. The pod of claim 1, wherein the mesh is configured to structurally support the plant through the roots of the plant embedding in the mesh.

7. The pod of claim 1, further comprising a hole in the upper surface of the mesh for germinating a seed, wherein the hole extends a plurality of lattice cells into the mesh.

8. The pod of claim 1, wherein the pod resiliently deforms under loading.

9. The pod of claim 1, wherein the mesh comprises a synthetic material.

10. The pod of claim 1, wherein the plant is a rice plant.

11. The pod of claim 1, wherein the plant is a genetically engineered salt tolerant plant.

12. A support structure for growing a plant in the sea, comprising:
at least one pod connection portion;
at least one plant propagation pod according to claim 1 connected to the at least one pod connection portion;
wherein the support structure is configured to float and protrude from the surface of water.

13. The support structure of claim 12, wherein the at least one pod is removably connected to the at least one pod connection portion.

14. The support structure of claim 12, wherein at least part of the support structure is hollow.

15. The support structure of claim 12, wherein the at least one pod connection portion comprises 2 or more pod connection portions, wherein the at least one pod comprises a plurality of pods, with a respective pod of the plurality of pods connected to each respective pod connection portion.

16. The support structure according to claim 12, wherein the support structure comprises a top surface, wherein the top surface comprises the at least one pod connection portion, and wherein each pod connection portion is a pod receiving hole extending through the top surface, wherein the pod receiving hole is a through hole.

17. The support structure of claim 16, wherein the mesh protrudes beyond the pod receiving hole in the direction away from the top surface.

18. An assembly for growing crops in the sea, comprising:
a plurality of support structures, each support structure being a support structure according to claim 12;
wherein each support structure of the plurality of support structures comprises at least one support structure connection portion configured to enable connection of the support structure to a second support structure, wherein the connection portion of each support structure allows the support structure to flex relative to a neighbouring support structure as a result of wave action, wherein each support structure is connected to and in direct contact with at least one other support structure of the plurality of support structures through the at least one support structure connection portion;
wherein the plurality of support structures comprises three support structures, wherein each of the three support structures are connected to and in direct contact with the other two of the three support structures, wherein each of the three support structures tessellate together.

19. The assembly of claim 18, wherein the support structure connection portions are removably connected together.

20. A method, comprising:
floating a support structure according to claim 12 on a body of water, wherein the support structure protrudes from the surface of the water, wherein the body of water is the sea;
growing at least one plant in the at least one pod.

21. The method according to claim 20, wherein the method further comprises germinating at least one plant in the at least one pod, wherein germinating the at least one plant in the at least one pod comprises germinating the plant in the at least pod on land, wherein the method further comprises connecting the at least one pod to the at least one pod connection portion after germination has occurred, and after the floating of the support structure.

22. The method of claim 20, wherein the method further comprising harvesting the plant, wherein harvesting the plant comprises removing the at least one plant from the support structure, wherein removing the at least one plant from the support structure comprises removing the at least one pod from the at least one pod connection portion.
